SEQUENCE LISTING (5 sequences)

SEQ ID NO: 1 - protein, 84 aa, Artificial Sequence
  Description of Artificial Sequence Synthetic polypeptide
NVKRRTHNVLERQRRNELKRSFFALRDQIPELENNEKAPKVVILKKATAYILSVQAEEQKLISEEDLLRKRREQLKHKLEQLGG

SEQ ID NO: 2 - protein, 82 aa, Artificial Sequence
  Description of Artificial Sequence Synthetic polypeptide
DKRAHHNALERKRRDHIKDSFHSLRDSVPSLQGEKASRAQILDKATEYIQYMRRKNHTHQQDIDDLKRQNALLEQQVRALGG

SEQ ID NO: 3 - protein, 90 aa, Artificial Sequence
  Description of Artificial Sequence Synthetic polypeptide
MATEENVKRRTHNVLERQRRNELKRSFFALRDQIPELENNEKAPKVVILKKATAYILSVQAETQKLISEIDLLRKQNEQLKHKLEQLRNS

SEQ ID NO: 4 - protein, 40 aa, Artificial Sequence
  Description of Artificial Sequence Synthetic polypeptide
  MOD_RES (33)..(33): 6-aminohexanoic acid
  MOD_RES (38)..(38): 6-aminohexanoic acid
QKKRKSKANKKNWPKGKLSIHAKDYKQGPKAKXRKQRXRG

SEQ ID NO: 5 - DNA, 22 bp, Artificial Sequence
  Description of Artificial Sequence Synthetic probe
ccggctgacacgtggtattaat

(19) United States
(12) Patent Application Publication
(10) Pub. No.: US 2024/0336649 A1
(43) Pub. Date: Oct. 10, 2024

(54) SYNTHESIS OF COVALENT PROTEIN DIMERS THAT CAN INHIBIT MYC-DRIVEN TRANSCRIPTION

(71) Applicants: MASSACHUSETTS INSTITUTE OF TECHNOLOGY, CAMBRIDGE, MA (US); WHITEHEAD INSTITUTE OF BIOMEDICAL RESEARCH, CAMBRIDGE, MA (US)

(72) Inventors: ANDREI LOAS, CAMBRIDGE, MA (US); BRADLEY L. PENTELUTE, REVERE, MA (US); SEBASTIAN POMPLUN, CAMBRIDGE, MA (US); MUHAMMAD JBARA, CAMBRIDGE, MA (US); CARLY KATHERINE SCHISSEL, CAMBRIDGE, MA (US); JACOB JOSHUA LEE RODRIQUEZ, SOMERVILLE, MA (US); STEPHEN LEFFLER BUCHWALD, NEWTON, MA (US); ANN BOIJA, CAMBRIDGE, MA (US); ISAAC KLEIN, CAMBRIDGE, MA (US); SUSANA WILSON HAWKEN, CAMBRIDGE, MA (US); CHARLES HAN LI, CAMBRIDGE, MA (US)

(21) Appl. No.: 18/572,247

(22) PCT Filed: Jun. 17, 2022
(86) PCT No.: PCT/US2022/033920

§ 371 Date: Dec. 20, 2023

Related U.S. Application Data
(60) Provisional application No. 63/213,024, filed on Jun. 21, 2021.

(51) Int. Cl. C07K 1/10; A61K 38/00; A61K 49/00; A61P 35/00; C07K 1/06; C07K 1/107; C07K 14/00

(52) U.S. Cl. CPC C07K 1/10; A61K 49/0041; A61K 49/0056; A61P 35/00; C07K 1/063; C07K 1/1075; C07K 14/00; A61K 38/00

(57) ABSTRACT

The disclosure relates to covalent protein dimers of MYC, MAX, and Omomyc; pharmaceutical compositions comprising the covalent protein dimers; methods of making the covalent protein dimers; and methods of treating disorders associated with MYC dysregulation (e.g., cancer) with the covalent protein dimers.

### CROSS REFERENCE TO RELATED APPLICATIONS

This application is a U.S. National Stage filing under 35 U.S.C. § 371 of International Patent Application No. PCT/US2022/033920, filed Jun. 17, 2022, which claims the priority benefit of U.S. Provisional Patent Application Ser. No. 63/213,024, filed Jun. 21, 2021. The entirety of these applications are hereby incorporated by reference.

### STATEMENT REGARDING FEDERALLY SPONSORED RESEARCH OR DEVELOPMENT

This invention was made with government support under Grant No. VR 2017-00372 awarded by the Swedish Research Council; Grant No. PO 2413/1-1, awarded by Deutsche Forschungsgemeinschaft; Grant No. 1122374 awarded by the National Science Foundation Graduate Research Fellowship; and Grant No. 174530 awarded by the National Science Foundation Graduate Research Fellowship. The government has certain rights in the invention.

### SEQUENCE LISTING

The instant application contains a Sequence Listing that has been submitted electronically in ASCII format and is hereby incorporated by reference in its entirety. Said ASCII file, created on Jul. 20, 2022, is named 20221025-021203-3.txt and is 3,901 bytes in size.

### BACKGROUND

The transcription factor protein MYC forms a heterodimer with MAX in order to bind to the E-Box DNA sequence (CACGTG). The MYC/MAX protein complex is part of the basic-helix-loop-helix/leucine-zipper (bHLH/Lz) transcription factor family and initiates several cellular processes, including cell proliferation and survival. MAX, alternatively, can homodimerize, compete for the E-Box DNA binding site, and inhibit MYC/MAX-driven transcription. MYC/MAX and MAX/MAX, thus, have opposite activities, and MYC overexpression is observed in >50% of human cancers.

Promising strategies to inhibit the oncogenic MYC activity rely on stabilizing the natural MAX/MAX dimer or delivering protein analogs with a similar mechanism of action. The targeting of MYC with small molecules has largely remained elusive, mainly because the structure of MYC presents no binding pockets for small molecule ligands. Recent attempts to overcome the challenge of drugging MYC include a small molecule stabilizer of the MAX/MAX complex that inhibits the proliferation of several cancer cell lines and reduces tumor burden in murine cancer models. An alternate approach involves the artificial miniprotein Omomyc, a dominant-negative form of MYC that can compete for E-Box DNA binding and inhibit MYC/MAX dependent transcription, ultimately resulting in tumor growth inhibition in various mouse models of cancer.

Omomyc, like MYC and MAX, has to form dimeric complexes to be functional and bioactive. MYC, MAX, and Omomyc can interact with each other in different combinations. Upon delivery of a monomer to the cell, the dominating complex formed depends on the other proteins' cellular concentrations and is difficult to predict. The direct administration of defined and stable dimeric complexes would offer a superior degree of control over the concentration and composition of the bioactive dimer inhibitor, in addition to a potentially higher structural stability.

Preparing homogeneous, stable, well-defined protein-protein conjugates can be a challenge. Chemical synthesis approaches to generate covalently linked multimeric proteins have been mainly focused on preparing ubiquitinylated or sumoylated proteins. These strategies relied on chemical ligation or chemoenzymatic workflows, requiring the incorporation of unnatural amino acids or engineered recognition sequences, respectively. In addition, ligation based strategies to prepare covalently linked HIV protease heterodimers have been reported with the aim to study asymmetric mutations of this enzyme dimer. Previous dimerization strategies of MYC/MAX analogs relied on either disulfide formation at the C-terminus of the leucine zipper region of the transcription factor analogs or the formation of oxime and thioester linkages between MYC and MAX or between MAX and MAX. While these defined dimers enabled DNA-binding studies, the reported strategies relied on dovetails with low chemical stability in a biological milieu, making them unsuitable for bioactivity studies in vivo. Accordingly, there is a need for biologically stable dimers of MYC, MAX, and Omomyc.

### SUMMARY

The present disclosure provides, inter alia, a covalent protein dimer, or a pharmaceutically acceptable salt thereof, comprising: a first polypeptide comprising a C-terminus and an N-terminus, wherein the first polypeptide comprises a degree of identity of at least 85% with respect to SEQ ID NO: 1, 2, or 3; a second polypeptide comprising a C-terminus and an N-terminus, wherein the second polypeptide comprises a degree of identity of at least 85% with respect to SEQ ID NO: 1, 2, or 3; and a linker covalently linking the C-terminus of the first polypeptide to the C-terminus of the second polypeptide.

In another aspect, the disclosure provides a covalent protein dimer, or a pharmaceutically acceptable salt thereof, having a structure according to Formula (I):

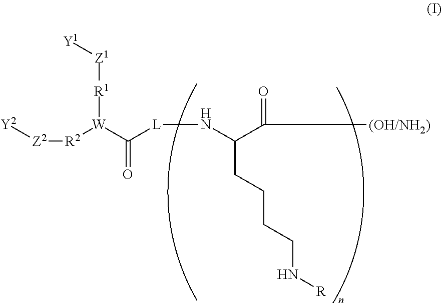

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
R¹ is absent, C₁₋₁₀ alkyl, or C₁₋₁₀ heteroalkyl;
R² is absent, C₁₋₁₀ alkyl, or C₁₋₁₀ heteroalkyl;
W is C₁₋₁₀ alkyl, C₁₋₁₀ heteroalkyl, C₆₋₁₀ aryl, or 5- to 10-membered heteroaryl;
L is absent or a linker;
R is H, a nitrogen protecting group, biotin, a fluorescent dye, a nuclear-targeting moiety, or a cell-penetrating moiety; and
n is 0 or 1.

In an embodiment, the covalent protein dimer has a structure according to Formula (Ib):

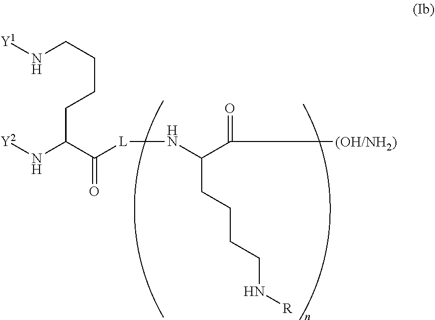

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety; and
n is 0 or 1.

In another aspect, the disclosure provides a covalent protein dimer, or a pharmaceutically acceptable salt thereof, having a structure according to Formula (II):

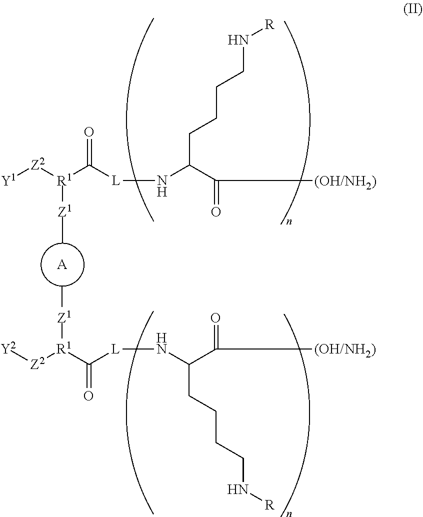

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ independently is —O—, —NH—, or —S—;
Z² independently is —O—, —NH—, or —S—;
R¹ independently is C₁₋₁₀ alkyl or C₁₋₁₀ heteroalkyl;
A is C₆₋₁₀ aryl or 5- to 10-membered heteroaryl;
L independently is absent or a linker;
R independently is H, a nitrogen protecting group, biotin, a fluorescent dye, a nuclear-targeting moiety, or a cell-penetrating moiety; and
n independently is 0 or 1.

In some embodiments, the covalent protein dimer has a structure according to Formula (IIb):

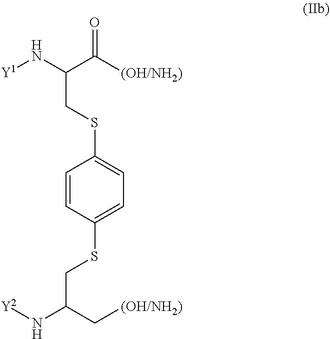

wherein:
Y¹ is a polypeptide comprising a degree of identity of at least 85% with respect to SEQ ID NO: 1, 2, or 3; and
Y² is a polypeptide comprising a degree of identity of at least 85% with respect to SEQ ID NO: 1, 2, or 3.

In another aspect, the disclosure provides a pharmaceutical composition comprising a covalent protein dimer of the disclosure and a pharmaceutically acceptable carrier. In another aspect, the disclosure provides a method of treating a disease or disorder characterized by MYC dysregulation in a subject in need thereof, the method comprising administering to the subject a covalent protein dimer of the disclosure. In an embodiment, the disease or disorder is cancer. In another aspect, the disclosure provides a method of making a covalent protein dimer of the disclosure.

### BRIEF DESCRIPTION OF THE DRAWINGS

FIG. 1 is a schematic of an automated flow protein synthesizer.

FIG. 2 is depiction of the synthesis time, yields, and LC-MS characterization of purified homodimers 3 and 4 and heterodimers 5 and 6. The panels show the total ion current chromatogram (TIC) as the base spectrum, the electrospray ionization (ESI) mass-to-charge spectra (left inset) and deconvoluted mass spectra (right inset).

FIG. 3 is a set of reaction schematics for 7, 8, and 9 along with TIC-LCMS chromatograms of the dimer conjugates with m/z and deconvoluted mass.

FIG. 4 is a set of flow cytometry histograms illustrating the dose-dependent increase in fluorescence of Hela cells after 15 min incubation with TAMRA-labeled dimers and Omomyc-TAMRA monomer at concentrations between 0.01 μM and 15 μM.

FIG. 5 is a set of micrographs from confocal microscopy; Hoechst (DAPI) labels the nuclei, and TAMRA-protein (Cy3) is observed throughout the cell after 15 min incubation, followed by incubation in fresh media for 1 h.

FIG. 6 is a gel showing synthetic protein dimers and monomers (˜1 μg per protein loaded). The bands were visualized by Coomassie blue staining.

FIG. 7 is a graph showing the results of circular dichroism analysis. Mean residual ellipticity (MRE) is shown as a function of wavelength for protein dimers dissolved in folding buffer.

FIG. 8 is a gel showing protein monomers and synthetic dimers incubated incubated with E-Box DNA in folding buffer. Samples were run on 10% polyacrylamide gel in TBE buffer and visualized with ethidium bromide.

FIG. 9 is a set of graphs showing differences in melting temperature between protein monomers and covalent dimers incubated with and without E-Box DNA.

FIG. 10 is a table showing the melting temperatures of various protein monomers and covalent dimers incubated with and without E-Box DNA.

FIG. 11 is a set of graphs depicting the results of cell proliferation assays of HeLa, A549 and H441 cells following treatment with covalent protein dimers for 72 h, quantified via CellTiter-Glo®.

FIG. 12 is a table summarizing the proliferation inhibition EC₅₀ values of the covalent protein

FIG. 13 is a graph showing the degree to which genes were upregulated or downregulated in A549 cells treated with 4. Upregulated genes with adjusted p-value <0.05 and |log2FC|≥1 are shown on the top half of the graph, downregulated genes with p-value <0.05 and |log2FC|≤1 are shown in the bottom half. Downregulated genes involved in KRas pathways are labeled.

FIG. 14 is an enrichment plot of MYC target gene signature showing a negative enrichment following exposure to 4 (q-value <0.05).

FIG. 15 is a schematic representation of the workflow to generate 10, 11, and 12.

FIG. 16 is a set of graphs depicting in-line UV₃₁₀ₙₘ monitoring for Fmoc-deprotection of 10, 11, and 12.

FIG. 17 is a set of LC-MS analysis and deconvoluted mass spectra of the crude analogs: C) Max 11; D) Myc 10; E) Omomyc 12; LC-MS analysis of purified analogs: F) Max 11; G) Myc 10; and H) Omomyc 12. The panels show the total ion current chromatogram (TIC) as the base spectrum, the electrospray ionization (ESI) mass-to-charge spectra (left inset) and deconvoluted mass spectra (right inset).

FIG. 18 is a schematic representation of all possible combinations of the proteins MYC, MAX, and Omomyc when the monomers are mixed in solution.

FIG. 19 is a gel depicting the electrophoretic mobility assay shift of dimeric analogs. Upward shifts of DNA bands indicate higher molecular weight (protein-DNA complex).

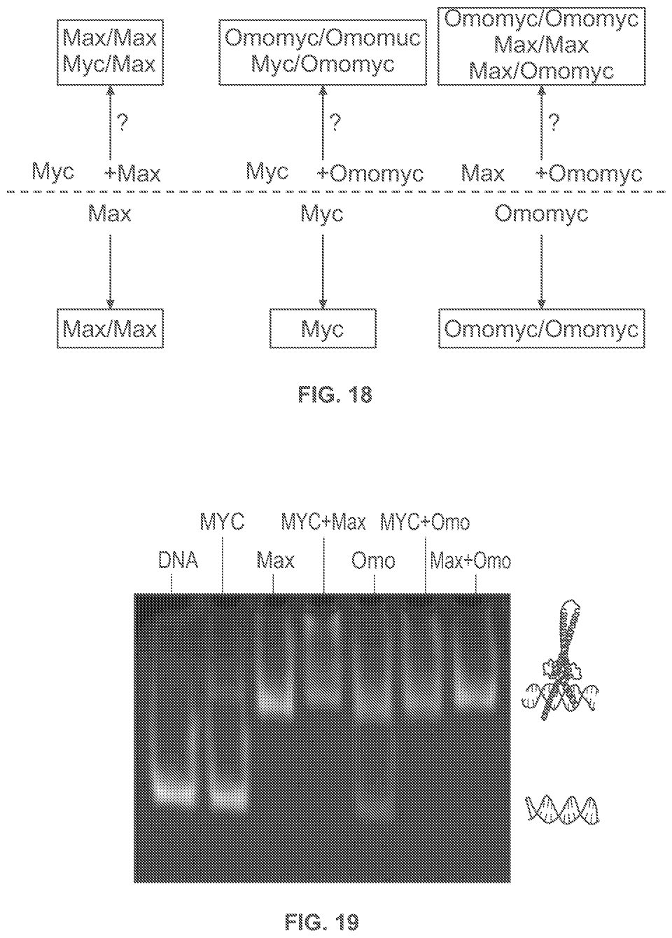

FIG. 18 is a schematic representation of all possible combinations of the proteins MYC, MAX, and Omomyc when the monomers are mixed in solution.

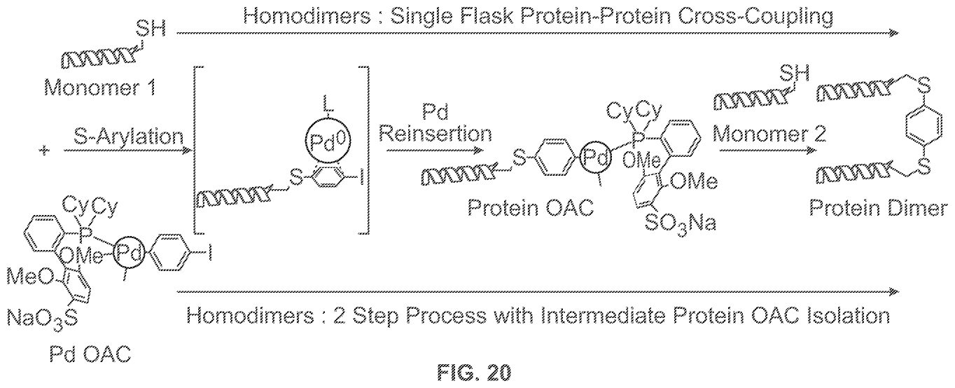

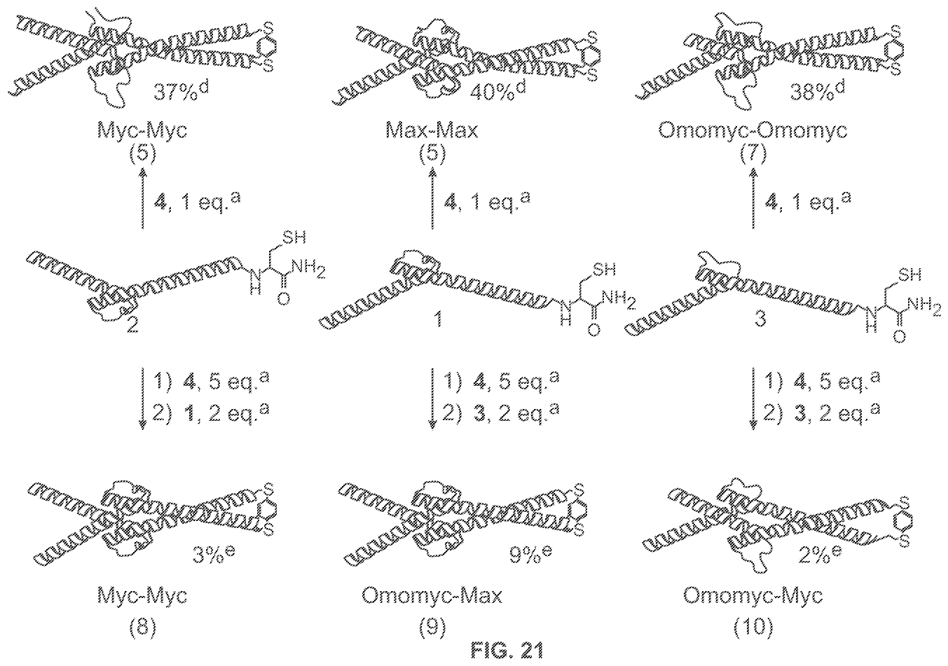

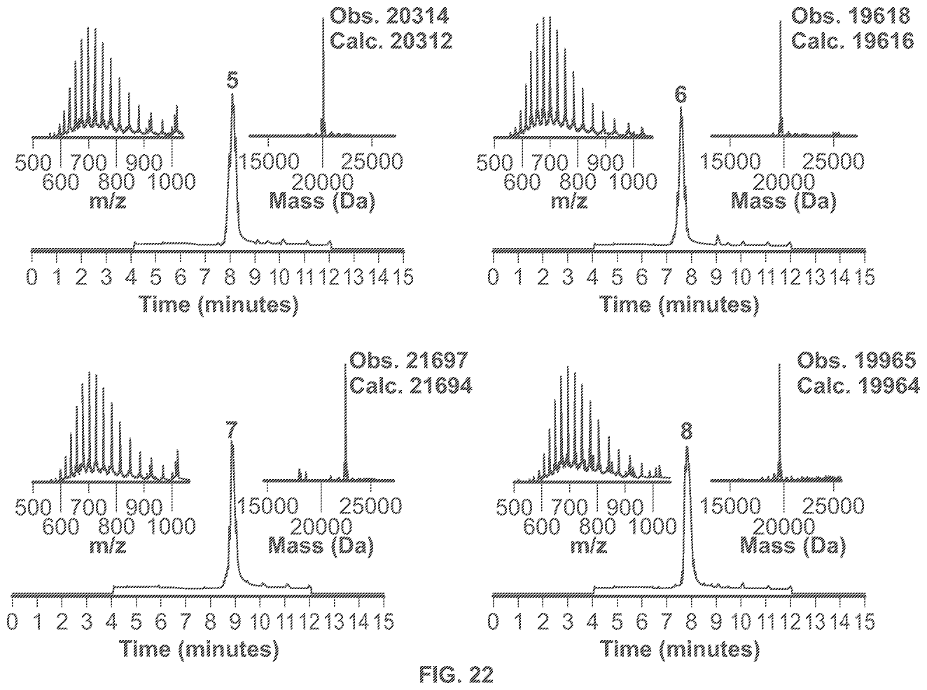

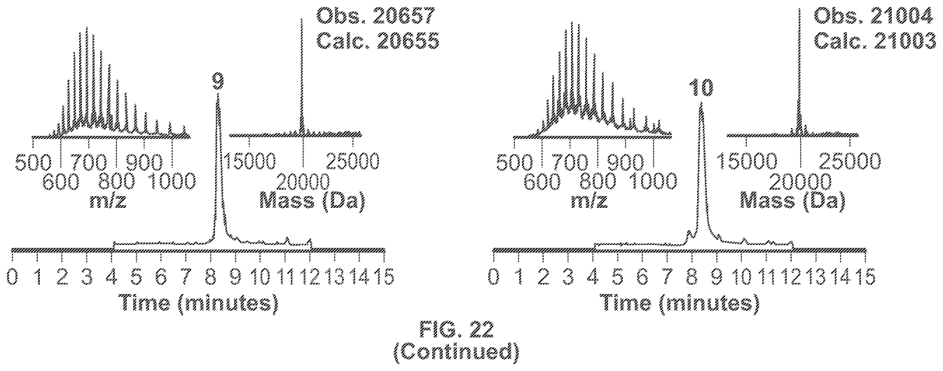

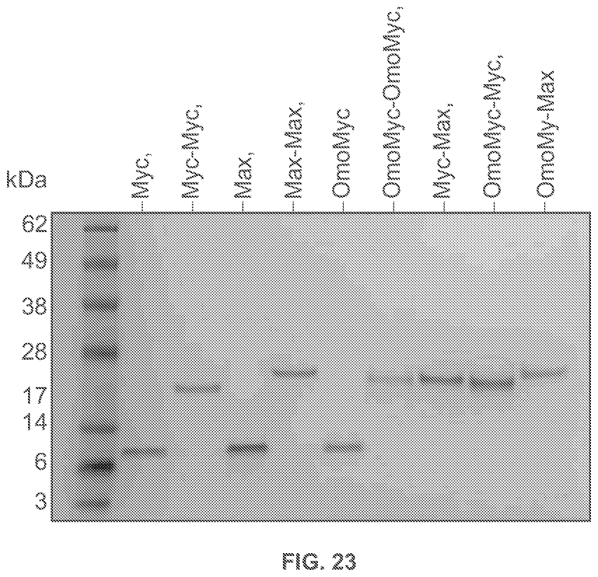

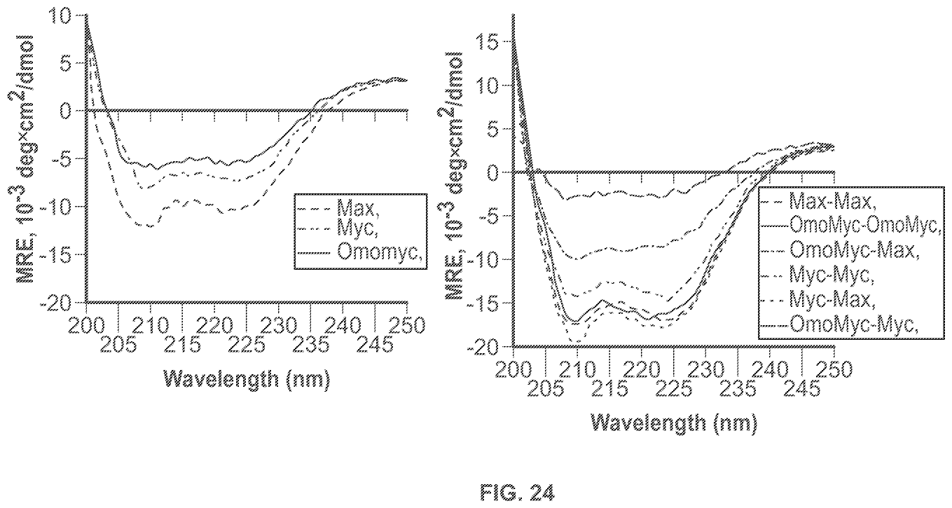

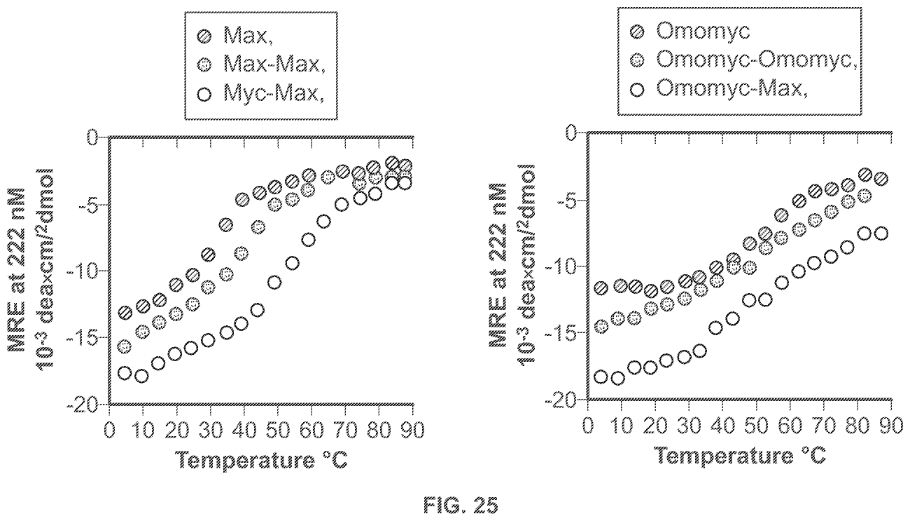

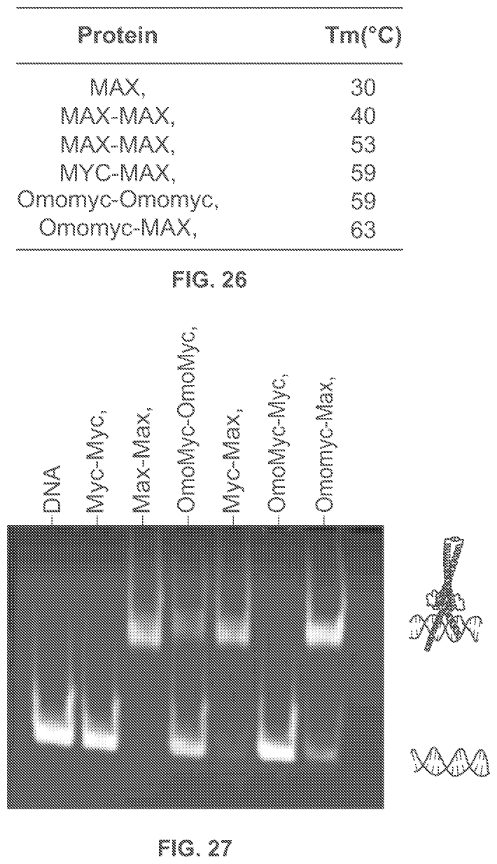

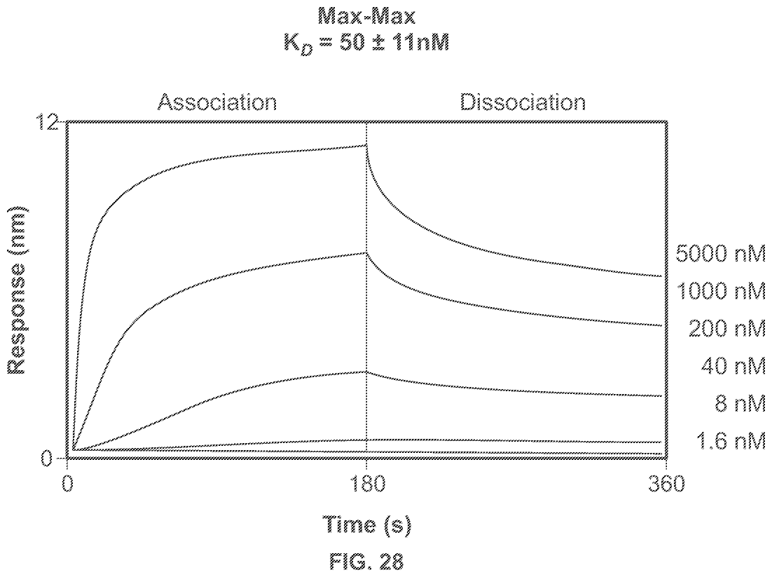

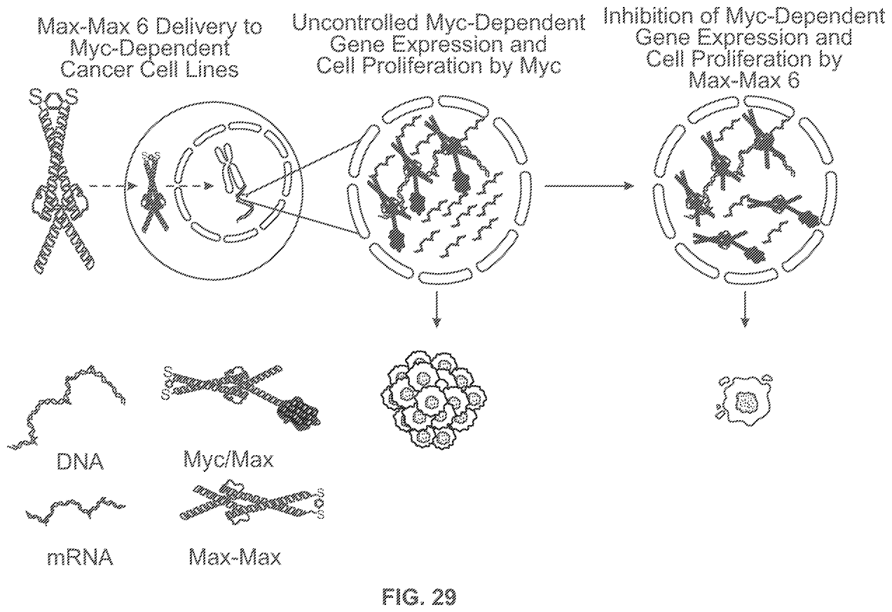

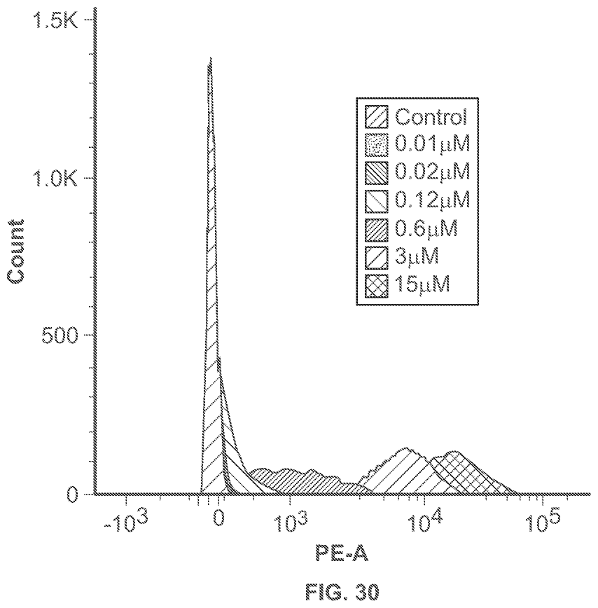

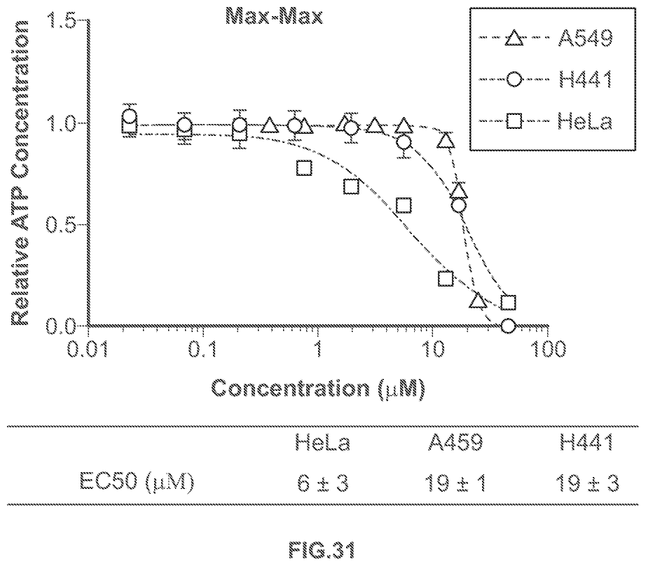

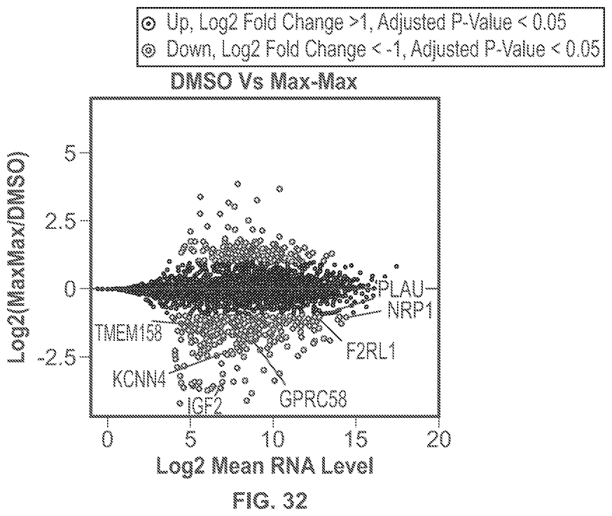

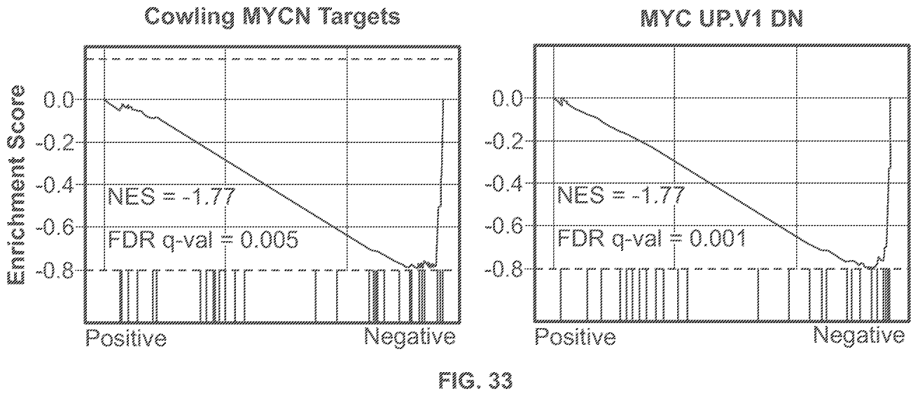

FIG. 19 is a gel depicting the electrophoretic mobility assay shift of dimeric analogs. Upward shifts of DNA bands indicate higher molecular weight (protein-DNA complex).

FIG. 20 is a schematic representation of the synthesis of the homo- and heterodimers using bifunctional Pd oxidative addition complexes (OACs).

FIG. 21 is a schematic representation of the protein-protein cross-coupling reactions using reagent Pd OAC (indicated as 4).

FIG. 22 is a series of deconvoluted mass spectra of the isolated covalent protein dimers 13, 14, 15, 16, 17, and 18, respectively. The panels show the total ion current chromatogram (TIC) as the base spectrum, the electrospray ionization (ESI) mass-to-charge spectra (left inset) and deconvoluted mass spectra (right inset).

FIG. 23 is an SDS-PAGE analysis of the monomeric protein analogs and the covalent protein dimers.

FIG. 24 is a set of graphs showing the circular dichroism analysis of the three monomeric analogs (left) and the six dimeric analogs 13, 14, 15, 16, 17, and 18 (right). The dimeric analogs exhibited alpha-helical patterns as displayed by the deep double minima at 207 nm and 222 nm.

FIG. 25 is a set of graphs showing mean residual elypticity (MRE) vs. temperature for 11, 14, 16, 12, 15, and 17.

FIG. 26 is a table of melting points for 11, 14, 16, 12, 15, and 17.

FIG. 27 is a gel depicting the electrophoretic mobility assay shift of dimeric analogs. Upward shifts of DNA bands indicate higher molecular weight (protein-DNA complex).

FIG. 28 is a sensorgram from a bio-layer interferometry analysis of Max-Max 14 binding to E-box DNA probe (K_{D}=50±11 nM).

FIG. 29 is a schematic representation of Max-Max delivery to Myc-dependent cancer cell lines to inhibit Myc.

FIG. 30 is a set of flow cytometry histograms illustrating the dose-dependent increase in fluorescence of Hela cells after 15 min incubation with 19.

FIG. 31 is a set of curves showing the decrease in ATP concentration in cancer cell lines treated with varying concentrations of Max-Max 14 for 72 h. ATP concentration is shown relative to untreated cells, determined by Cell-Titer Glo. Each point represents mean and standard deviation (n=3). Also shown are EC50 values of Max-Max 14 in HeLa, A549, and H441 cell lines, respectively.

FIG. 32 is a graph showing the degree to which genes were upregulated or downregulated in A549 cells treated with 14. Upregulated genes with adjusted p-value <0.05 and |log2FC|≤1 are shown on the top half of the graph, downregulated genes with p-value <0.05 and |log2FC|≤1 are shown in the bottom half. Downregulated genes involved in KRas pathways are labeled.

FIG. 33 is a set of enrichment plots of MYC target gene signature showing a negative enrichment following exposure to 14.

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with a subject an effective amount of a covalent protein dimer of the disclosure for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “MYC” refers to the protein MYC proto-oncogene encoded by the MYC gene, which is a member of the myc family of transcription factors, and has the following sequence:

(SEQ ID NO: 1)
NVKRRTHNVLERQRRNELKRSFFALRDQIPELENNEKAPKVVILKKATA
YILSVQAEEQKLISEEDLLRKRREQLKHKLEQLGG.

As used herein, the term “MAX” refers to the transcription factor myc-associated factor X, which is encoded by the MAX gene and has the following sequence:

(SEQ ID NO: 2)
DKRAHHNALERKRRDHIKDSFHSLRDSVPSLQGEKASRAQILDKATEYI
QYMRRKNHTHQQDIDDLKRQNALLEQQVRALGG.

As used herein, the term “Omomyc” refers the artificial mini-protein that functions as a dominant-negative form of MYC and has the following sequence:

(SEQ ID NO: 3)
MATEENVKRRTHNVLERQRRNELKRSFFALRDQIPELENNEKAPKVVIL
KKATAYILSVQAETQKLISEIDLLRKQNEQLKHKLEQLRNS.

As used herein the nomenclature “protein-protein” (e.g., MAX-MAX or MYC-MAX) indicates a covalent dimer whereas the nomenclature “protein/protein” (e.g., MAX/MAX or MYC/MAX) indicates a non-covalent dimer.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein “heteroalkyl” refers to an alkyl group wherein one or more carbon atoms has been replaced with a heteroatom selected from O, S, or N, wherein alkyl is as defined herein.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully or partially saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo [3.1.0] hexyl, spiro [3.3] heptanyl, and bicyclo [1.1.1] pentyl.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. Accordingly, the term “heterocyclyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2-pyrrolidinonyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo [2.1.1]hexanyl, 5-azabicyclo [2.1.1] hexanyl, 6-azabicyclo [3.1.1]heptanyl, 2-azabicyclo [2.2.1]heptanyl, 3-aza-bicyclo [3.1.1]heptanyl, 2-azabicyclo [3.1.1]heptanyl, 3-azabicyclo [3.1.0] hexanyl, 2-azabicyclo-[3.1.0]hexanyl, 3-azabicyclo [3.2.1]octanyl, 8-azabicyclo [3.2.1] octanyl, 3-oxa-7-azabicyclo [3.3.1]-nonanyl, 3-oxa-9-azabicyclo [3.3.1]nonanyl, 2-oxa-5-azabicyclo[2.2.1]heptanyl, 6-oxa-3-aza-bicyclo [3.1.1]heptanyl, 2-azaspiro[3.3]heptanyl, 2-oxa-6-azaspiro [3.3]heptanyl, 2-oxaspiro [3.3]-heptanyl, 2-oxaspiro[3.5]nonanyl, 3-oxaspiro [5.3]nonanyl, 2-azaspiro [3.3]heptane, 8-oxabicyclo[3.2.1]octanyl, 2,8-diazaspiro [4.5]decan-1-onyl, and 1,8-diazaspiro [4.5]decan-2-onyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo [1,2-a]pyridinyl, pyrazolo [1,5-a]pyridinyl, 5,6,7,8-tetrahydroisoquinolinyl, 5,6,7,8-tetrahydroquinolinyl, 6,7-dihydro-5H-cyclopenta [b]pyridinyl, 6,7-dihydro-5H-cyclo-penta[c]pyridinyl, 1,4,5,6-tetrahydrocyclopenta[c]pyrazolyl, 2,4,5,6-tetrahydrocyclopenta[c]-pyrazolyl, 5,6-dihydro-4H-pyrrolo [1,2-b]pyrazolyl, 6,7-dihydro-5H-pyrrolo [1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl, heteroaryl, cycloalkyl, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the phrase “protecting group” refers to a functional group introduced into a molecule by chemical modification of an oxygen atom, a nitrogen atom, or a sulfur atom to obtain chemoselectivity in a subsequent chemical reaction. Examples of hydroxyl protecting groups include, but are not limited to methoxymethyl (MOM), tetrahydropyranyl (THP), allyl, benzyl (Bn), tert-butyldimethylsilyl (TBDMS), pivaloyl (Piv), and benzoyl (Bz). Examples of nitrogen protecting groups include, but are not limited to, allyloxycarbonyl (Alloc), carbobenzyloxy (Cbz), tert-butyloxycarbonyl (Boc), 9-fluorenylmethyloxycarbonyl (Fmoc), acetyl (Ac), benzoyl (Bz), tosyl (Ts), and benzyl (Bn). Examples of sulfur protecting groups include, but are not limited to methoxymethyl (MOM), allyl, trityl (Trt), trichloroacetyl, pivaloyl (Piv), and benzoyl (Bz).

As used herein, the term “optionally substituted” means that the referenced group may be substituted or unsubstituted. As used herein, the term “substituted” means that an atom or group of atoms has replaced hydrogen as the substituent attached to another group.

### Covalent Protein Dimers

Provided herein are covalent protein dimers that inhibit the activity of the MYC/MAX complex, which are useful in the treatment of MYC-related disorders, including cancer and other proliferation diseases.

In an aspect, provided herein is a covalent protein dimer, or a pharmaceutically acceptable salt thereof, comprising:
a first polypeptide comprising a C-terminus and an N-terminus, wherein the first polypeptide comprises a degree of identity of at least 85% with respect to SEQ ID NO: 1, 2, or 3;
a second polypeptide comprising a C-terminus and an N-terminus, wherein the second polypeptide comprises a degree of identity of at least 85% with respect to SEQ ID NO: 1, 2, or 3; and
a linker covalently linking the C-terminus of the first polypeptide to the C-terminus of the second polypeptide.

In some embodiments, the first polypeptide comprises a degree of identity of at least 90% with respect to SEQ ID NO: 1, 2, or 3. In some embodiments, the first polypeptide comprises a degree of identity of at least 95% with respect to SEQ ID NO: 1, 2, or 3. In some embodiments, the first polypeptide comprises a sequence represented by SEQ ID NO: 1, 2, or 3.

In some embodiments, the second polypeptide comprises a degree of identity of at least 90% with respect to SEQ ID NO: 1, 2, or 3. In some embodiments, the second polypeptide comprises a degree of identity of at least 95% with respect to SEQ ID NO: 1, 2, or 3. In some embodiments, the second polypeptide comprises a sequence represented by SEQ ID NO: 1, 2, or 3.

In some embodiments, the first polypeptide is at least 85% identical to SEQ ID NO: 2, and the second polypeptide is at least 85% identical to SEQ ID NO: 2; the first polypeptide is at least 85% identical to SEQ ID NO: 3, and the second polypeptide is at least 85% identical to SEQ ID NO: 3; the first polypeptide is at least 85% identical to SEQ ID NO: 1, and the second polypeptide is at least 85% identical to SEQ ID NO: 2; or the first polypeptide is at least 85% identical to SEQ ID NO: 3, and the second polypeptide is at least 85% identical to SEQ ID NO: 2.

In some embodiments, the first polypeptide is at least 85% identical to SEQ ID NO: 2, and the second polypeptide is at least 85% identical to SEQ ID NO: 2. In some embodiments, the first polypeptide is at least 90% identical to SEQ ID NO: 2, and the second polypeptide is at least 90% identical to SEQ ID NO: 2. In some embodiments, the first polypeptide is at least 95% identical to SEQ ID NO: 2, and the second polypeptide is at least 95% identical to SEQ ID NO: 2. In some embodiments, the first polypeptide comprises a sequence represented by SEQ ID NO: 2, and the second polypeptide comprises a sequence represented by SEQ ID NO: 2.

In some embodiments, the first polypeptide is at least 85% identical to SEQ ID NO: 3, and the second polypeptide is at least 85% identical to SEQ ID NO: 3. In some embodiments, the first polypeptide is at least 90% identical to SEQ ID NO: 3, and the second polypeptide is at least 90% identical to SEQ ID NO: 3. In some embodiments, the first polypeptide is at least 95% identical to SEQ ID NO: 3, and the second polypeptide is at least 95% identical to SEQ ID NO: 3. In some embodiments, the first polypeptide comprises a sequence represented by SEQ ID NO: 3, and the second polypeptide comprises a sequence represented by SEQ ID NO: 3.

In some embodiments, the first polypeptide is at least 85% identical to SEQ ID NO: 1, and the second polypeptide is at least 85% identical to SEQ ID NO: 2. In some embodiments, the first polypeptide is at least 90% identical to SEQ ID NO: 1, and the second polypeptide is at least 90% identical to SEQ ID NO: 2. In some embodiments, the first polypeptide is at least 95% identical to SEQ ID NO: 1, and the second polypeptide is at least 95% identical to SEQ ID NO: 2. In some embodiments, the first polypeptide comprises a sequence represented by SEQ ID NO: 1, and the second polypeptide comprises a sequence represented by SEQ ID NO: 2.

In some embodiments, the first polypeptide is at least 85% identical to SEQ ID NO: 3, and the second polypeptide is at least 85% identical to SEQ ID NO: 2. In some embodiments, the first polypeptide is at least 90% identical to SEQ ID NO: 3, and the second polypeptide is at least 90% identical to SEQ ID NO: 2. In some embodiments, the first polypeptide is at least 95% identical to SEQ ID NO: 3, and the second polypeptide is at least 95% identical to SEQ ID NO: 2. In some embodiments, the first polypeptide comprises a sequence represented by SEQ ID NO: 3, and the second polypeptide comprises a sequence represented by SEQ ID NO: 2.

In general, the linker is a chemical moiety comprising a covalent bond or a chain of atoms that covalently attaches the C-terminus of the first polypeptide to the C-terminus of the second polypeptide. Exemplary linkers may comprise at least one optionally substituted; saturated or unsaturated; linear, branched or cyclic alkyl group or an optionally substituted aryl group. The linker may also be a polypeptide (e.g., from about 1 to about 50 amino acids or more, or from about 1 to about 5 amino acids). In some embodiments, the linker is biologically stable and is not readily cleavable under physiological environments or conditions.

In another aspect, provided herein is a covalent protein dimer, or a pharmaceutically acceptable salt thereof, having a structure according to Formula (I):

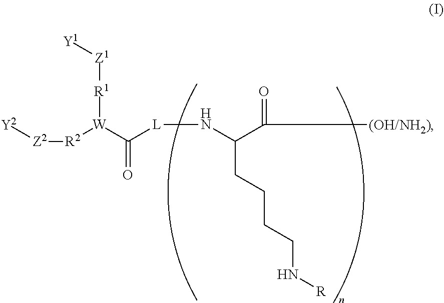

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
R¹ is absent, C₁₋₁₀ alkyl, or C₁₋₁₀ heteroalkyl;
R² is absent, C₁₋₁₀ alkyl, or C₁₋₁₀ heteroalkyl;
W is C₁₋₁₀ alkyl, C₁₋₁₀ heteroalkyl, C₆₋₁₀ aryl, or 5- to 10-membered heteroaryl;
L is absent or a linker;
R is H, a nitrogen protecting group, biotin, a fluorescent dye, a nuclear-targeting moiety, or a cell-penetrating moiety; and
n is 0 or 1.

In some embodiments, the covalent protein dimer of Formula (I) has a structure according to Formula (Ia):

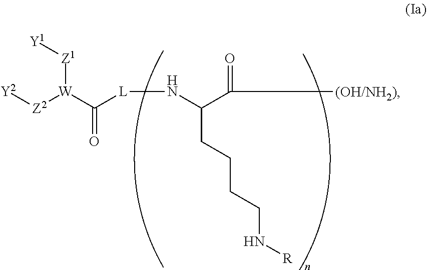

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
W is C₁₋₁₀ alkyl or C₁₋₁₀ heteroalkyl;
L is absent or a linker;
R is H, a protecting group, a fluorescent dye, biotin, a nuclear-targeting moiety, or a cell-penetrating moiety; and
n is 0 or 1.

In some embodiments, the covalent protein dimer of Formula (I) has a structure according to Formula (Ib):

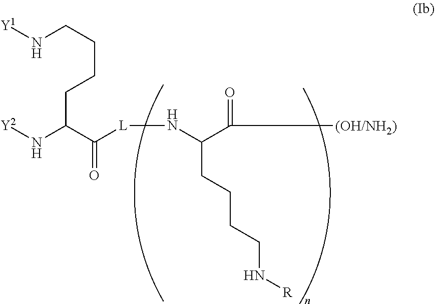

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety; and
n is 0 or 1.

In some embodiments, Y¹ comprises a C-terminus and an N-terminus, wherein the C-terminus forms a bond with Z¹ or —NH—.

In some embodiments, Y² comprises a C-terminus and an N-terminus, wherein the C-terminus forms a bond with Z² or —NH—.

In some embodiments, if one of Y¹ or Y² is at least 85% identical to SEQ ID NO: 1, then the other is at least 85% identical to SEQ ID NO: 2.

In some embodiments, Y¹ is at least 90% identical to SEQ ID NO: 1, 2, or 3. In some embodiments, Y¹ is at least 95% identical to SEQ ID NO: 1, 2, or 3. In some embodiments, Y¹ comprises a sequence represented by SEQ ID NO: 1, 2, or 3.

In some embodiments, Y² is at least 90% identical to SEQ ID NO: 1, 2, or 3. In some embodiments, Y² is at least 95% identical to SEQ ID NO: 1, 2, or 3. In some embodiments, Y² comprises a sequence represented by SEQ ID NO: 1, 2, or 3.

In some embodiments, Y¹ is at least 85% identical to SEQ ID NO: 2, and Y² is at least 85% identical to SEQ ID NO: 2;Y¹ is at least 85% identical to SEQ ID NO: 3, and Y² is at least 85% identical to SEQ ID NO: 3; Y¹ is at least 85% identical to SEQ ID NO: 1, and Y² is at least 85% identical to SEQ ID NO: 2; or Y¹ is at least 85% identical to SEQ ID NO: 3, and Y² is at least 85% identical to SEQ ID NO: 2.

In some embodiments, Y¹ is at least 85% identical to SEQ ID NO: 2, and Y² is at least 85% identical to SEQ ID NO: 2. In some embodiments, Y¹ is at least 90% identical to SEQ ID NO: 2,and Y² is at least 90% identical to SEQ ID NO: 2. In some embodiments, Y¹ is at least 95% identical to SEQ ID NO: 2, and Y² is at least 95% identical to SEQ ID NO: 2. In some embodiments, Y¹ comprises a sequence represented by SEQ ID NO: 2, and Y² comprises a sequence represented by SEQ ID NO: 2.

In some embodiments, Y¹ is at least 85% identical to SEQ ID NO: 3, and Y² is at least 85% identical to SEQ ID NO: 3. In some embodiments, Y¹ is at least 90% identical to SEQ ID NO: 3, and Y² is at least 90% identical to SEQ ID NO: 3. In some embodiments, Y¹ is at least 95% identical to SEQ ID NO: 3, and Y² is at least 95% identical to SEQ ID NO: 3. In some embodiments, Y¹ comprises a sequence represented by SEQ ID NO: 3, and Y² comprises a sequence represented by SEQ ID NO: 3.

In some embodiments, Y¹ is at least 85% identical to SEQ ID NO: 1, and Y² is at least 85% identical to SEQ ID NO: 2. In some embodiments, Y¹ is at least 90% identical to SEQ ID NO: 1, and Y² is at least 90% identical to SEQ ID NO: 2. In some embodiments, Y¹ is at least 95% identical to SEQ ID NO: 1,and Y² is at least 95% identical to SEQ ID NO: 2. In some embodiments, Y¹ comprises a sequence represented by SEQ ID NO: 1, and Y² comprises a sequence represented by SEQ ID NO: 2.

In some embodiments, Y¹ is at least 85% identical to SEQ ID NO: 3, and Y2 is at least 85% identical to SEQ ID NO: 2. In some embodiments, Y¹ is at least 90% identical to SEQ ID NO: 3, and Y² is at least 90% identical to SEQ ID NO: 2. In some embodiments, Y¹ is at least 95% identical to SEQ ID NO: 3, and Y² is at least 95% identical to SEQ ID NO: 2. In some embodiments, Y¹ comprises a sequence represented by SEQ ID NO: 3, and Y² comprises a sequence represented by SEQ ID NO: 2.

In some embodiments, Z¹ is —NH—. In some embodiments, Z² is —NH—.

In some embodiments, R¹ is absent or C₁₋₁₀ alkyl. In some embodiments, R² is absent or C₁₋₁₀ alkyl.

In some embodiments, W is C₁₋₁₀ alkyl or C₁₋₁₀ heteroalkyl. In some embodiments, W is C₁₋₅ alkyl or C₁₋₅ heteroalkyl.

In some embodiments, L is absent.

In some embodiments, L is a linker. Exemplary linkers may comprise at least one optionally substituted; saturated or unsaturated; linear, branched or cyclic alkyl group or an optionally substituted aryl group. In some embodiments, the linker is a polypeptide. In some embodiments, the linker comprises one to fifty amino acids. In some embodiments, the linker comprises one to twenty-five amino acids. In some embodiments, the linker comprises one to ten amino acids. In some embodiments, the linker comprises one to five amino acids. In some embodiments, L is β-alanine.

In some embodiments, R is H.

In some embodiments, R is a nitrogen protecting group that is not 9-fluorenylmethyloxycarbonyl (Fmoc). In some embodiments, R is a nitrogen protecting group selected from the group consisting of allyloxycarbonyl (Alloc), carbobenzyloxy (Cbz), tert-butyloxycarbonyl (Boc), acetyl (Ac), benzoyl (Bz), tosyl (Ts), and benzyl (Bn). In some embodiments, R is Alloc or Boc.

In some embodiments, R is a fluorescent dye. Fluorescent dyes suitable for the covalent protein dimers include any fluorescent dye known in the art that may be covalently linked to dimer by way of the nitrogen atom adjacent variable R. Non-limiting examples of fluorescent dyes include Alexa Fluor fluorescent dyes, DyLight Fluor fluorescent dyes, rhodamine dyes, blue fluorescent protein (BFP), cyan fluorescent protein (CFP), green fluorescent protein (GFP), enhanced green fluorescent protein (eGFP), Cascade Blue™, Marina Blue™, Pacific Orange™, Oregon Green™, Cascade Yellow™, BODIPY, coumarin, methoxycoumarin, aminomethylcoumarin (AMCA), dansyl, 5-TAMRA, fluorescein, mBanana, mOrange, mHoneydew, mTangerine, mCherry, and mPlum. In some embodiments, the fluorescent dye is 5-TAMRA.

In some embodiments, R is a nuclear targeting moiety. In some embodiments, R is Mach3 having the sequence:

(SEQ ID NO: 4)
QKKRKSKANKKNWPKGKLSIHAKDYKQGPKAKXₐₐRKQRXₐₐRG,

wherein Xₐₐ is 6-aminohexanoic acid.

In some embodiments, n is 0. In some embodiments, n is 1.

In another aspect, provided herein is a covalent protein dimer, or a pharmaceutically acceptable salt thereof, having a structure according to Formula (II):

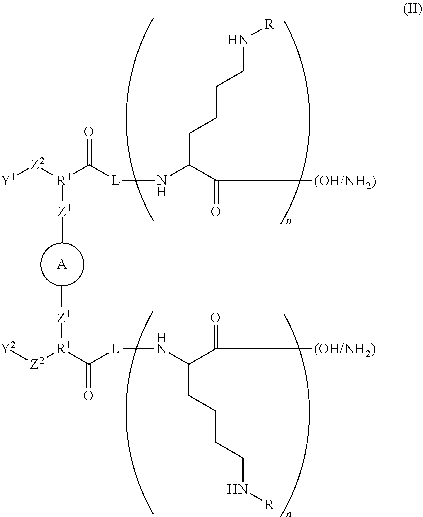

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ independently is —O—, —NH—, or —S—;
Z² independently is —O—, —NH—, or —S—;
R¹ independently is C₁₋₁₀ alkyl or C₁₋₁₀ heteroalkyl;
A is C₆₋₁₀ aryl or 5- to 10-membered heteroaryl;
L independently is absent or a linker;
R independently is H, a nitrogen protecting group, biotin, a fluorescent dye, a nuclear-targeting moiety, or a cell-penetrating moiety; and
n independently is 0 or 1.

In some embodiments, the covalent protein dimer of Formula (II) has a structure according to Formula (IIa):

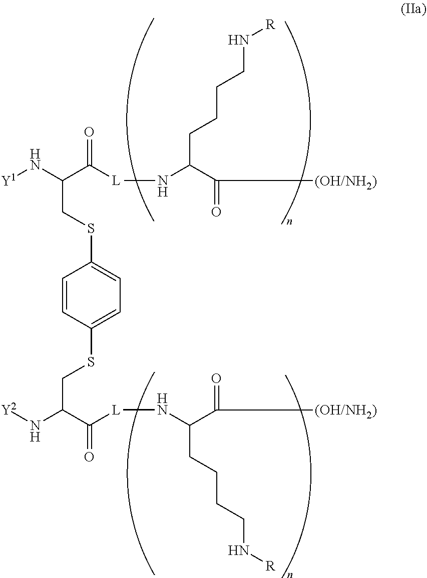

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L independently is absent or a linker;
R independently is H, a nitrogen protecting group, biotin, a fluorescent dye, a nuclear-targeting moiety, or a cell-penetrating moiety; and
n independently is 0 or 1.

In some embodiments, the covalent protein dimer of Formula (II) has a structure according to Formula (IIb):

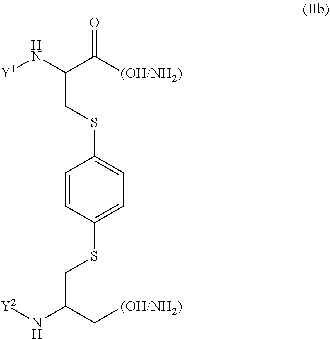

wherein:
Y¹ is a polypeptide comprising a degree of identity of at least 85% with respect to SEQ ID NO: 1, 2, or 3; and
Y² is a polypeptide comprising a degree of identity of at least 85% with respect to SEQ ID NO: 1, 2, or 3.

In some embodiments, Y¹ comprises a C-terminus and an N-terminus, wherein the C-terminus forms a bond with Z² or —NH—.

In some embodiments, Y² comprises a C-terminus and an N-terminus, wherein the C-terminus forms a bond with Z² or —NH—.

In some embodiments, Y¹ is at least 90% identical to SEQ ID NO: 1, 2, or 3. In some embodiments, Y¹ is at least 95% identical to SEQ ID NO: 1, 2, or 3. In some embodiments, Y¹ comprises a sequence represented by SEQ ID NO: 1, 2, or 3.

In some embodiments, Y² is at least 90% identical to SEQ ID NO: 1, 2, or 3. In some embodiments, Y² is at least 95% identical to SEQ ID NO: 1, 2, or 3. In some embodiments, Y² comprises a sequence represented by SEQ ID NO: 1, 2, or 3.

In some embodiments, Y¹ is at least 85% identical to SEQ ID NO: 2, and Y² is at least 85% identical to SEQ ID NO: 2; Y¹ is at least 85% identical to SEQ ID NO: 3, and Y² is at least 85% identical to SEQ ID NO: 3; Y¹ is at least 85% identical to SEQ ID NO: 1, and Y² is at least 85% identical to SEQ ID NO: 1; Y¹ is at least 85% identical to SEQ ID NO: 1, and Y² is at least 85% identical to SEQ ID NO: 2; Y¹ is at least 85% identical to SEQ ID NO: 3, and Y² is at least 85% identical to SEQ ID NO: 2; or Y¹ is at least 85% identical to SEQ ID NO: 3, and Y² is at least 85% identical to SEQ ID NO: 1;.

In some embodiments, Y¹ is at least 85% identical to SEQ ID NO: 2, and Y² is at least 85% identical to SEQ ID NO: 2. In some embodiments, Y¹ is at least 90% identical to SEQ ID NO: 2, and Y² is at least 90% identical to SEQ ID NO: 2. In some embodiments, Y¹ is at least 95% identical to SEQ ID NO: 2, and Y² is at least 95% identical to SEQ ID NO: 2. In some embodiments, Y¹ comprises a sequence represented by SEQ ID NO: 2, and Y² comprises a sequence represented by SEQ ID NO: 2.

In some embodiments, Y¹ is at least 85% identical to SEQ ID NO: 3, and Y² is at least 85% identical to SEQ ID NO: 3. In some embodiments, Y¹ is at least 90% identical to SEQ ID NO: 3, and Y² is at least 90% identical to SEQ ID NO: 3. In some embodiments, Y¹ is at least 95% identical to SEQ ID NO: 3, and Y² is at least 95% identical to SEQ ID NO: 3. In some embodiments, Y¹ comprises a sequence represented by SEQ ID NO: 3, and Y² comprises a sequence represented by SEQ ID NO: 3.

In some embodiments, Y¹ is at least 85% identical to SEQ ID NO: 1, and Y² is at least 85% identical to SEQ ID NO: 1. In some embodiments, Y¹ is at least 90% identical to SEQ ID NO: 1, and Y² is at least 90% identical to SEQ ID NO: 1. In some embodiments, Y¹ is at least 95% identical to SEQ ID NO: 1, and Y² is at least 95% identical to SEQ ID NO: 1. In some embodiments, Y¹ comprises a sequence represented by SEQ ID NO: 1, and Y² comprises a sequence represented by SEQ ID NO: 1.

In some embodiments, Y¹ is at least 85% identical to SEQ ID NO: 1, and Y² is at least 85% identical to SEQ ID NO: 2. In some embodiments, Y¹ is at least 90% identical to SEQ ID NO: 1, and Y² is at least 90% identical to SEQ ID NO: 2. In some embodiments, Y¹ is at least 95% identical to SEQ ID NO: 1, and Y² is at least 95% identical to SEQ ID NO: 2. In some embodiments, Y¹ comprises a sequence represented by SEQ ID NO: 1, and Y² comprises a sequence represented by SEQ ID NO: 2.

In some embodiments, Y¹ is at least 85% identical to SEQ ID NO: 3, and Y² is at least 85% identical to SEQ ID NO: 2. In some embodiments, Y¹ is at least 90% identical to SEQ ID NO: 3, and Y² is at least 90% identical to SEQ ID NO: 2. In some embodiments, Y¹ is at least 95% identical to SEQ ID NO: 3, and Y² is at least 95% identical to SEQ ID NO: 2. In some embodiments, Y¹ comprises a sequence represented by SEQ ID NO: 3, and Y² comprises a sequence represented by SEQ ID NO: 2.

In some embodiments, Y¹ is at least 85% identical to SEQ ID NO: 3, and Y² is at least 85% identical to SEQ ID NO: 1. In some embodiments, Y¹ is at least 90% identical to SEQ ID NO: 3, and Y² is at least 90% identical to SEQ ID NO: 1. In some embodiments, Y¹ is at least 95% identical to SEQ ID NO: 3, and Y² is at least 95% identical to SEQ ID NO: 1. In some embodiments, Y¹ comprises a sequence represented by SEQ ID NO: 3, and Y² comprises a sequence represented by SEQ ID NO: 1.

In some embodiments, Z¹ is —S—. In some embodiments, Z² is —NH—.

In some embodiments, R¹ is C₁₋₁₀ alkyl. In some embodiments, R¹ is C₁₋₅ alkyl or C₁₋₅ heteroalkyl.

In some embodiments, A is C₆₋₁₀ aryl. In some embodiments, A is 5- to 10-membered heteroaryl. In some embodiments, A is phenyl or 5- to 6-membered heteroaryl. In some embodiments, A is phenyl.

In some embodiments, L is absent.

In some embodiments, L is a linker. Exemplary linkers may comprise at least one optionally substituted; saturated or unsaturated; linear, branched or cyclic alkyl group or an optionally substituted aryl group. In some embodiments, the linker is a polypeptide. In some embodiments, the linker comprises one to fifty amino acids. In some embodiments, the linker comprises one to twenty-five amino acids. In some embodiments, the linker comprises one to ten amino acids. In some embodiments, the linker comprises one to five amino acids. In some embodiments, L is β-alanine.

In some embodiments, R is a nitrogen protecting group that is not 9-fluorenylmethyloxycarbonyl (Fmoc). In some embodiments, R is a nitrogen protecting group selected from the group consisting of allyloxycarbonyl (Alloc), carbobenzyloxy (Cbz), tert-butyloxycarbonyl (Boc), acetyl (Ac), benzoyl (Bz), tosyl (Ts), and benzyl (Bn). In some embodiments, R is Alloc or Boc.

In some embodiments, R is a fluorescent dye. Fluorescent dyes suitable for the covalent protein dimers include any fluorescent dye known in the art that may be covalently linked to dimer by way of the nitrogen atom adjacent variable R. Non-limiting examples of fluorescent dyes include Alexa Fluor fluorescent dyes, DyLight Fluor fluorescent dyes, rhodamine dyes, blue fluorescent protein (BFP), cyan fluorescent protein (CFP), green fluorescent protein (GFP), enhanced green fluorescent protein (eGFP), Cascade Blue™, Marina Blue™, Pacific Orange™, Oregon Green™, Cascade Yellow™, BODIPY, coumarin, methoxycoumarin, aminomethylcoumarin (AMCA), dansyl, 5-TAMRA, fluorescein, mBanana, mOrange, mHoneydew, mTangerine, mCherry, and mPlum. In some embodiments, the fluorescent dye is 5-TAMRA.

In some embodiments, R is a nuclear targeting moiety. In some embodiments, R is Mach3 having the sequence:

(SEQ ID NO: 4)
QKKRKSKANKKNWPKGKLSIHAKDYKQGPKAKXₐₐRKQRXₐₐRG,

wherein Xₐₐ is 6-aminohexanoic acid.

In some embodiments, n is 0. In some embodiments, n is 1.

The covalent protein dimers disclosed herein may exist as tautomers and optical isomers (e.g., enantiomers, diastereomers, diastereomeric mixtures, racemic mixtures, and the like).

In an aspect, provided herein is a pharmaceutical composition comprising a covalent protein dimer disclosed herein and a pharmaceutically acceptable carrier.

In an embodiment, the pharmaceutical compositions described herein include a therapeutically or prophylactically effective amount of a compound described herein. The pharmaceutical composition may be useful for treating a proliferative disease in a subject in need thereof, preventing a proliferative disease in a subject in need thereof, or inhibiting the activity of MYC in a subject, biological sample, tissue, or cell. In some embodiments, the proliferative disease is cancer.

### Synthesis of Covalent Protein Dimers

Also provided herein are methods of making the covalent protein dimers disclosed herein. Accordingly, in an aspect, the disclosure provides a method of making a covalent protein dimer, or a pharmaceutically acceptable salt thereof, having a structure according to Formula (I):

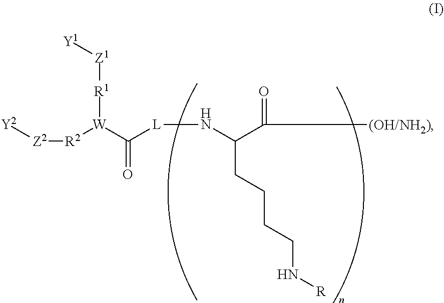

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
R¹ is absent, C₁₋₁₀ alkyl, or C₁₋₁₀ heteroalkyl;
R² is absent, C₁₋₁₀ alkyl, or C₁₋₁₀ heteroalkyl;
W is C₁₋₁₀ alkyl, C₁₋₁₀ heteroalkyl, C₆₋₁₀ aryl, or 5- to 10-membered heteroaryl;
L is absent or a linker;
R is H, a nitrogen protecting group, biotin, a fluorescent dye, a nuclear-targeting moiety, or a cell-penetrating moiety; and
n is 0 or 1;
the method comprising:
(a) reacting a first resin-bound, side-chain-protected peptide having a structure according to Formula (III):

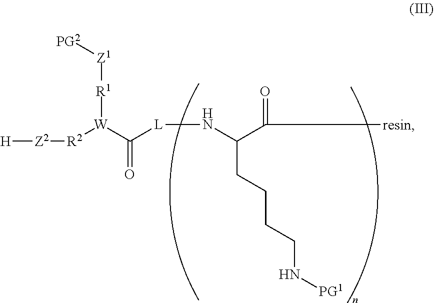

with one or more amino acids corresponding to the amino acids of the polypeptide represented by Y² to provide a second resin-bound, side-chain-protected peptide having a structure according to Formula (IV):

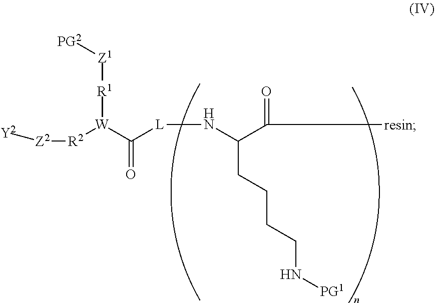

wherein PG¹ and PG² are non-identical protecting groups, and wherein neither PG¹ nor PG² are Fmoc;
(b) removing PG² from the second resin-bound, side-chain-protected peptide to provide a third resin-bound, side-chain-protected peptide having a structure according to Formula (V):

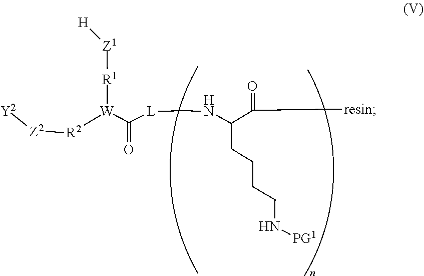

(c) reacting the third resin-bound, side-chain-protected peptide with one or more amino acids corresponding to the amino acids of the polypeptide represented by Y¹ to provide a fourth resin-bound, side-chain protected peptide having a structure according to Formula (VI):

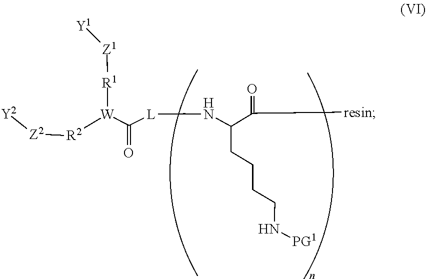

and
(d) cleaving the fourth resin-bound, side-chain-protected peptide from the resin to provide the covalent protein dimer.

In some embodiments, the covalent protein dimer of Formula (I) has a structure according to Formula (Ia):

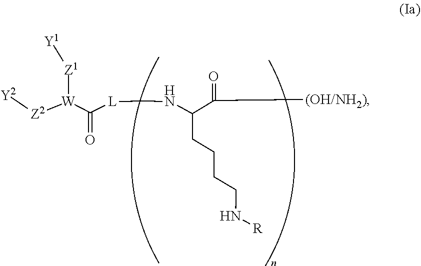

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
W is C₁₋₁₀ alkyl or C₁₋₁₀ heteroalkyl;
L is absent or a linker;
R is H, a protecting group, a fluorescent dye, biotin, a nuclear-targeting moiety, or a cell-penetrating moiety; and
n is 0 or 1;
the method comprising:
(a) reacting a first resin-bound, side-chain-protected peptide having a structure according to Formula (IIIa):

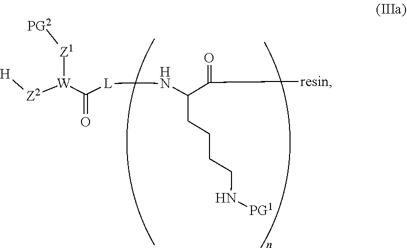

with one or more amino acids corresponding to the amino acids of the polypeptide represented by Y² to provide a second resin-bound, side-chain-protected peptide having a structure according to Formula (IVa):

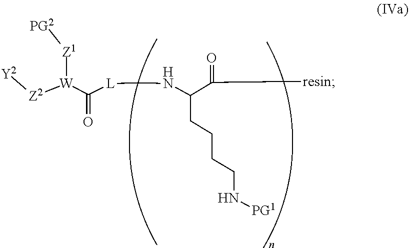

wherein PG¹ and PG² are non-identical protecting groups, and wherein neither PG¹ nor PG² are Fmoc;
(b) removing PG² from the second resin-bound, side-chain-protected peptide to provide a third resin-bound, side-chain-protected peptide having a structure according to Formula (Va):

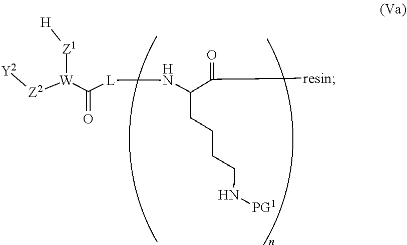

(c) reacting the third resin-bound, side-chain-protected peptide with one or more amino acids corresponding to the amino acids of the polypeptide represented by Y¹ to provide a fourth resin-bound, side-chain protected peptide having a structure according to Formula (VIa):

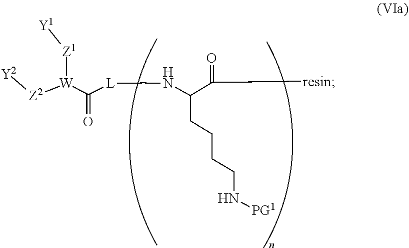

and
(d) cleaving the fourth resin-bound, side-chain-protected peptide from the resin to provide the covalent protein dimer.

In some embodiments, the covalent protein dimer of Formula (I) has a structure according to Formula (Ib):

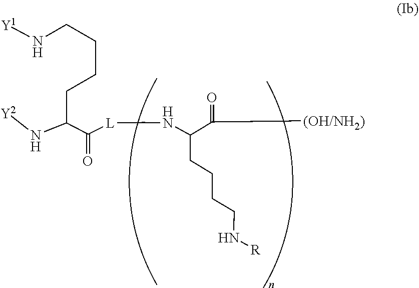

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety;
n is 0 or 1;
the method comprising:
(a) reacting a first resin-bound, side-chain-protected peptide having a structure according to Formula (IIIb):

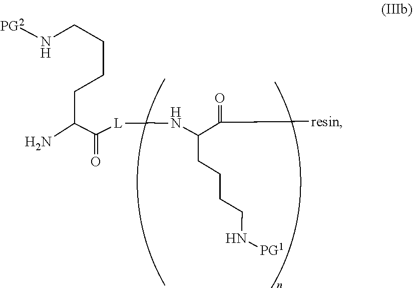

with one or more amino acids corresponding to the amino acids of the polypeptide represented by Y² to provide a second resin-bound, side-chain-protected peptide having a structure according to Formula (IVb):

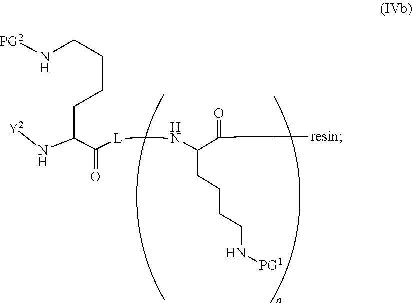

wherein PG¹ and PG² are non-identical nitrogen protecting groups, and wherein neither PG¹ nor PG² are Fmoc;
(b) removing PG² from the second resin-bound, side-chain-protected peptide to provide a third resin-bound, side-chain-protected peptide having a structure according to Formula (Vb):

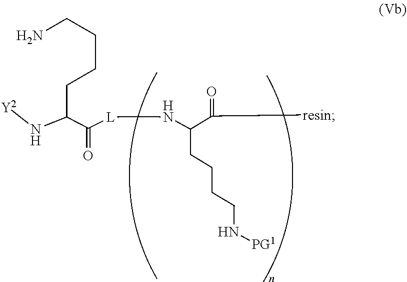

(c) reacting the third resin-bound, side-chain-protected peptide with one or more amino acids corresponding to the amino acids of the polypeptide represented by Y¹ to provide a fourth resin-bound, side-chain protected peptide having a structure according to Formula (VIb):

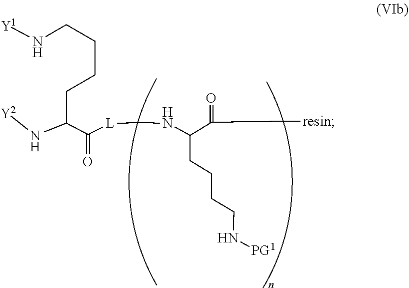

and
(d) cleaving the fourth resin-bound, side-chain-protected peptide from the resin to provide the covalent protein dimer.

In some embodiments, Y¹ and Y² are not identical.

In some embodiments, PG¹ is selected from the group consisting of allyloxycarbonyl (Alloc), carbobenzyloxy (Cbz), tert-butyloxycarbonyl (Boc), acetyl (Ac), benzoyl (Bz), tosyl (Ts), and benzyl (Bn). In some embodiments, PG¹ is Boc.

In some embodiments, Z¹ and Z² are —NH—, and PG² is a nitrogen protecting group. In some embodiments, Z¹ is —NH—, and PG² is selected from the group consisting of allyloxycarbonyl (Alloc), carbobenzyloxy (Cbz), tert-butyloxycarbonyl (Boc), acetyl (Ac), benzoyl (Bz), tosyl (Ts), and benzyl (Bn). In some embodiments, Z¹ is —NH—, and PG² is Alloc.

In some embodiments, prior to step (d), the method comprises removing PG¹ to provide a deprotected nitrogen atom therein, and covalently attaching biotin, a fluorescent dye, a nuclear-targeting moiety, or a cell-penetrating moiety to the deprotected nitrogen atom.

In some embodiments, each of the one or more amino acids of steps (a) and (c) comprises an Fmoc-protected backbone amino group, wherein the corresponding Fmoc group is deprotected after each amino acid is attached to the resin-bound, side-chain-protected peptide.

In some embodiments, each one of steps (a) and (c) is performed in the presence of a coupling agent. Coupling agents suitable for the methods disclosed herein include those known in the art to facilitate peptide bond formation. Exemplary non-limiting coupling agents include (7-azabenzotriazol-1-yloxy)tripyrrolidinophosphonium hexafluorophosphate (PyAOP), hexafluorophosphate azabenzotriazole tetramethyl uronium (HATU), hexafluorophosphate benzotriazole tetramethyl uronium (HBTU), 2-(6-chloro-1H-benzotriazole-1-yl)-1,1,3,3-tetramethylaminium hexafluorophosphate (HCTU), and hydroxybenzotriazole (HOBt).

In some embodiments, each one of steps (a) and (c) comprises the addition of N,N-diisopropylethylamine (DIEA).

In another aspect, the disclosure provides a method of making a covalent protein dimer, or a pharmaceutically acceptable salt thereof, having a structure according to Formula (I):

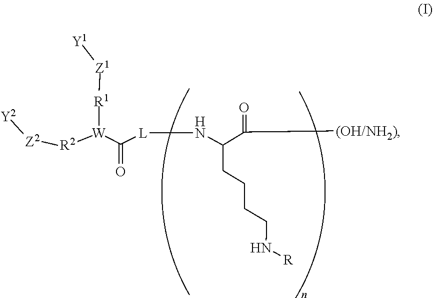

wherein:
Y¹ and Y² are identical and each represents a polypeptide comprising a degree of identity of at least 85% with respect to SEQ ID NO: 1, 2, or 3;
Z¹ is —O—, —NH—, or —S—;
Z² is —O—, —NH—, or —S—;
R¹ is absent, C₁₋₁₀ alkyl, or C₁₋₁₀ heteroalkyl;
R² is absent, C₁₋₁₀ alkyl, or C₁₋₁₀ heteroalkyl;
W is C₁₋₁₀ alkyl, C₁₋₁₀ heteroalkyl, C₆₋₁₀ aryl, or 5- to 10-membered heteroaryl;
L is absent or a linker;
R is H, a protecting group, biotin, a fluorescent dye, a nuclear-targeting moiety, or a cell-penetrating moiety; and
n is 0 or 1;
the method comprising:
(a) reacting a first resin-bound, side-chain-protected peptide having a structure according to Formula (VII):

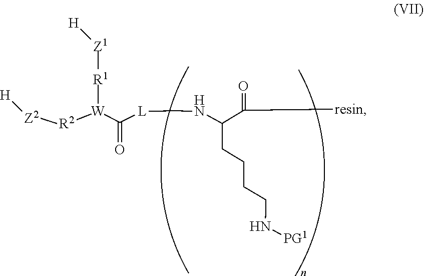

with one or more amino acids corresponding to the amino acids of the polypeptide represented by Y¹ to provide a second resin-bound, side-chain-protected peptide having a structure according to Formula (VI):

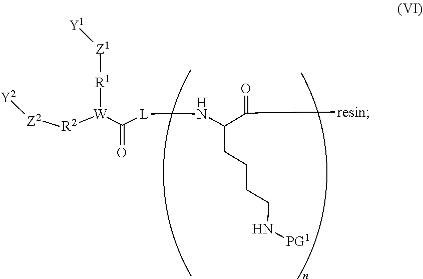

wherein PG¹ is a nitrogen protecting group that is not Fmoc; and
(b) cleaving the second resin-bound, side-chain-protected peptide from the resin to provide the covalent protein dimer.

In some embodiments, the covalent protein dimer of Formula (I) has a structure according to Formula (Ia):

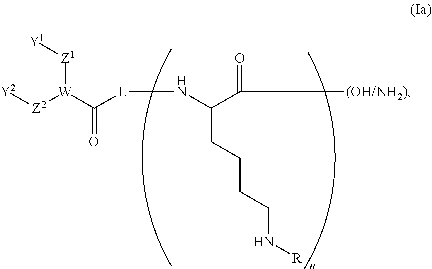

wherein:
Y¹ and Y² are identical and each represents a polypeptide comprising a degree of identity of at least 85% with respect to SEQ ID NO: 1, 2, or 3;
Z¹ is —O—, —NH—, or —S—;
Z² is —O—, —NH—, or —S—;
W is C₁₋₁₀ alkyl or C₁₋₁₀ heteroalkyl;
L is absent or a linker;
R is H, a protecting group, a fluorescent dye, biotin, a nuclear-targeting moiety, or a cell-penetrating moiety; and
n is 0 or 1;
the method comprising:
(a) reacting a first resin-bound, side-chain-protected peptide having a structure according to Formula (VIIa):

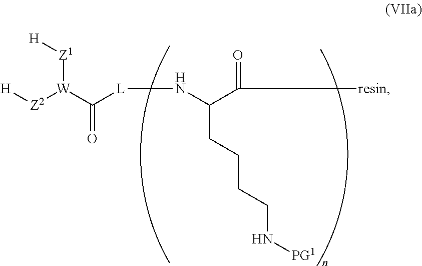

with one or more amino acids corresponding to the amino acids of the polypeptide represented by Y¹ to provide a second resin-bound, side-chain-protected peptide having a structure according to Formula (VIa):

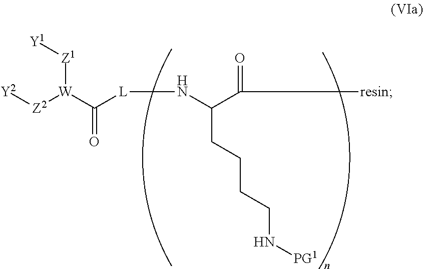

wherein PG¹ is a nitrogen protecting group that is not Fmoc; and
(b) cleaving the second resin-bound, side-chain-protected peptide from the resin to provide the covalent protein dimer.

In some embodiments, the covalent protein dimer of Formula (I) has a structure according to Formula (Ib):

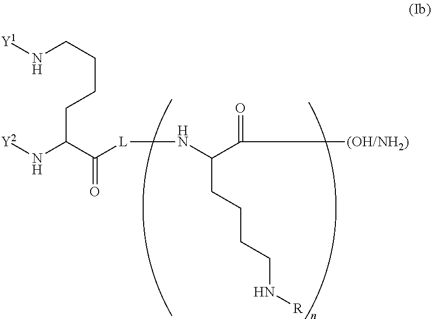

wherein:
Y¹ and Y² are identical and each represents a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety;
n is 0 or 1;
the method comprising:
(a) reacting a first resin-bound, side-chain-protected peptide having a structure according to Formula (VIIb):

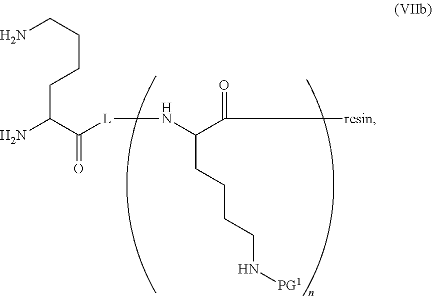

with one or more amino acids corresponding to the amino acids of the polypeptide represented by Y¹ to provide a second resin-bound, side-chain-protected peptide having a structure according to Formula (VIb):

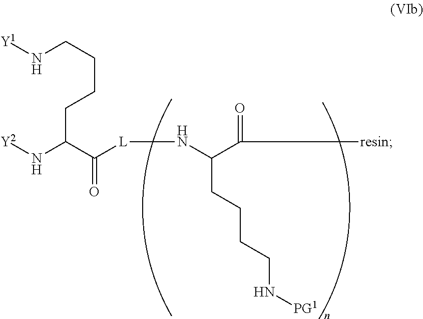

wherein PG¹ is a nitrogen protecting group that is not Fmoc; and
(b) cleaving the second resin-bound, side-chain-protected peptide from the resin to provide the covalent protein dimer.

In some embodiments, PG¹ is selected from the group consisting of allyloxycarbonyl (Alloc), carbobenzyloxy (Cbz), tert-butyloxycarbonyl (Boc), acetyl (Ac), benzoyl (Bz), tosyl (Ts), and benzyl (Bn). In some embodiments, PG¹ is Alloc.

In some embodiments, Z¹ and Z² are —NH—.

In some embodiments, prior to step (b), the method comprises removing PG¹ to provide a deprotected nitrogen atom therein, and covalently attaching biotin, a fluorescent dye, a nuclear-targeting moiety, or a cell-penetrating moiety to the deprotected nitrogen atom.

In some embodiments, each of the one or more amino acids of step (a) comprise an Fmoc-protected backbone amino group, and wherein the corresponding Fmoc group is deprotected after each amino acid is attached to the resin-bound, side-chain-protected peptide.

In some embodiments, step (a) is performed in the presence of a coupling agent. Exemplary non-limiting coupling agents include (7-azabenzotriazol-1-yloxy)tripyrrolidinophosphonium hexafluorophosphate (PyAOP), hexafluorophosphate azabenzotriazole tetramethyl uronium (HATU), hexafluorophosphate benzotriazole tetramethyl uronium (HBTU), 2-(6-chloro-1H-benzotriazole-1-yl)-1,1,3,3-tetramethylaminium hexafluorophosphate (HCTU), and hydroxybenzotriazole (HOBt).

In some embodiments, step (a) comprises the addition of N,N-Diisopropylethylamine (DIEA).

In another aspect, the disclosure provides a method of making a covalent protein dimer, or a pharmaceutically acceptable salt thereof, having a structure according to Formula (II):

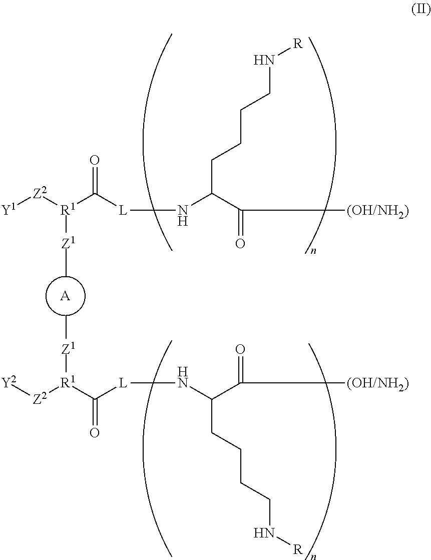

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ independently is —O—, —NH—, or —S—;
Z² independently is —O—, —NH—, or —S—;
R¹ independently is C₁₋₁₀ alkyl or C₁₋₁₀ heteroalkyl;
A is C₆₋₁₀ aryl or 5- to 10-membered heteroaryl;
L independently is absent or a linker;
R independently is H, a nitrogen protecting group, biotin, a fluorescent dye, a nuclear-targeting moiety, or a cell-penetrating moiety; and
n independently is 0 or 1;
the method comprising:
(a) reacting a polypeptide having a structure according to Formula (VIII):

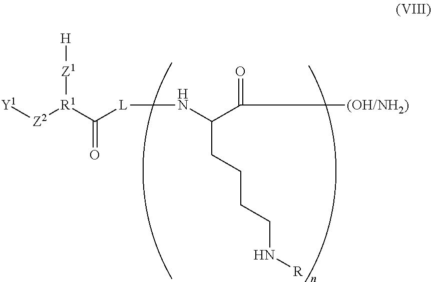

with a compound of Formula (IX):

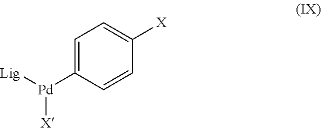

to provide a polypeptide having a structure according to Formula (X):

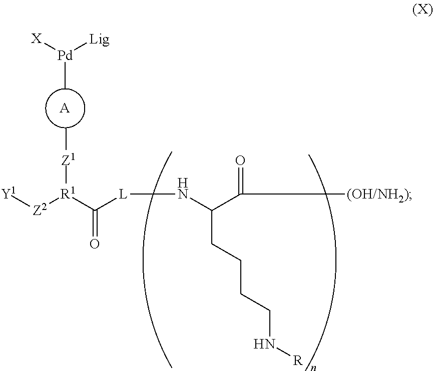

wherein:
X and X′ are each, independently, F, Cl, Br, I, or OTf, and
Lig is a phosphine ligand; and
(b) reacting the polypeptide of Formula (X) with a polypeptide having a structure according to Formula (XI):

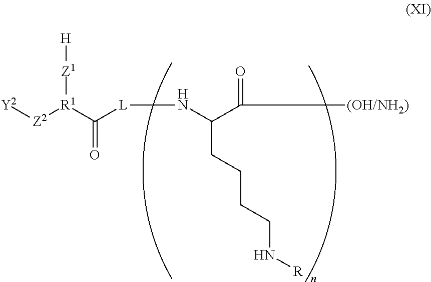

to provide the covalent protein dimer.

In some embodiments, the covalent protein dimer of Formula (II) has a structure according to Formula (IIa):

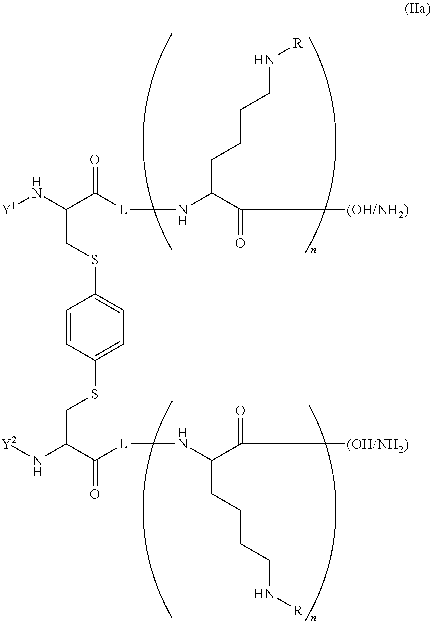

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L independently is absent or a linker,
R independently is H, a nitrogen protecting group, biotin, a fluorescent dye, a nuclear-targeting moiety, or a cell-penetrating moiety; and
n independently is 0 or 1;
the method comprising:
(a) reacting a polypeptide having a structure according to Formula (VIIIa):

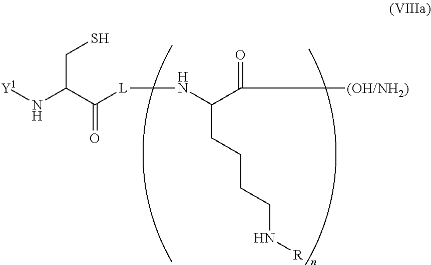

with a compound of Formula (IX):

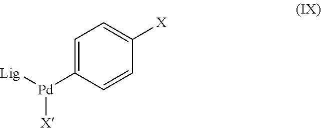

to provide a polypeptide having a structure according to Formula (Xa):

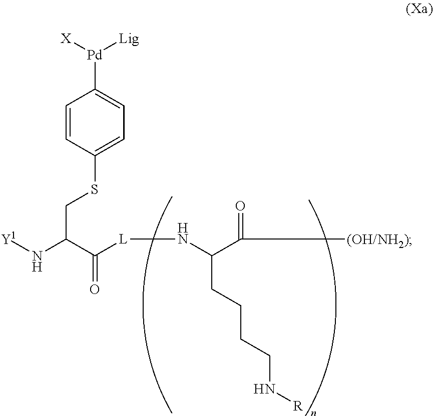

wherein:
X and X′ are each, independently, F, Cl, Br, I, or OTf; and
Lig is a phosphine ligand; and
(b) reacting the polypeptide of Formula (Xa) with a polypeptide having a structure according to Formula (XIa):

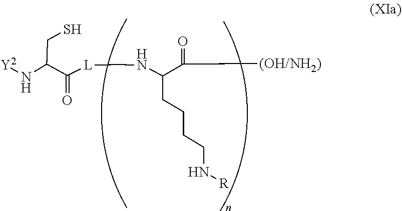

to provide the covalent protein dimer.

In some embodiments, the covalent protein dimer of Formula (II) has a structure according to Formula (IIb):

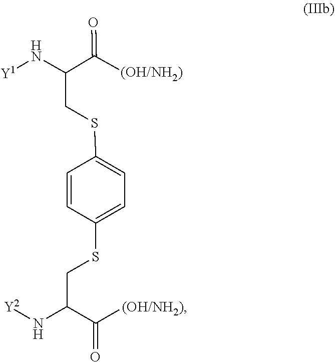

wherein:
Y¹ is a polypeptide comprising a degree of identity of at least 85% with respect to SEQ ID NO: 1, 2, or 3; and
Y² is a polypeptide comprising a degree of identity of at least 85% with respect to SEQ ID NO: 1, 2, or 3;
the method comprising:
(a) reacting a polypeptide having a structure according to Formula (VIIIb):

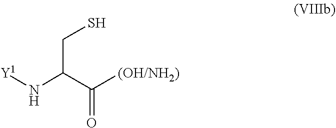

with a compound of Formula (IX):

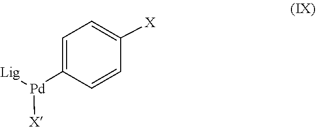

to provide a polypeptide having a structure according to Formula (Xb):

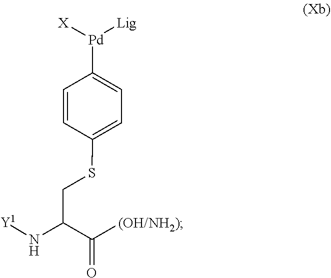

wherein:
X and X′ are each, independently, F, Cl, Br, I, or OTf; and
Lig is a phosphine ligand; and
(b) reacting the polypeptide of Formula (Xb) with a polypeptide having a structure according to Formula (XIb):

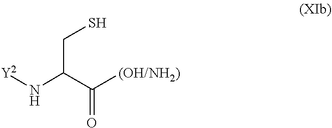

to provide the covalent protein dimer.

In some embodiments, Y¹ and Y² are not identical.

In some embodiments, Z¹ is —S—. In some embodiments, Z² is —NH—.

In some embodiments, the compound of Formula (IX), (IXa), or (IXb) is provided in molar excess with respect to the polypeptide of Formula (VIII), (VIIIa), or (VIIIb). In some embodiments, the compound of Formula (IX), (IXa), or (IXb) and the polypeptide of Formula (VIII), (VIIIa), or (VIIIb) are provided in a molar ratio from about 10:1 to about 2:1. In some embodiments the compound of Formula (IX), (IXa), or (IXb) and the polypeptide of Formula (VIII), (VIIIa), or (VIIIb) are provided in a molar ratio of about 5:1.

In some embodiments, X and X′ are I.
Lig may be any phosphine ligand known in the art to be useful in cross-coupling reactions. By non-limiting example, Lig may be JohnPhos, DavePhos, XPhos, SPhos, MePhos, RuPhos, BrettPhos, PhDavePhos, tBuXPhos, tBuMePhos, tBuBrettPhos, tBuDavePhos, or JackiePhos. In some embodiments, Lig has a structure according to Formula (XII):

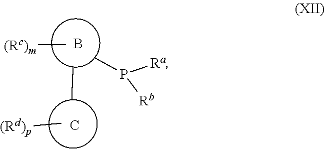

wherein:
B and C are each, independently, C₆₋₁₀ aryl or 6- to 10-membered heteroaryl;
R^{a} and R^{b} are each, independently, C₅₋₁₀ cycloalkyl, C₁₋₆ alkyl, or C₈₋₁₀ aryl, optionally wherein the aryl is substituted with one, two, or three C₁₋₃ haloalkyl groups;
R^{c}, independently is C₁₋₄ alkyl, C₁₋₄ alkoxy, or N (C₁₋₄ alkyl)₂
R^{d}, independently is C₁₋₄ alkyl, C₁₋₄ alkoxy, N(C₁₋₄ alkyl)₂, SO₃H, SO₃M, or C₃₋₁₀ cycloalkyl;
M is Li, Na, or K;
m is 0, 1, 2, 3, or 4; and
p is 1, 2, 3, or 4.

In some embodiments, Lig is

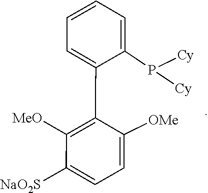

In another aspect, the disclosure provides a method of making a covalent protein dimer having a structure according to Formula (II):

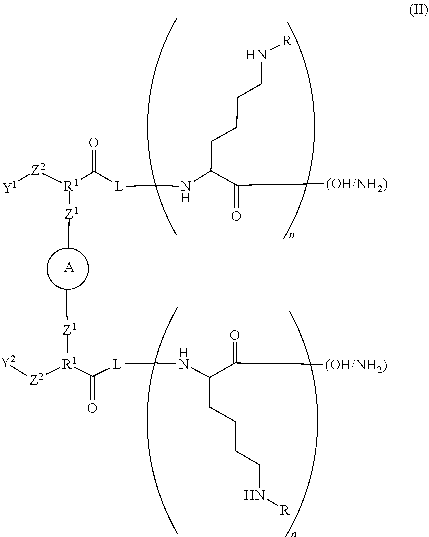

wherein:
Y¹ and Y² are identical and each represents a polypeptide comprising a degree of identity of at least 85% with respect to SEQ ID NO: 1, 2, or 3;
Z¹ independently is —O—, —NH—, or —S—;
Z² independently is —O—, —NH—, or —S—;
R¹ independently is C₁₋₁₀ alkyl or C₁₋₁₀ heteroalkyl;
A is C₆₋₁₀ aryl or 5- to 10-membered heteroaryl;
L independently is absent or a linker;
R independently is H, a nitrogen protecting group, biotin, a fluorescent dye, a nuclear-targeting moiety, or a cell-penetrating moiety; and
n independently is 0 or 1;
the method comprising reacting a polypeptide having a structure according to Formula (VIII):

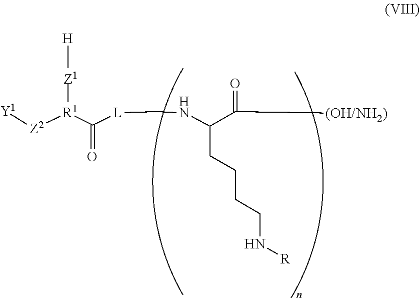

with a compound of Formula (IX):

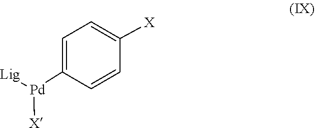

to provide the covalent protein dimer,
wherein:
X and X′ are each, independently, F, Cl, Br, I, or OTf, and
Lig is a phosphine ligand.

In some embodiments, the covalent protein dimer of Formula (II) has a structure according to Formula (IIa):

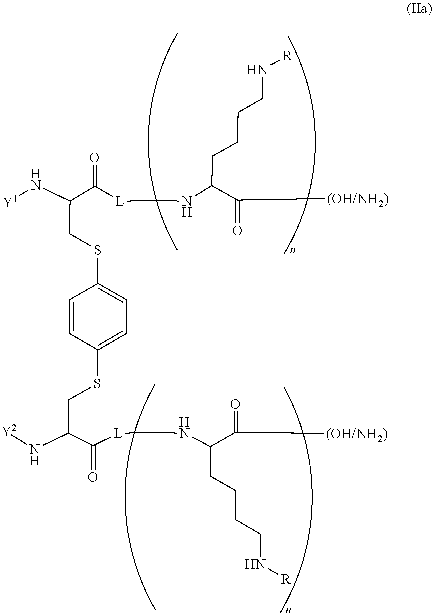

wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L independently is absent or a linker;
R independently is H, a nitrogen protecting group, biotin, a fluorescent dye, a nuclear-targeting moiety, or a cell-penetrating moiety; and
n independently is 0 or 1;
the method comprising reacting a polypeptide having a structure according to Formula (VIIIa):

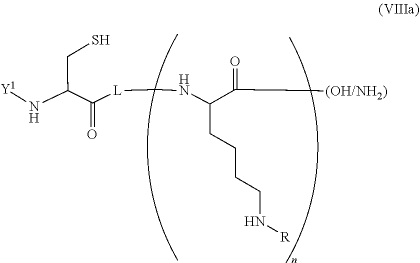

with a compound of Formula (IX):

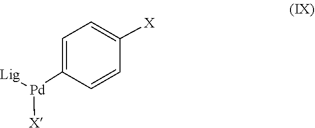

to provide the covalent protein dimer;
wherein:
X and X′ are each, independently, F, Cl, Br, I, or OTf; and
Lig is a phosphine ligand.

In some embodiments, the covalent protein dimer of Formula (II) has a structure according to Formula (IIb):

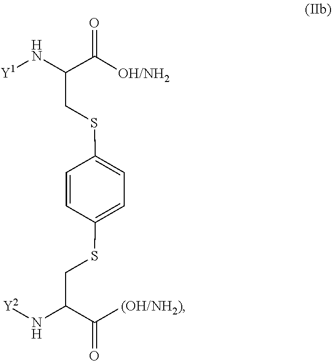

wherein:
Y¹ and Y² are identical and each represents a polypeptide comprising a degree of identity of at least 85% with respect to SEQ ID NO: 1, 2, or 3;
the method comprising reacting a polypeptide having a structure according to Formula (VIIIb):

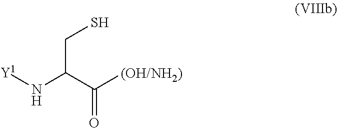

with a compound of Formula (IX):

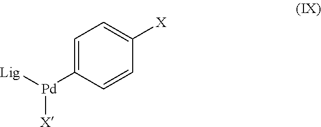

to provide the covalent protein dimer,
wherein:
X and X′ are each, independently, F, Cl, Br, I, or OTf; and
Lig is a phosphine ligand.
In some embodiments, Z¹ is —S—. In some embodiments, Z² is —NH—.
In some embodiments, X and X′ are I.
Lig may be any phosphine ligand known in the art to be useful in cross-coupling reactions. By non-limiting example, Lig may be JohnPhos, DavePhos, XPhos, SPhos, MePhos, RuPhos, BrettPhos, PhDavePhos, tBuXPhos, tBuMePhos, tBuBrettPhos, tBuDavePhos, or JackiePhos. In some embodiments, Lig has a structure according to Formula (XII):

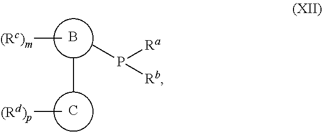

wherein:
B and C are each, independently, C₆₋₁₀ aryl or 6- to 10-membered heteroaryl;
R^{a} and R^{b} are each, independently, C₅₋₁₀ cycloalkyl, C₁₋₆ alkyl, or C₈₋₁₀ aryl, optionally wherein the aryl is substituted with one, two, or three C₁₋₃ haloalkyl groups;
R^{c}, independently is C₁₋₄ alkyl, C₁₋₄ alkoxy, or N(C₁₋₄ alkyl)₂
R^{d}, independently is C₁₋₄ alkyl, C₁₋₄ alkoxy, N(C₁₋₄ alkyl)₂, SO₃H, SO₃M, or C₃₋₁₀ cycloalkyl;
M is Li, Na, or K;
m is 0, 1, 2, 3, or 4; and
p is 1, 2, 3, or 4.

In some embodiments, Lig is

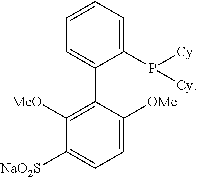

### Methods of Treatment

In an aspect, provided herein is a method of treating a disease or disorder characterized by MYC dysregulation in a subject in need thereof, the method comprising administering to the subject a covalent protein dimer of the present disclosure. In some embodiments, the disease or disorder characterized by MYC dysregulation is an immune disorder, such as myasthenia gravis, psoriasis, pemphigus vulgaris, and atherosclerosis. In some embodiments, the disease or disorder is cancer. In certain embodiments, the cancer is selected from the group consisting of pancreatic cancer, lung cancer, prostate cancer, breast cancer, ovarian cancer, kidney cancer, liver cancer, brain cancer, neuroblastoma, colorectal cancer, and hematological malignancies.

Also described are methods for contacting a cell or a biological sample with an effective amount of a covalent protein dimer of the disclosure.

In yet another aspect, provided herein is a method of treating cancer in a subject in need thereof, the method comprising administering to the subject a covalent protein dimer of the present disclosure.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the covalent protein dimers described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In some embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, ovarian cancer, glioma, squamous cell carcinoma, or prostate cancer. In some embodiments, the cancer is breast cancer, colorectal cancer, pancreatic cancer, gastric cancer, or uterine cancer. In some embodiments, the cancer is a hematological malignancy. In some embodiments, the cancer is acute myeloid leukemia, chronic myelogenous leukemia, Hodgkin's lymphoma, or diffuse large B-cell lymphoma. In some embodiments, the cancer is lung cancer. In some embodiments, the cancer is a non-small cell lung cancer.

In some embodiments, the covalent protein dimes of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the covalent protein dimers of this disclosure are useful for treating hematopoietic disorders acute-myelogenous leukemia (AML), chronic-myelogenous leukemia (CML), acute-promyelocytic leukemia, and acute lymphocytic leukemia (ALL).

In one aspect, the present disclosure provides for the use of one or more covalent protein dimers of the disclosure in the manufacture of a medicament for the treatment of cancer, including without limitation the various types of cancer disclosed herein.

### Formulations and Dosages

The covalent protein dimers described herein will generally be administered to a subject as a pharmaceutical composition. The terms “patient” and “subject”, as used herein, include humans and non-human animals. The covalent protein dimers described herein may be employed therapeutically, under the guidance of a physician.

The compositions comprising the covalent protein dimers of the instant disclosure may be conveniently formulated for administration with any pharmaceutically acceptable carrier(s). For example, the covalent protein dimers may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the covalent protein dimers in the chosen medium may be varied and the medium may be chosen based on the desired route of administration of the pharmaceutical composition. Except insofar as any conventional media or agent is incompatible with the covalent protein dimers to be administered, its use in the pharmaceutical composition is contemplated.

The dose and dosage regimen of the covalent protein dimers disclosed herein that are suitable for administration to a particular subject may be determined by a physician considering the subject's age, sex, weight, general medical condition, and the specific condition for which the covalent protein dimer(s) is being administered and the severity thereof. The physician may also take into account the route of administration, the pharmaceutical carrier, and the covalent protein dimers' biological activity.

Selection of a suitable pharmaceutical composition will also depend upon the mode of administration chosen. For example, the covalent protein dimers of the invention may be administered by direct injection to a desired site (e.g., tumor). In this instance, a pharmaceutical composition comprising the covalent protein dimers is dispersed in a medium that is compatible with the site of injection. Covalent protein dimers of the instant disclosure may be administered by any method. For example, the covalent protein dimers of the instant disclosure can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly.

Pharmaceutical compositions containing a covalent protein dimer of the present disclosure as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical composition of the disclosure may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the subject. Appropriate concentrations for alleviation of a particular pathological condition may be determined by dosage concentration curve calculations, as known in the art.

In accordance with the present disclosure, the appropriate dosage unit for the administration of covalent protein dimers may be determined by evaluating the toxicity of the molecules or cells in animal models. Various concentrations of covalent protein dimers in pharmaceutical preparations may be administered to mice, and the minimal and maximal dosages may be determined based on the beneficial results and side effects observed as a result of the treatment. Appropriate dosage units may also be determined by assessing the efficacy of the covalent protein dimers in combination with other standard drugs. The dosage units of covalent protein dimers may be determined individually or in combination with each treatment according to the effect detected.

The pharmaceutical compositions comprising the covalent protein dimers may be administered at appropriate intervals, for example, at least twice a day or more until the pathological symptoms are reduced or alleviated, after which the dosage may be reduced to a maintenance level. The appropriate interval in a particular case would normally depend on the condition of the subject.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Synthesis of Lysine-Linked Covalent Protein Dimers

### Manual Preparation of Peptidyl Resins 1 and 2

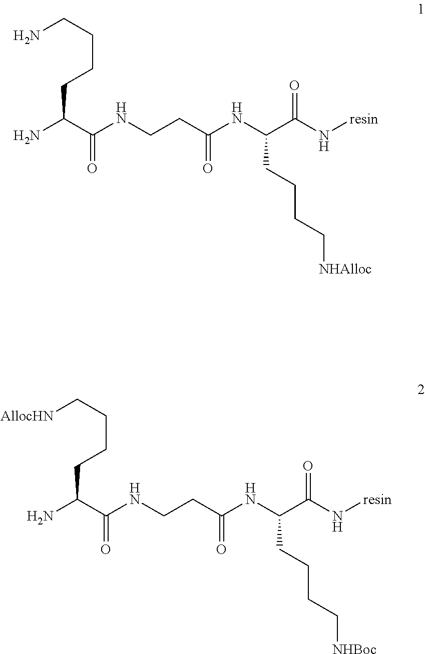

Preparation of ChemMatrix® Rink amide resin (loading 0.18 mmol/g, typical scale: 100 mg, 0.02 mmol) was loaded into a fritted syringe (6 mL), swollen in DMF (4 mL) for 5 minutes and then drained. Each Na-Fmoc protected amino acid (0.2 mmol, 10 equiv.) was dissolved in DMF containing 0.39 M HATU (0.5 mL). Immediately before the coupling, DIEA (100 μL, 30 equiv.) was added to the mixture to activate the amino acid. After 15 seconds preactivation, the mixture was added to the resin and reacted for 10 min, with occasional stirring. After completion of the coupling step, the syringe was drained, and the resin was washed with DMF (3×5 mL). Fmoc deprotection was performed by addition of piperidine (20% in DMF, 3 mL) to the resin (1×1 min+1×5 min), followed by draining and washing the resin with DMF (5×5 mL). For peptidyl resin 1 the coupling cycles were performed sequentially with Fmoc-Lys (Alloc)—OH, Fmoc-βAla-OH, and Fmoc-Lys (Fmoc)—OH; for peptidyl resin 2 the coupling cycles were performed sequentially with Fmoc-Lys (Boc)—OH, Fmoc-βAla-OH and Fmoc-Lys (Alloc)—OH.

### Automated Flow Peptide Synthesis (AFPS)

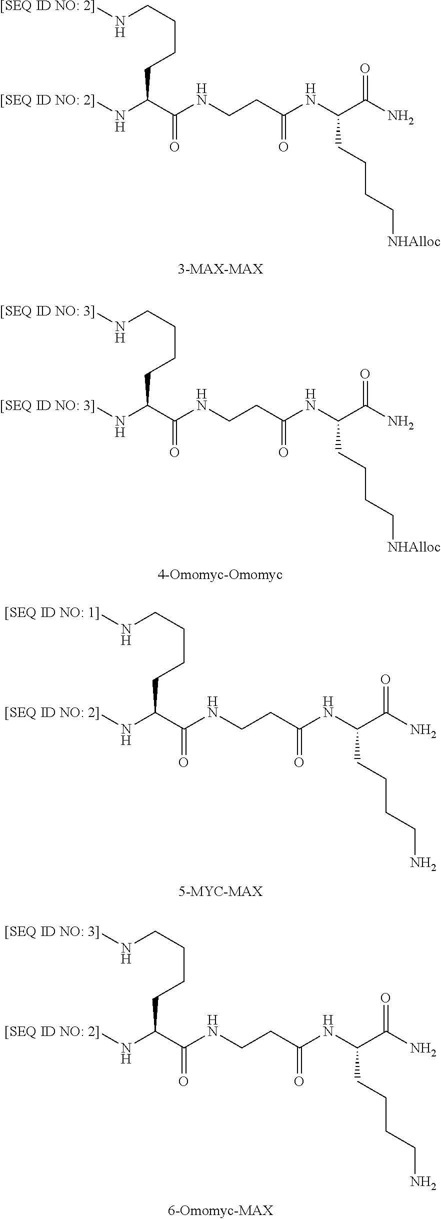

Covalent MAX-MAX and Omomyc-Omomyc homodimers were prepared via parallel single-shot fast-flow solid-phase synthesis from peptidyl resin 1. Each step involved the parallel coupling and subsequent deprotection of two amino acids simultaneously. The synthesis time for each homodimer was about 3.5 hours (MAX-MAX (3), 164 residues; Omomyc-Omomyc (4), 184 residues). After cleavage and side-chain deprotection, LC-MS analysis indicated the desired products as the major component of both crude reaction mixtures. Upon preparative HPLC purification, pure MAX-MAX (3) and Omomyc-Omomyc (4) were obtained in 6% and 8% yield, respectively.

Covalent MYC-MAX and Omomyc-MAX heterodimers were prepared by consecutive single-shot fast flow solid phase synthesis. With the fast flow synthesizer, MAX was assembled from the α-amine of the lysine linker of peptidyl resin 2. For the last amino acid, Boc-glycine was added, and the Alloc protection was removed from the NE of the lysine linker. On this amine, MYC or Omomyc were assembled to provide 5 and 6, respectively. The synthesis time for each heterodimer amounted to about 8 hours (MYC-MAX (5), 167 residues; Omomyc-MAX (6), 175 residues). Both heterodimers were observed as the main component of the crude product mixture obtained from cleavage and side-chain deprotection. Upon preparative HPLC purification, pure MYC-MAX (5) and Omomyc-MAX (6) dimers were obtained in 4% and 5% yield, respectively.

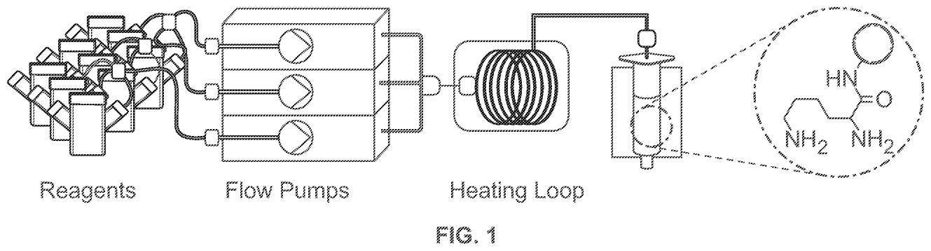

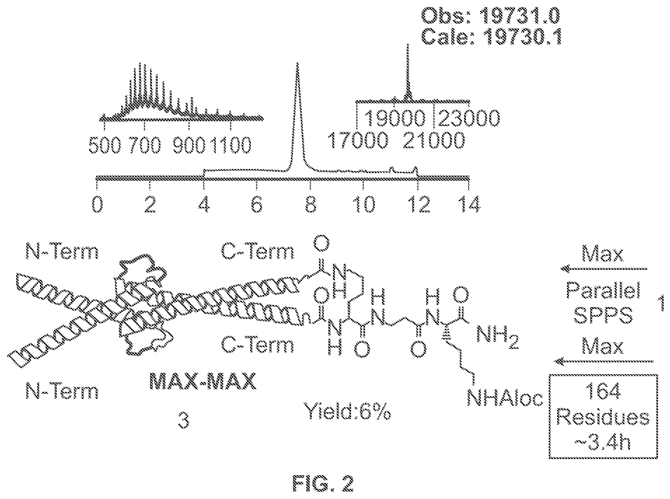

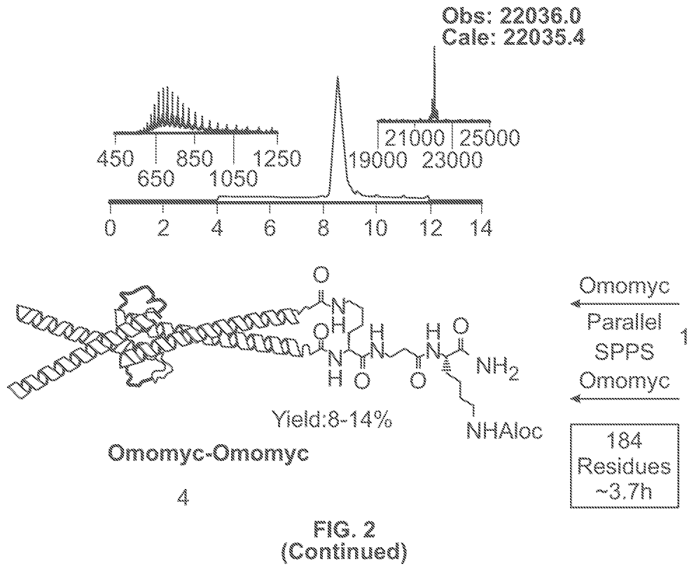

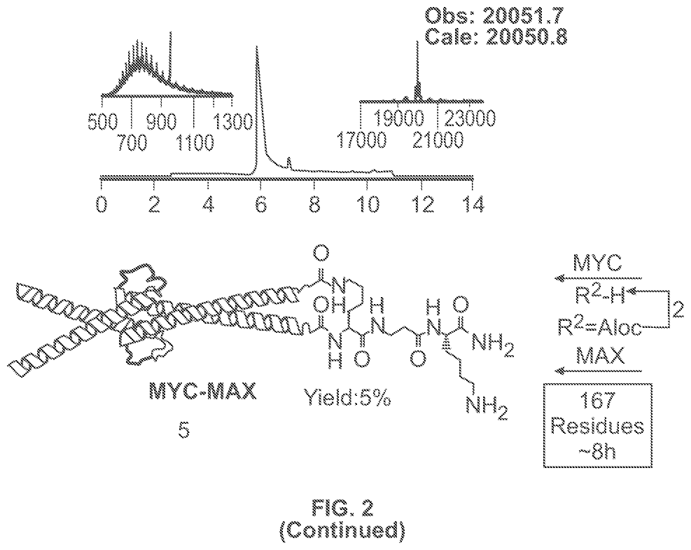

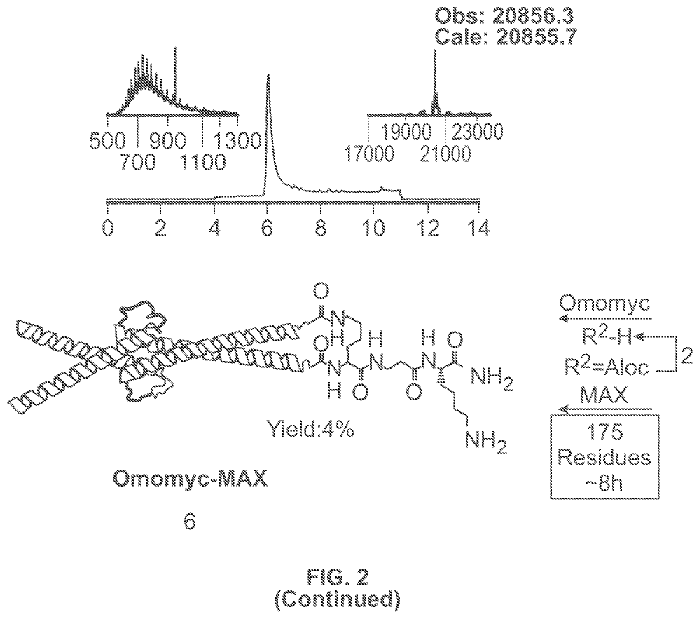

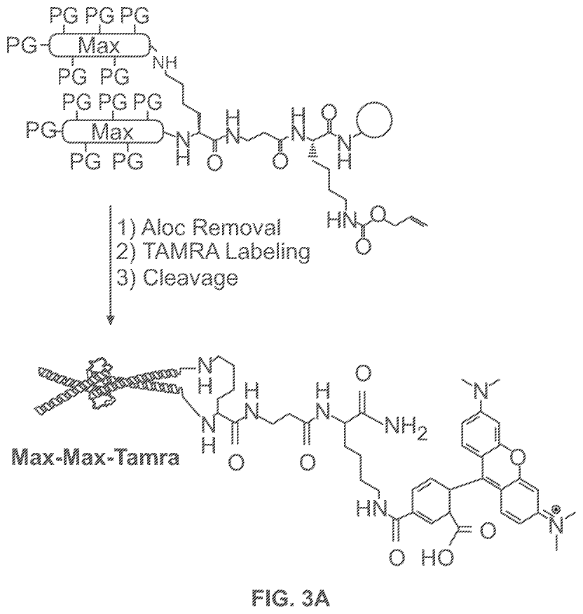

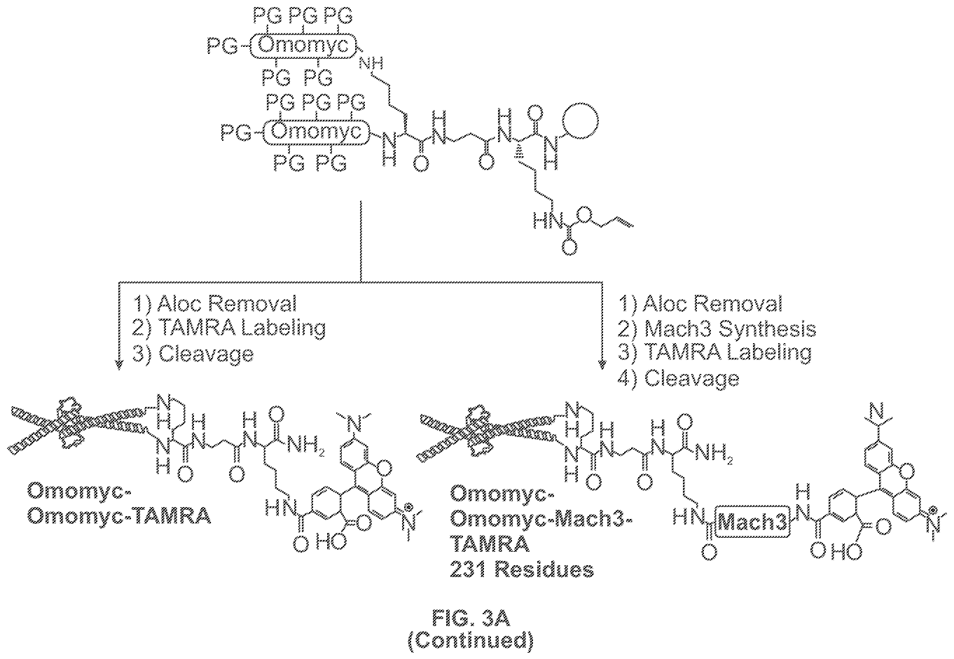

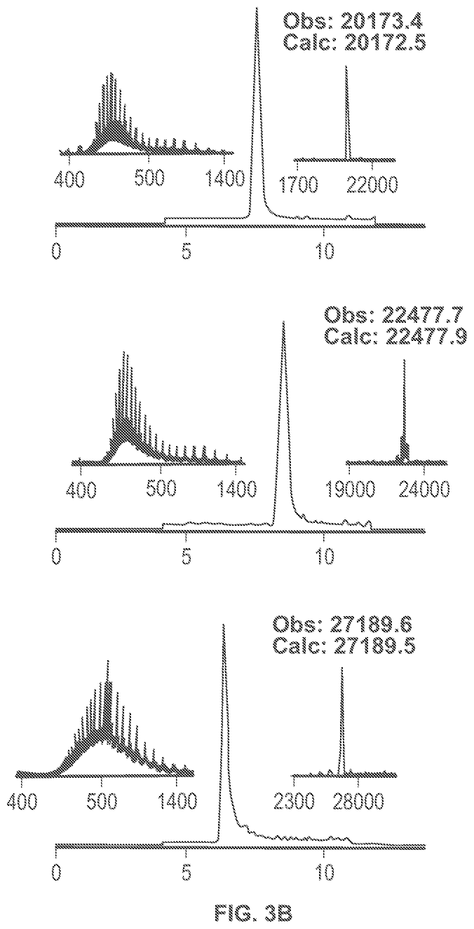

All peptides were synthesized on two automated-flow systems depicted in FIG. 1 and as described in Mijalis, A. J. et al. Nat. Chem. Biol. 13, 464-466 (2017). The synthesis conditions used are according to Hartrampf, N. et al. Science 368, 980-987 (2020). Flow-rate=40 mL/min, temperature =90° C. (loop 1), 70° C. (loop 2; used for histidine) and 85-90° C. (reactor). The 50 ml/min pump head pumps 400 μL of liquid per pump stroke; the 5 mL/min pump head pumps 40 μL of liquid per pump stroke. The standard synthetic cycle involved a first step of prewashing the resin at elevated temperatures for 60 s at 40 mL/min. During the coupling step, three HPLC pumps were used: a 50 mL/min pump head pumped the activating agent, a second 50 ml/min pump head pumped the amino acid, and a 5 mL/min pump head pumped DIEA. The first two pumps were activated for 8 pumping strokes in order to prime the coupling agent and amino acid before the DIEA pump was activated. The three pumps were then actuated together for a period of 7 pumping strokes, after which the activating agent pump and amino acid pump were switched using a rotary valve to select DMF. The three pumps were actuated together for a final 8 pumping strokes, after which the DIEA pump was shut off and the other two pumps continued to wash the resin for another 40 pump strokes. During the deprotection step, two HPLC pumps were used. Using a rotary valve, one HPLC pump selected deprotection stock solution and DMF. The pumps were activated for 13 pump strokes. Both solutions were mixed in a 1:1 ratio. Next, the rotary valves selected DMF for both HPLC pumps, and the resin was washed for an additional 40 pump strokes. The coupling-deprotection cycle was repeated for all additional monomers.

### Manual Boc-Gly-OH Coupling

For heterodimers 5 and 6, prior to site-selective modification via the Alloc protected lysine, the protein N-termini was blocked with Boc-Gly-OH: Peptidyl resin (˜10 μmol theoretical loading) was loaded into a fritted syringe (6 mL), swollen in DMF (4 mL) for 5 minutes and then drained. Boc-Gly-OH (18 mg, 100 μmol) and HATU (54 mg, 90 μmol) were dissolved in DMF (250 μL), activated with DIEA (38 mg, 52 μL, 300 μmol), added to the peptidyl resin and incubated for 15 minutes. After this time, the resin was drained, washed with DMF (3×5 mL) and used for the next step.

### Alloc Deprotection

The peptidyl resin (˜10 μmol theoretical loading) was washed with dichloromethane (3×5 mL) and then treated with Pd(PPh₃)₄ (11.0 mg, 10 μmol, 1 equiv) in dichloromethane/piperidine (8:2, 1 mL) for 30 minutes at room temperature under exclusion of light. The resin was then drained and washed with dichloromethane (3×5 mL).

### Mach3 and TAMRA Conjugation

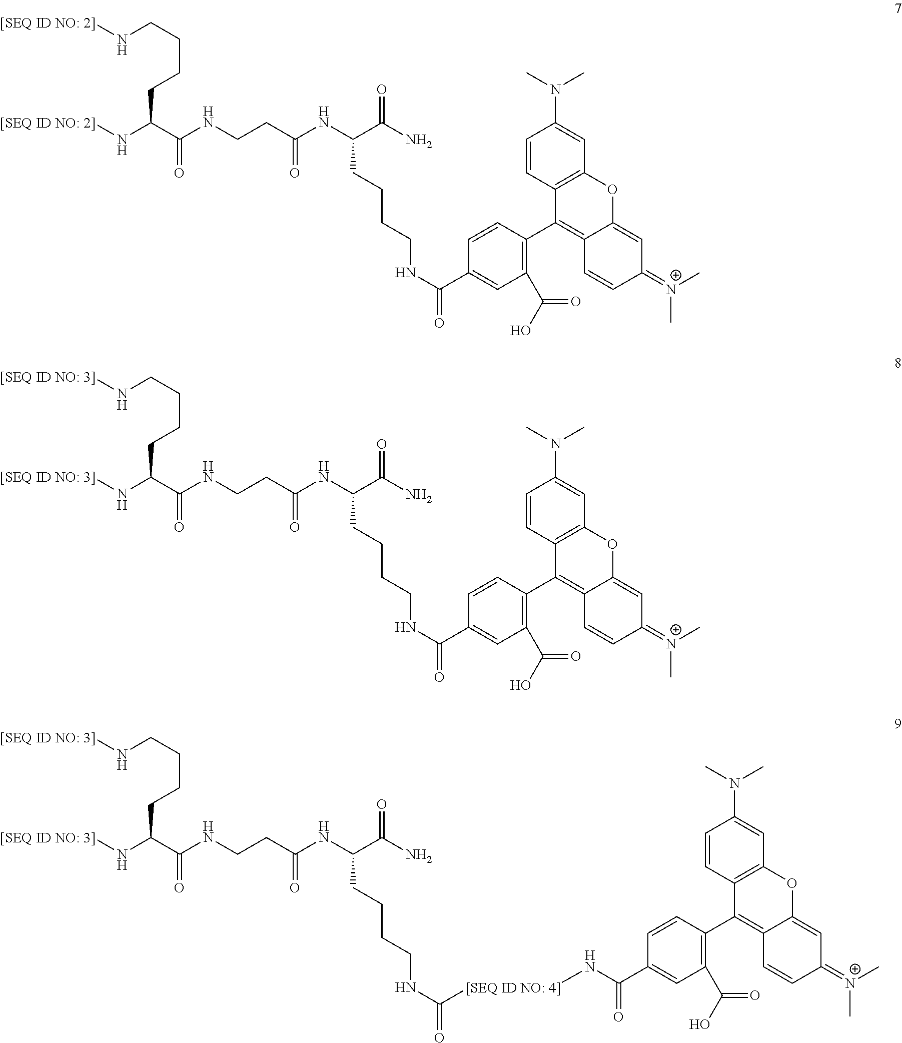

Starting from homodimer 3 or 4, N-termini Boc-protection and Alloc deprotection of the C-terminal lysine was performed according to the protocols above. Mach3 (SEQ ID NO: 4) was installed via AFPS from the resulting free amine. For TAMRA installation, the peptidyl resin (˜10 μmol theoretical loading) was loaded into a fritted syringe (6 mL), swollen in DMF (4 mL) for 5 minutes and then drained. 5-Carboxytetramethylrhodamine (5-TAMRA, 22 mg, 50 μmol, 5 equivalents) and HATU (17 mg, 45 μmol, 4.5 equivalents) were dissolved in DMF (500 μL), activated with DIEA (19 mg, 26 μL, 150 μmol), added to the peptidyl resin and incubated for 30 minutes under exclusion of light. After this time, the resin was drained, washed with DMF (3×5 mL), and stored until cleavage.

### Cleavage Protocol

After synthesis, the peptidyl resin was washed with dichloromethane (3×5 mL) and dried. Approximately 8 mL of cleavage solution (82.5% TFA, 5% water, 5% phenol, 5% thioanisole, 2.5% EDT) was added to the peptidyl resin inside the fritted syringe. The cleavage was kept at room temperature for 4 h, with occasional shaking. After this time, the cleavage mixture was transferred to a falcon tube (through the syringe frit, keeping the resin in the syringe), and the resin washed with an additional 2 mL of cleavage solution. Ice cold diethyl ether (45 mL) was added to the cleavage mixture and the precipitate was collected by centrifugation and triturated twice more with cold diethyl ether (45 mL). The supernatant was discarded. Residual ether was allowed to evaporate, and the peptide was dissolved in 50% acetonitrile in water with 0.1% TFA (long peptides were dissolved 70% acetonitrile in water with 0.1% TFA). The peptide solution was filtrated with a Nylon 0.22 um syringe filter and frozen and then lyophilized until dry.

### Example 2

### Characterization of Lysine-Linked Covalent Protein Dimers

Biophysical characterization confirmed the folding and DNA-binding activity of the four covalent protein dimers 3, 4, 5, and 6. The dimers were first analyzed by sodium dodecyl sulfate polyacrylamide gel electrophoresis (SDS-PAGE). All dimer constructs had bands at the expected height of ˜20 kDa, and the monomers MYC, MAX, and Omomyc (synthesized by AFPS) were observed at ˜10 kDa. The refolding of the protein dimers did not require special procedures. The lyophilized dimers were dissolved in folding buffer (MES 10 mM, KCl 150 mM, MgCl₂ 1 mM, TCEP 1 mM, glycerol 10%, pH=6.5) and all four dimers displayed defined α-helical signatures, as determined by circular dichroism (CD). Next an electrophoretic mobility shift assay (EMSA) was performed to determine the dimers' DNA binding activity. At a DNA concentration of 1 μM and protein concentration of 2 μM, all dimers formed complexes with the E-Box DNA, as observed by the shift retardation on the gel. Monomeric MYC, tested as a negative control (4 μM), did not bind to E-box DNA.

Covalently linked dimers have stabilized structures in aqueous buffer compared to their non-covalent analogs. CD signals at 221 nm between 4 and 89° C. were recorded to determine melting temperatures (Tm). Using this method, the non-covalent MAX/MAX and Omomyc/Omomyc dimers were compared to the four synthetic covalent protein dimers (3, 4, 5, and 6). Protein melting temperature measurements were also performed in the presence of equimolar E-Box DNA. Overall the DNA stabilized the protein complexes' structures. The covalent linkage showed a significant stabilizing effect on the MAX dimers: The Tm of the non-covalent MAX structure was determined to be 29° C. while the Tm of the covalent dimer was 38° C. Omomyc complexes, overall, displayed higher structural stability than the other dimers tested. A significant Tm difference was not observed for non-covalent Omomyc compared to covalent Omomyc-Omomyc (4). This observation might be explained by the greater stability of the Omomyc leucine zipper. The most stable complex of all structures tested was the covalent Omomyc-Omomyc dimer (4) in the presence of DNA, with a Tm of 67° C. Finally, the proteolytic stability of dimer 4 was tested. After 1 h incubation in human serum (5% in PBS) at 37° C. 91% of intact protein dimer was found.

### Polyacrylamide Gel Electrophoresis (PAGE)

SDS-PAGE analysis was performed using Bolt™ 4-12% Bis-Tris Plus Gels (10-wells) at 165 V for 36 min utilizing pre-stained Invitrogen SeeBlue™ Plus2 molecular weight standard. Bolt™ LDS Sample Buffer (4×) was added to each protein sample (1 μg) for loading on the gel. The bands were visualized by Coomassie blue staining.

### Electrophoretic Mobility Shift Assay (EMSA)

The E-Box DNA probe (2 μM in binding buffer) was heated to 95° C. for 5 minutes and then let cool down to room temperature over 15 minutes for double-strand annealing. Protein dimer (4 μM in binding buffer: MES 10 mM, KCl 150 mM, MgCl₂ 1 mM, TCEP 1 mM, glycerol 10%, pH=6.5) was added to the DNA (final concentrations: 2 μM protein and 1 μM DNA) and the mixture was incubated for 1 h at room temperature. During the incubation, a 10% polyacrylamide gel was prerun (1 h, 4° C., 100 V) in 1× TBE buffer. After that time, DNA protein mixture (20 μL) was mixed with 6× DNA Loading Dye (4 μL)) and loaded on the gel, which was run at 75 V, for 90 min at 4° C. The gel was washed with water for 20 seconds and then stained with 0.02% ethidium bromide in 1xTBE buffer for 15 min at room temperature. Bands were visualized on a Biorad Gel imager.

### Circular Dichroism (CD)

Lyophilized samples were dissolved in folding buffer (MES 10 mM, KCl 150 mM, MgCl₂ 1 mM, TCEP 1 mM, glycerol 10%, pH=6.5) at a final protein concentration of 0.1 mg/mL. The circular dichroism (CD) spectra were obtained using an AVIV 420 circular dichroism spectrometer with a 1 mm path length quartz cuvette. 300 μL sample were used for each measurement. For full wavelength scans the CD spectra were recorded from 250 to 200 nm at 4° C. with three seconds averaging times at each wavelength. Y-axis values are reported in molar ellipticity. For melting temperature determination CD spectra were recorded at 221 nm from 4 to 89° C., with +5 degree steps and equilibration times of 60 seconds at each temperature. For measurements in of DNA/protein complexes equimolar E-Box DNA was added to the proteins in folding buffer; the mixtures were heated to 95° C. for 5 minutes, let cool down to room temperature over 15 minutes and then analyzed.

### Example 3

### Cell Penetration of Covalent Protein Dimers

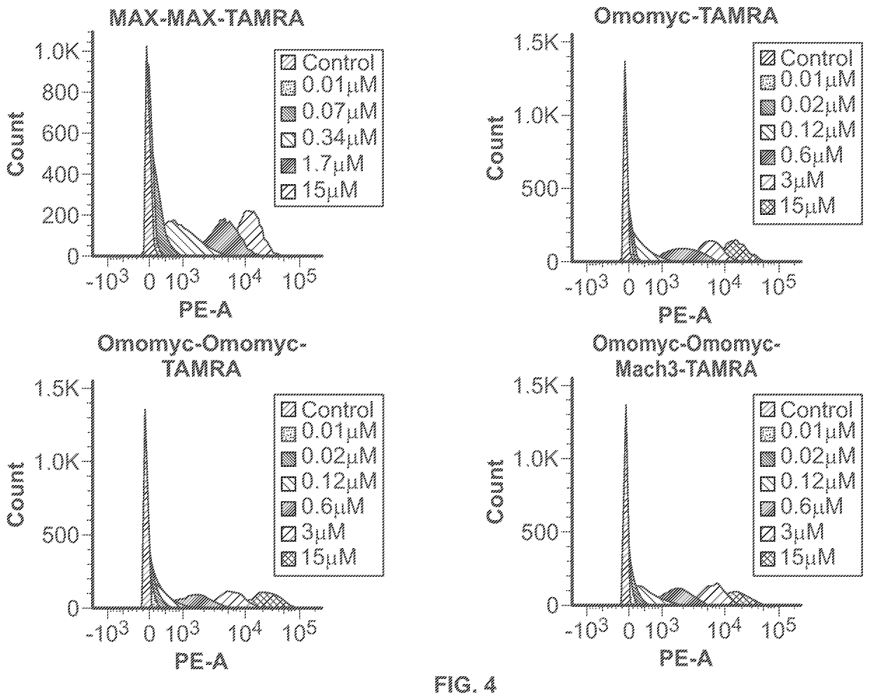

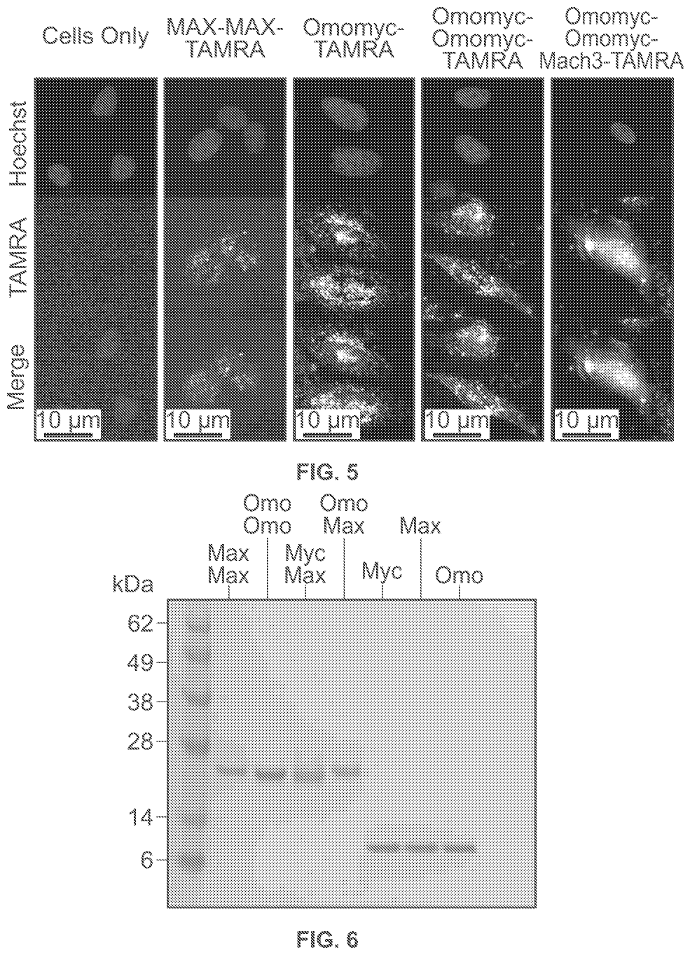

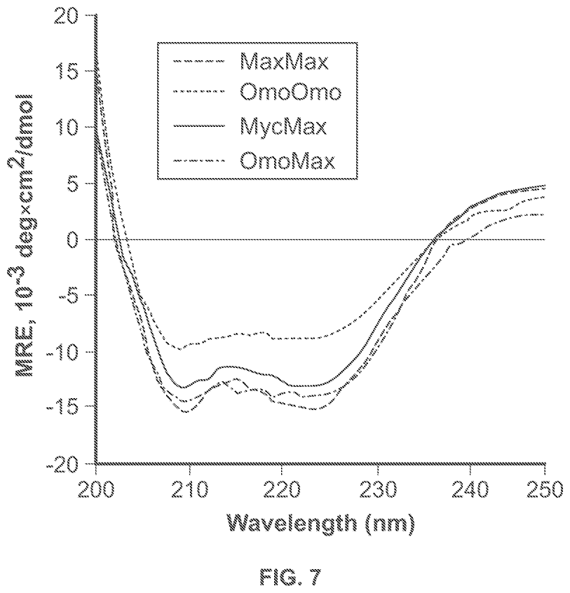

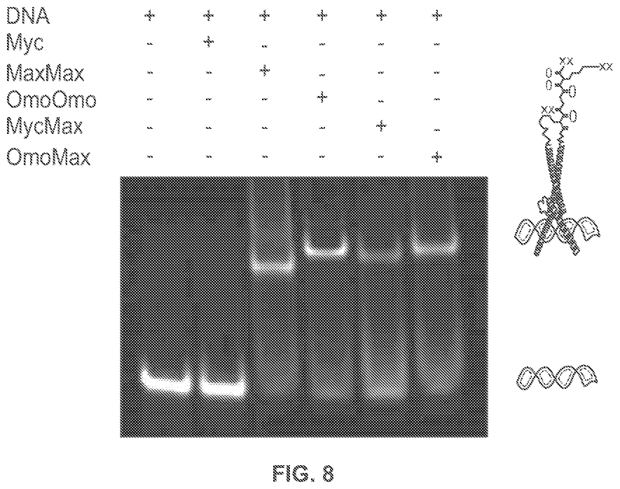

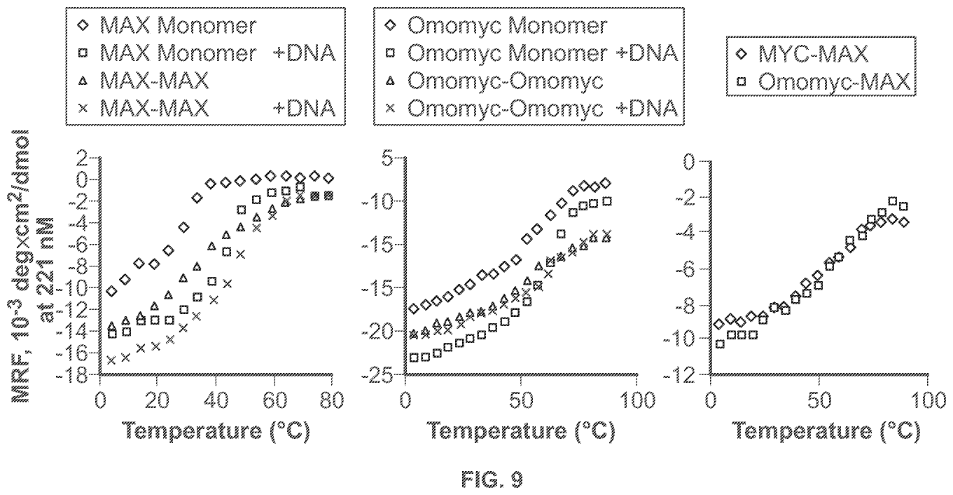

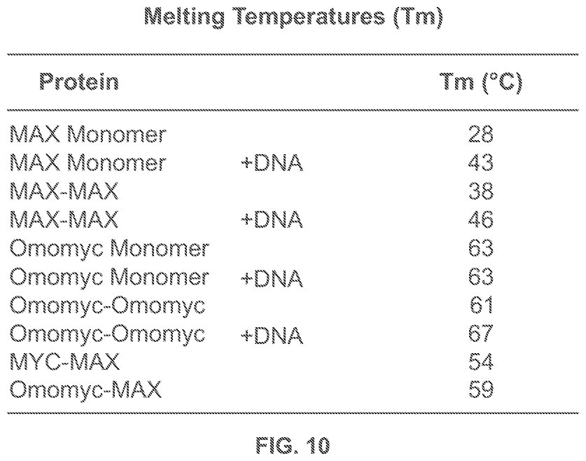

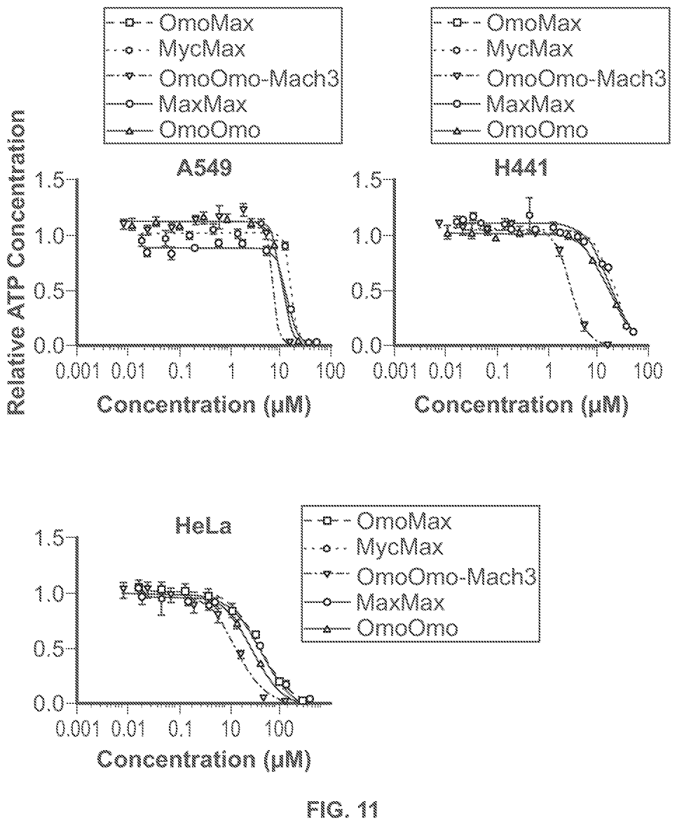

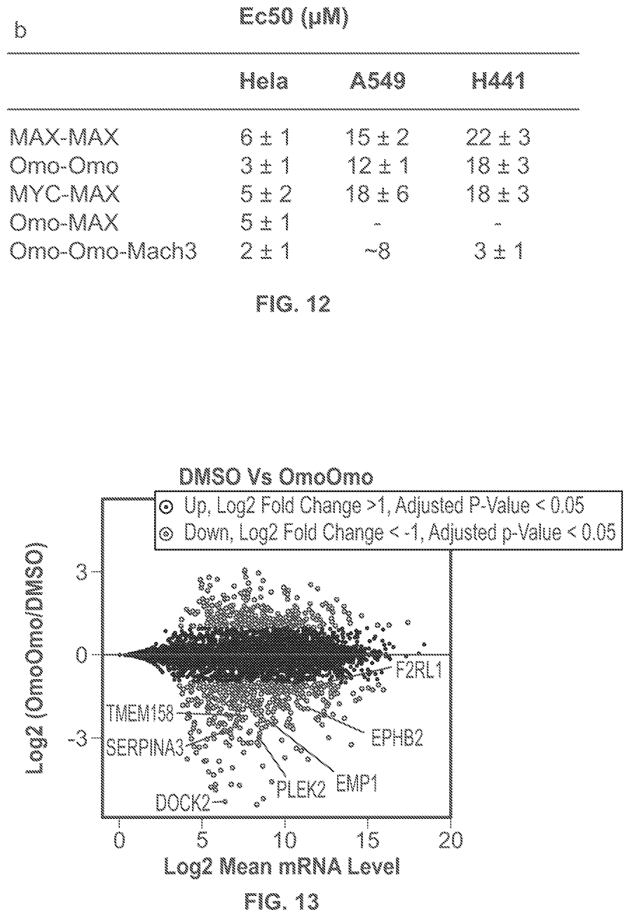

Covalent dimers 7, 8, and 9 were used to assess cell penetration via microscopy and flow cytometry. To evaluate uptake, Hela cells were treated with fluorophore-labeled dimers (7, 8, and 9) and fluorescence was measured via flow cytometry. All three analogs are taken up into cells in a dose-dependent manner after a brief (15 min) incubation (FIG. 4). Addition of (4′,6-Diamidin-2-phenylindol) DAPI as a membrane-impermeable viability dye showed no staining of the gated population of TAMRA-fluorescent cells, suggesting that the constructs entered cells without compromising the membrane. These findings were confirmed by fluorescent microscopy. Treatment of Hela cells for 15 min with the covalent dimers followed by 1 h incubation in fresh media and imaging via confocal microscopy revealed intense, punctate fluorescence, in agreement with previous observations for monomeric Omomyc (FIG. 5). However, treatment with 9 resulted in punctate fluorescence as well as diffuse fluorescence in the nucleus, indicating endosomal escape and nuclear localization (FIG. 5). These experiments show that the dimeric transcription factors are rapidly taken up into cells, and their nuclear localization can be improved with the addition of a non-natural targeting sequence.

### Cell Culture

HeLa (ATCC CCL-2), A549 (ATCC CCL-185), and H441 (ATCC HTB-174) cancer cell lines were maintained in MEM, FK-12, and RPMI-1640 media each containing 10% v/v fetal bovine serum (FBS) and 1% v/v penicillin-streptomycin, respectively, at 37° C. and 5% CO₂. Cells were passaged at 80% confluency using 0.25% trypsin-EDTA.

### Flow Cytometry

Hela cells were plated at 10,000 cells per well in a 96-well plate the night before the experiment. On the day of, cells were treated with the indicated concentrations of TAMRA-Omomyc, 7, 8, or 9 for 15 minutes in serum-containing culture medium, washed once with PBS, and treated with 0.25% trypsin-EDTA for 30 minutes to digest membrane-bound protein, at 37° C. and 5% CO₂. Cells were then washed with PBS, incubated in PBS containing 1× DAPI for three minutes, and then resuspended in PBS containing 2% FBS. Cells were then immediately analyzed on a BD FACS LSR II using DAPI and PE channels.

### Microscopy

Hela cells were plated at 10,000 cells/well in a 96-well 30 mm glass-bottom plate the night before the experiment. On the day of, cells were treated with TAMRA-Omomyc, 7, 8, or 9 (5 μM) in complete medium for 15 minutes, washed twice with fresh medium, and incubated at 37° C. and 5% CO₂ for 1 h before imaging. Micrographs were obtained in the W.M. Keck microscopy facility on an RPI Spinning Disk Confocal microscope on RFP setting (561 nm 100 mW OPSL excitation laser, 605/70 nm emission) and DAPI setting (405 nm 100 mW OPSL excitation laser, 450/50 nm emission).

### Example 4

### Inhibition of Cancer Cell Proliferation and MYC-Driven Transcription Upon Omomyc-Omomyc Treatment

The covalent protein dimers inhibit the proliferation of cancer cells. MYC is known to drive cell proliferation in the majority of human cancers. The bioactivity of all compounds was tested in three cell lines with a range of MYC expression levels (see Example 3 for cell culture protocols); HeLa contains high MYC levels, A549 contains mid-level, and H441 has low MYC expression. The cells were treated for 72 hours with covalent protein dimers and the proliferation was measured with a CellTiter-Glo® (CTG) assay. Cell proliferation inhibition followed the expected trend according to MYC expression levels; the most substantial inhibition was observed in Hela cells and the weakest in H441 cells. All synthetic dimers demonstrated inhibitory activity, with 4 having the highest activity with an EC₅₀ of 4 μM. This observation is in line with the structural stability data. Moreover, 9 further decreased the EC₅₀ in each cell line (2 μM in Hela cells), indicating that the nuclear-targeting moiety assists the transcription factor in reaching its target and imparts enhanced activity.

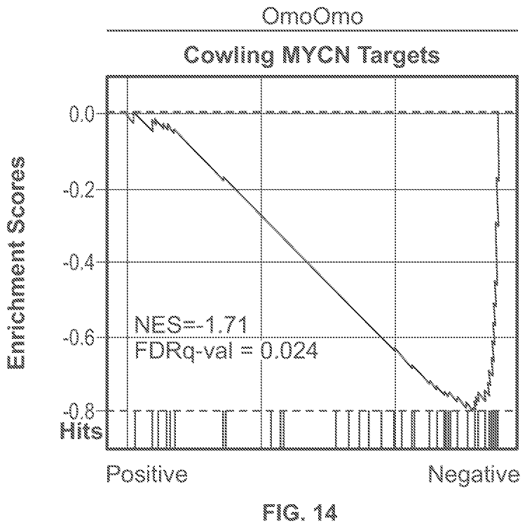

The covalent protein dimers interfere with MYC-driven gene expression, as determined by RNA-sequencing (RNA-seq) and gene set enrichment analysis (GSEA). To evaluate whether the compounds' bioactivity is related to the suppression of MYC-driven expression, RNA-seq was performed on A549 cells treated with 4. Compared to the control cells, downregulation of 431 and upregulation of 297 genes was observed, indicating that the covalent protein dimers have an effect on gene expression (FIG. 13). Among the downregulated genes, several genes involved in KRas signaling pathways were found, which are known to drive cancer development in A549 non-small lung cancer cells. This finding is in accordance with previous reports showing that MYC is a dominant effector of KRas mutation-positive lung cancer pathogenesis. GSEA of the RNA-seq data shows a negative enrichment of MYC-target gene set in the 4-treated condition, further corroborating that the covalent protein dimers interfere with MYC-driven gene expression programs (FIG. 14).

### Cell Proliferation Inhibition Assay

Cells were plated at 5,000 cells/well in a 96-well plate the day before the experiment. Covalent protein dimers were prepared at varying concentrations in complete media and transferred to the plate. Cells were incubated at 37° C. and 5% CO₂ for 72 h and cell proliferation was measured using the CellTiter-Glo assay quantified by luminescence.

### NA-Seq and GSEA

In a 6 well plate, 125,000 A549 cells were plated into each well. The following day, the cells were treated with 4 (12.5 μM) in F12K media supplemented with 10% FBS and 1% pen/strep and incubated for 72 h. RNA was isolated using the Qiagen RNeasy Plus Mini Kit (74136) followed by DNAse treatment (AM1906). KAPAHyperRiboErase libraries were prepared and sequenced on a Hi-seq 2500 instrument. Reads from sequencing were aligned using HISAT2 htseq-count function. Differential gene expression analysis between treated and control cells was performed using DESEQ2 package in R on raw aligned read counts. The differentially expressed genes were ranked by their log2FC and adjusted p-value. Pre-ranked Gene Set Enrichment Analysis (GSEA) was performed using gene sets in Molecular Signatures Database (MSigBD) to identify MYC-target gene sets.

### Example 5

### Synthesis of MAX, MYC, and Omomyc Analogs

### Preparation of MAX, MYC, and Omomyc Analogs 10, 11, and 12

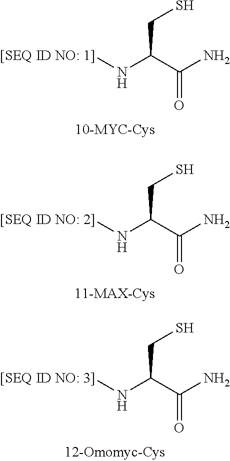

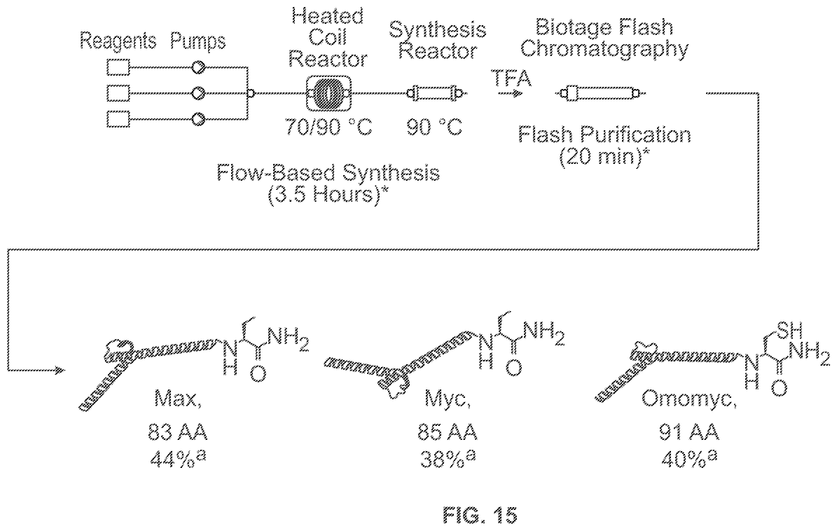

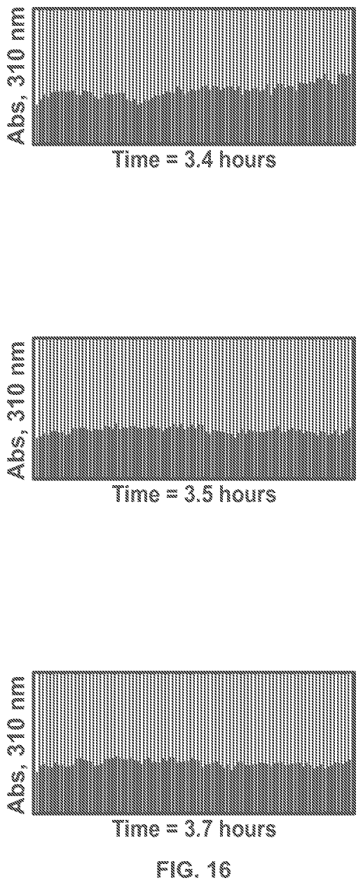

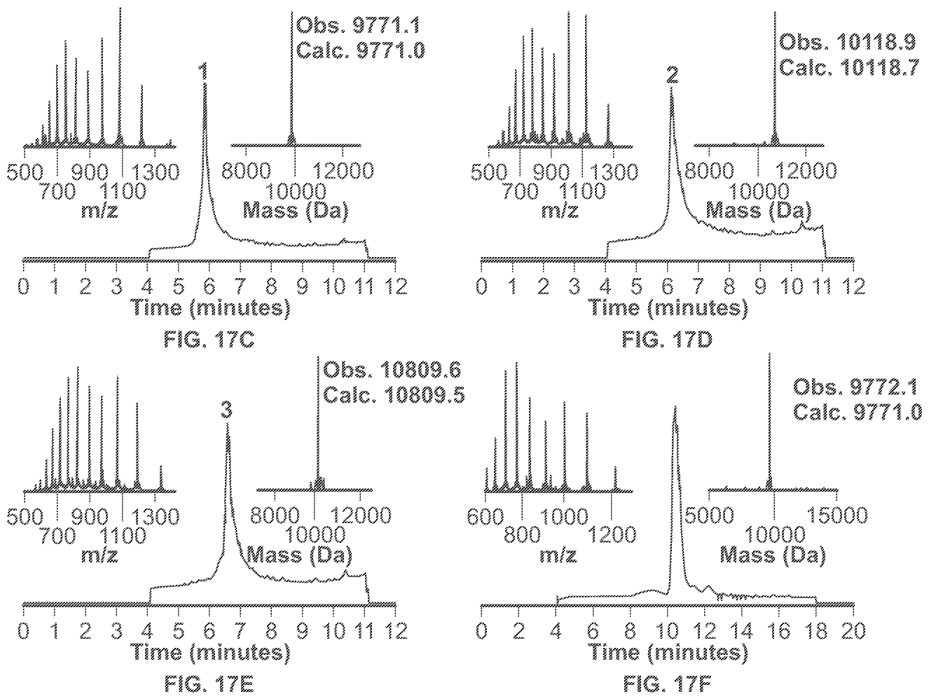

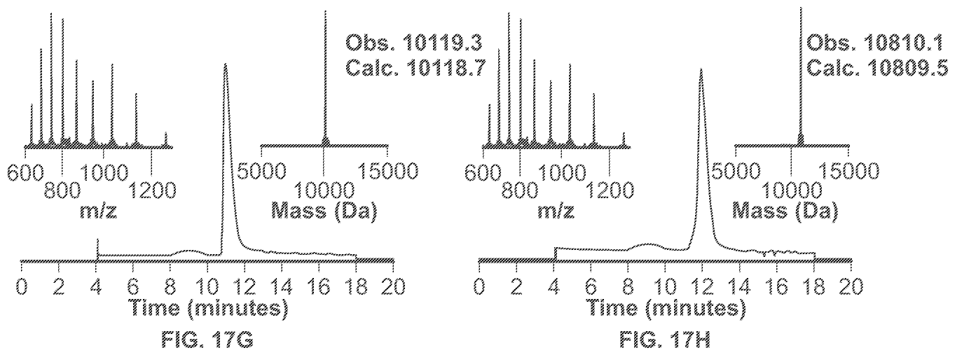

Stepwise automated fast-flow solid-phase synthesis (as described in Example 1) enabled rapid high-fidelity synthesis of Max (10), Myc (11), and Omomyc (12) analogs (83 to 91 residues in length; FIG. 15). The synthesis time for each protein amounted to ˜3.5 hours, and the three proteins were generated in one working day. A C-terminal cysteine residue was incorporated in the three analogs to allow subsequent cross-coupling reactions through palladium mediated S-arylation chemistry. In-line UV-vis detection of the Fmoc deprotection step after each coupling cycle indicated efficient incorporation of all monomers (FIG. 16). High quality syntheses of the three analogs was confirmed by LC-MS analysis of the crude products, after trifluoroacetic acid (TFA) cleavage and ether precipitation (FIG. 17). Analogs 10, 11, and 12 were purified via reversed-phase flash-chromatography, obtaining tens of milligrams of each of the three purified analogs in 38%, 44%, and 40% isolated yield, respectively. These results indicate that flow-based synthesis enables the generation of DNA-binding domains of Myc, Max, and Omomyc proteins.

### Example 6

### DNA-Binding Activity of MAX, MYC, and Omomyc Analogs

The three synthetic analogs 10, 11, and 12 can form non-covalent dimers and bind to the target E-box DNA (5′-CCGGCTGACACGTGGTATTAAT-3′). The DNA-binding activity of 10, 11, and 12 toward the canonical E-box sequence was determined by combining the analogs in all possible binary combinations (Max+Max, Myc+Myc, Omomyc+Omomyc, Myc+Max, Omomyc+Max, and Omomyc+Myc (FIG. 18). Each of the resulting six solutions was incubated individually with a 22 bp double-stranded DNA E-box sequence and the DNA-binding activity was examined by electrophoretic mobility shift assay (EMSA, FIG. 19). The synthetic proteins, with the exception of Myc 10, complexed with the E-Box DNA probe as indicated by a significant upward shift. As expected, Myc 10 alone does not bind to the E-box DNA probe because it cannot homodimerize. This DNA-binding assay suggests that each monomer is able to dimerize as expected and form functional protein complexes with E-box DNA. However, it is not possible to determine which dimeric species form in a solution containing two different monomers. For instance, upon mixing Max (11) and Omomyc (12), three different dimers (Max/Max, Omomyc/Omomyc and Max/Omomyc) can form and potentially bind to DNA (FIG. 18). To assess the activity of these protein complexes in a reliable manner, access to well-defined covalent protein dimers is critical.

### Example 7

### Synthesis and Characterization of Dithiobenzene-Linked Covalent Protein Dimers

### Cross-Coupling of TF Monomers Using Bifunctional Palladium OACs

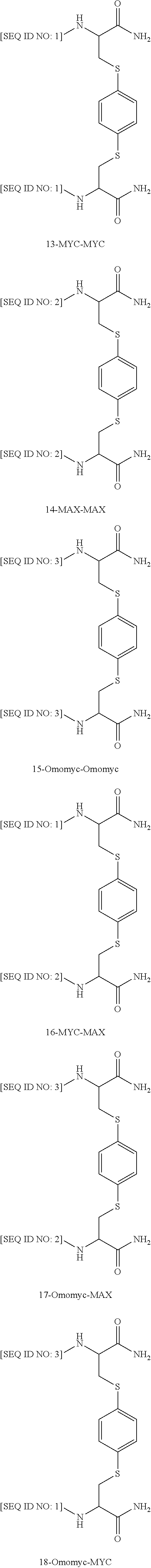

Bifunctional palladium oxidative addition complexes (OACs) enabled on-demand synthesis of homo- and heterodimeric analogs of the proteins 10, 11, and 12 to generate all possible covalent dimeric combinations. The dimerization strategy is shown in FIG. 20: the reaction of bifunctional Pd OAC with a protein monomer and subsequent palladium reinsertion into the aryl-iodide bond results in a protein-OAC that can then react with the cysteine of a second protein monomer, forming the final dimer. A single-flask protocol was used to form the homodimeric analogs. Each of the proteins 10, 11, and 12 was independently reacted with Pd OAC in 10% DMF, 20 mM Tris, 150 mM NaCl buffer (pH 7.5) at room temperature for 60 min (FIG. 21) to obtain the protein homodimers, as confirmed by SDS-PAGE. The homodimers were then purified via RP-HPLC and characterized by LC-MS analysis, affording Myc-Myc (13), Max-Max (14), and Omomyc-Omomyc (15) in 37%, 40%, and 38% isolated yield, respectively (FIG. 22).

To prepare the heterodimeric analogs, a two-step procedure was used by isolating the intermediate protein-OACs. The proteins 10, 11, and 12 were reacted with five equivalents of Pd OAC at room temperature for 60 min (FIG. 20) and the resulting protein-OAC intermediates were isolated via RP-HPLC. 10-OAC, 11-OAC, and 12-OAC were obtained in 45%, 54%, and 43% isolated yield, respectively. Next, each intermediate was reacted with the desired analog (10-OAC with 12, 11-OAC with 10, and 12-OAC with 11, FIG. 21). Finally, the heterodimer products were purified by RP-HPLC to provide the Myc-Max (16), Omomyc-Max (17), and Omomyc-Myc (18) analogs in 7%, 16%, and 6% isolated yield, respectively. The identity and purity of all six dimers were confirmed by LC-MS (FIG. 22) and SDS-PAGE analysis (FIG. 23). Next, the chemical stability of the S-aryl linkage was investigated. Protein dimer 14 (25 μM) was incubated in phosphate-buffered saline (PBS, pH 7.5) at 37° C. LC-MS analysis showed that no degradation had occurred after 24 h.

The covalent protein dimers exhibited α-helical character and displayed higher thermal stability compared to the monomeric analogs. The folding and stability of the dimeric analogs (13, 14, 15, 16, 17, and 18) was characterized via circular dichroism (CD) spectroscopy (see Example 2 for protocol). Strong double minima at 207 and 222 nm indicate an α-helical character of the dimeric analogs (FIG. 24). Analysis of the melting temperature (Tm) showed the dimers formed more thermodynamically stable complexes, indicated by the increase in Tm compared to the monomeric analogs (FIG. 25 and FIG. 26). Interestingly, the Omomyc-Max dimer 17 showed the highest Tm of 63° C., followed by Myc-Max 16 (53° C.) and Max-Max 14 (40° C.), compared to Max monomer 11, which was found to be 30° C. Omomyc-Omomyc 15, however, showed a similar Tm as the Omomyc monomer 12 at 59° C., likely due to the high propensity for homodimerization of the Omomyc protein. Overall, these results show that the S-aryl linkage can result in a structural stabilization of the dimeric protein complexes, compared to the monomeric analogs.

### General Strategy for Pd-Mediated Homodimer Synthesis

To a 1.5 mL Eppendorf tube was added Protein-Cys monomer (300 μL, 10.0 mg/mL, 1.0 equiv) as a solution in 20 mM Tris, 150 mM NaCl (pH 7.5), 234 μL 20 mM Tris, 150 mM NaCl (pH 7.5), 30.5 μL DMF and Pd OAC 4 (28.5 μL, 10.0 mg/mL, 1.0 equiv) as a solution in DMF (titrated over one minute). The final reaction concentrations of the major reaction components were the following: 2 (500 μM); 4 (500 μM). The Eppendorf tube was closed, vortexed, and incubated at room temperature for 60 min. A small aliquot was taken from the reaction mixture for analysis by SDS-PAGE. Finally, the reaction was quenched by DTT (10 μl, 1 M in H₂O) and kept at room temperature for 5 min, then purified by RP-HPLC.

### General Strategy for Protein-OAC Synthesis

To a Falcon 15 ml Conical Centrifuge Tube was added Protein monomer (9.0 mL, 1.1 mg/ml, 1.0 equiv) as a solution in 20 mM Tris, 150 mM NaCl (pH 7.5), and Pd OAC (1.0 mL, 4.8 mg/mL, 5.0 equiv) as a solution in DMF. The final reaction concentrations of the major reaction components were the following: 10 (100 μM); Pd OAc (500 μM). The Falcon Tube was closed vortexed and incubated at room temperature for 30 min. A small aliquot was taken from the reaction mixture for analysis by LC-MS. Finally, the reaction was purified by RP-HPLC.

### General Strategy for Pd-Mediated Heterodimer Synthesis

To a 5.0 mL Eppendorf tube was added protein-OAC (500 μL, 6.0 mg/mL, 1.0 equiv) as a solution in 20 mM Tris, 150 mM NaCl (pH 7.5), 260 μl 20 mM Tris, 150 mM NaCl buffer (pH 7.5), 185 μl DMF, and protein-Cys monomer (905 μl, 6.0 mg/ml, 2.0 equiv) as a solution in 20 mM Tris, 150 mM NaCl buffer (pH 7.5). The final reaction concentrations of the major reaction components were the following: protein-OAC (150 μM); protein-Cys (300 μM). The Eppendorf tube was closed, vortexed and incubated at room temperature for 60 min. A small aliquot was taken from the reaction mixture for analysis by SDS-PAGE. Finally, the reaction was quenched by DTT (10 μl, 1 M in H₂O) and kept at room temperature for 5 min, then purified by RP-HPLC.

### Example 8

### DNA-Binding Activity and Biophysical Characterization of the Protein Dimers

The S-aryl crosslinked protein dimers displayed DNA-binding activity to the E-box sequence. By EMSA, DNA association of Max-Max 14, Myc-Max 16, and Omomyc-Max 17 was observed (FIG. 27). No DNA binding was detected with negative control Myc-Myc 13. Also, Omomyc-Myc 18 showed no association to DNA, suggesting that this dimers' inhibitory activity might be related to its sequestering endogenous Myc into an inactive form. Finally, the dissociation constant of Max-Max 14 to the E-box DNA probe was measured by bio-layer interferometry (BLI) (FIG. 28). A K_{D} of 50±11 nM was determined, which is in good agreement with previous reports showing low nanomolar K_{D} values for E-box Max/Max complexes. Together these experiments demonstrate that the dimeric proteins form complexes with the E-box DNA with similar efficiency as the non-covalent analogs. Specifically, Max-Max 14 was identified as the closest analog to the natural Myc inhibitor Max/Max, as a potent binder for the E-box DNA.

### Example 9

### Cell-Permeability and Anti-Proliferative Activity of Covalent Protein Dimer 14

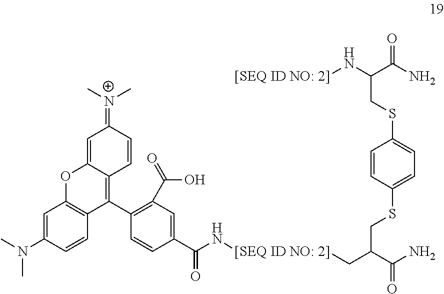

Cell-based studies revealed that the Max-Max 14 covalent protein dimer is intrinsically cell-permeable. To study Max-Max 14 bioactivity, the cross-coupling reaction was scaled up to generate ˜10 mg pure material for cellular studies. To assess the cell permeability, a Max-Max analog labeled with a single carboxytetramethylrhodamine (TAMRA) fluorophore (TAMRA-Max-Max (19) was prepared. Next, the cellular uptake of TAMRA-Max-Max 19 at varying concentrations was determined via flow-cytometry (see Example 3 for protocols). It was found that treatment with dimer 19 produced a dose-dependent increase in fluorescence, indicating that the dimer is taken up into cells (FIG. 29). Inclusion of DAPI as a viability stain did not result in a population of stained cells, indicating that the cells treated with TAMRA-Max-Max 19 did not suffer from membrane permeabilization. Further investigation of the uptake of the construct in Hela cells by confocal fluorescence microscopy demonstrated strong internalized fluorescence after a short incubation time (see Example 3 for protocols) (FIG. 30). These results establish that the synthetic dimer does not require further engineering to be directly delivered into the cell.

In addition to entering cells, Max-Max 14 also inhibits the proliferation of Myc-dependent cancer cell lines. In some cancer cell lines, such as HeLa, high levels of Myc drive robust cell proliferation. Covalent dimer 14 was tested in in Hela cells, which contain high levels of Myc, and cell proliferation was measured after 72 h. Covalent dimer 14 was found to inhibit HeLa cell proliferation in a dose-dependent manner with an EC₅₀ of 6 μM. The EC₅₀ of Max-Max 14 is in line with recent studies reporting small molecules for stabilizing endogenous Max dimer in cancer cell lines. Remarkably, in addition to its cell permeability, Max-Max 14 has comparable activity to small molecule-based inhibitors for Myc. Max-Max 14 was also found to inhibit the proliferation of lung adenocarcinoma cells A549 and H441 with EC₅₀ of 19 μM for both. Both of these lung cancer cell lines are known to have lower Myc levels compared to HeLa, which might explain the lower antiproliferative effect of Max-Max 14 in these cells assuming equivalent cell penetration. Taken together, these experiments suggest that Max-Max 14 enters the cells and inhibits cancer cell proliferation potentially by occupying the E-box site and blocking Myc-dependent gene transcription.

### Example 10

### RNA-Sequencing and Gene Set Enrichment Analysis for Cancer Cells Treated with Max-Max

RNA-sequencing analysis revealed Max-Max 14 selectively downregulates Myc-target genes in cancer cells. Myc is known to drive cell proliferation by triggering the expression of pro-proliferative genes through binding to the E-box DNA sequences. To assess the antiproliferative activity of Max-Max 14 through regulating Myc-driven genes, lung adenocarcinoma cells A549 were treated with Max-Max 14 for 72 h, and the RNA was extracted for RNA-sequencing analysis (see Example 4 for protocol). It was found that 14 directly interferes with gene transcription by downregulating 160 genes and upregulating 70 genes (FIG. 32). The identified down- and up-regulated genes are in agreement with previous reports of Myc inhibition. Remarkably, Max-Max 14 was found to downregulate the expression of several genes involved in KRas signaling pathways that often progresses cancer. The selectivity of 14 toward Myc-related genes was further confirmed by gene set enrichment analysis (GSEA) of the RNA-sequencing data with several Myc target gene sets. Taken together, these results confirm that synthetic complex 14 is capable of downregulating Myc-driven gene signatures.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

### REFERENCES

1. Dawson, P. E., Muir, T. W., Clark-Lewis, I. & Kent, S. B. H. Synthesis of proteins by native chemical ligation. Science (80-.). 266, 776 (1994).

2. Bode, J. W., Fox, R. M. & Baucom, K. D. Chemoselective amide ligations by decarboxylative condensations of N-alkylhydroxylamines and α-ketoacids. Angew. Chemie-Int. Ed. 45, 1248-1252 (2006).

3. Premdjee, B., Andersen, A. S., Larance, M., Conde-Frieboes, K. W. & Payne, R. J. Chemical Synthesis of Phosphorylated Insulin-like Growth Factor Binding Protein 2. J. Am. Chem. Soc. 143, 5336-5342 (2021).

4. Agouridas, V. et al. Native Chemical Ligation and Extended Methods: Mechanisms, Catalysis, Scope, and Limitations. Chem. Rev. 119, (2019).

5. Conibear, A. C., Watson, E. E., Payne, R. J. & Becker, C. F. W. Native chemical ligation in protein synthesis and semi-synthesis. Chem. Soc. Rev. 47, 9046-9068 (2018).

6. Bondalapati, S., Jbara, M. & Brik, A. Expanding the chemical toolbox for the synthesis of large and uniquely modified proteins. Nat. Chem. 8, 407-418 (2016).

7. Bertolini, M. et al. Interactions between nascent proteins translated by adjacent ribosomes drive homomer assembly. Science (80-.). 371, 57-64 (2021).

8. Shiber, A. et al. Cotranslational assembly of protein complexes in eukaryotes revealed by ribosome profiling. Nature 561, 268-272 (2018).

9. Meyer, N. & Penn, L. Z. Reflecting on 25 years with MYC. Nat. Rev. Cancer 8, 976-990 (2008).

10. Blackwell, T., Kretzner, L., Eisenman, R., Weintraub, H. & Blackwood, E. Sequence-specific DNA binding by the c-Myc protein. Science (80-.). 250, 1149-1151 (2006).

11. Blackwood, E. M. & Eisenman, R. N. Max: A Helix-Loop-Helix Zipper Protein That Complex with Myc. Science (80-.). 251, 1211-1217 (1991).

12. Ferré-D′Amaré, A. R., Prendergast, G. C., Ziff, E. B. & Burley, S. K. Recognition by Max of its cognate DNA through a dimeric b/HLH/Z domain. Nature 363, 38-45 (1993).

13. Chen, H., Liu, H. & Qing, G. Targeting oncogenic Myc as a strategy for cancer treatment. Signal Transduct. Target. Ther. 3, 1-7 (2018).

14. Kalkat, M. et al. MYC deregulation in primary human cancers. Genes (Basel). 8, 2-30 (2017).

15. Rahl, P. B. et al. Transcriptional Amplification in Tumor Cells with Elevated c-Myc. (2012). doi: 10.1016/j.cell.2012.08.026

16. Lee, T. I. & Young, R. A. Transcriptional regulation and its misregulation in disease. Cell 152, 1237-1251 (2013).

17. Fletcher, S. & Prochownik, E. V. Small-molecule inhibitors of the Myc oncoprotein. Biochim. Biophys. Acta—Gene Regul. Mech. 1849, 525-543 (2015).

18. Boike, L. et al. Discovery of a Functional Covalent Ligand Targeting an Intrinsically Disordered Cysteine within MYC. Cell Chem. Biol. 1-10 (2020). doi: 10.1016/j.chembiol.2020.09.001

19. Han, H. et al. Small-Molecule MYC Inhibitors Suppress Tumor Growth and Enhance Immunotherapy. Cancer Cell 36, 483-497.e15 (2019).

20. Koehler, A. N. A complex task? Direct modulation of transcription factors with small molecules. Curr. Opin. Chem. Biol. 14, 331-340 (2010).

21. Ulasov, A. V., Rosenkranz, A. A. & Sobolev, A. S. Transcription factors: Time to deliver. J. Control. Release 269, 24-35 (2018).

22. Madden, S. K., de Araujo, A. D., Gerhardt, M., Fairlie, D. P. & Mason, J. M. Taking the Myc out of cancer: toward therapeutic strategies to directly inhibit c-Myc. Mol. Cancer 20, 1-18 (2021).

23. Struntz, N. B. et al. Stabilization of the Max Homodimer with a Small Molecule Attenuates Myc-Driven Transcription. Cell Chem. Biol. 26, 711-723.e14 (2019).

24. Soucek, L. et al. Design and properties of a Myc derivative that efficiently homodimerizes. Oncogene 17, 2463-2472 (1998).

25. Massó-Vallés, D. & Soucek, L. Blocking Myc to Treat Cancer: Reflecting on Two Decades of Omomyc. Cells 9, 883 (2020).

26. Beaulieu, M. E. et al. Intrinsic cell-penetrating activity propels omomyc from proof of concept to viable anti-myc therapy. Sci. Transl. Med. 11, 1-14 (2019).

27. Demma, M. J. et al. Omomyc Reveals New Mechanisms To Inhibit the MYC Oncogene. Mol. Cell. Biol. 39, 1-27 (2019).

28. Lobba, M. J. et al. Site-Specific Bioconjugation through Enzyme-Catalyzed Tyrosine—Cysteine Bond Formation. ACS Cent. Sci. (2020). doi: 10.1021/acscentsci.0c00940

29. Dhanjee, H. H. et al. Protein-Protein Cross-Coupling via Palladium-Protein Oxidative Addition Complexes from Cysteine Residues. J. Am. Chem. Soc. 142, 9124-9129 (2020).

30. Kumar, K. S. A., Spasser, L., Erlich, L. A., Bavikar, S. N. & Brik, A. Total chemical synthesis of di-ubiquitin chains. Angew. Chemie—Int. Ed. 49, 9126-9131 (2010).

31. Chatterjee, C., McGinty, R. K., Pellois, J.-P. & Muir, T. W. Auxiliary-Mediated Site-Specific Peptide Ubiquitylation. Angew. Chemie 119, 2872-2876 (2007).

32. Ajish Kumar, K. S., Haj-Yahya, M., Olschewski, D., Lashuel, H. A. & Brik, A. 32. Highly efficient and chemoselective peptide ubiquitylation. Angew. Chemie—Int. Ed. 48, 8090-8094 (2009).

33. Fottner, M. et al. Site-specific ubiquitylation and SUMOylation using genetic-code expansion and sortase. Nat. Chem. Biol. 15, 276-284 (2019).

34. Sui, X. et al. Development and application of ubiquitin-based chemical probes. Chem. Sci. 11, 12633-12646 (2020).

35. Geurink, P. P., El Oualid, F., Jonker, A., Hameed, D. S. & Ovaa, H. A General Chemical Ligation Approach Towards Isopeptide-Linked Ubiquitin and Ubiquitin-Like Assay Reagents. ChemBioChem 13, 293-297 (2012).

36. Kulkarni, S. S., Sayers, J., Premdjee, B. & Payne, R. J. Rapid and efficient protein synthesis through expansion of the native chemical ligation concept. Nat. Rev. Chem. 2, 1-17 (2020).

37. Pan, M. et al. Quasi-Racemic X-ray Structures of K27-Linked Ubiquitin Chains Prepared by Total Chemical Synthesis. J. Am. Chem. Soc. 138, 7429-7435 (2016).

38. Torbeev, V. Y. et al. Protein conformational dynamics in the mechanism of HIV-1 protease catalysis. Proc. Natl. Acad. Sci. U. S. A. 108, 20982-20987 (2011).

39. Nair, S. K. & Burley, S. K. X-ray structures of Myc-Max and Mad-Max recognizing DNA: Molecular bases of regulation by proto-oncogenic transcription factors. Cell 112, 193-205 (2003).

40. Canne, L. E., Ferré-D′Amaré, A. R., Burley, S. K. & Kent, S. B. H. Total Chemical Synthesis of a Unique Transcription Factor-Related Protein: cMyc—Max. J. Am. Chem. Soc. 117, 2998-3007 (1995).

41. Mijalis, A. J. et al. A fully automated flow-based approach for accelerated peptide synthesis. Nat. Chem. Biol. 13, 464-466 (2017).

42. Hartrampf, N. et al. Synthesis of proteins by automated flow chemistry. Science 368, 980-987 (2020).

43. Palmacci, E. R., Plante, O. J., Hewitt, M. C. & Seeberger, P. H. Automated Synthesis of Oligosaccharides. Science (80-.). 291, 1523 (2001).

44. Schissel, C. et al. Interpretable Deep Learning for De Novo Design of Cell-Penetrating Abiotic Polymers. (2020). doi: 10.1101/2020.04.10.036566

45. Fadzen, C. M. et al. Chimeras of Cell-Penetrating Peptides Demonstrate Synergistic Improvement in Antisense Efficacy. Biochemistry 58, 3980-3989 (2019).

46. Wang, E. et al. Tumor penetrating peptides inhibiting MYC as a potent targeted therapeutic strategy for triple-negative breast cancers. Oncogene 38, 140-150 (2019).

47. Fukazawa, T. et al. Inhibition of myc effectively targets KRAS mutation-positive lung cancer expressing high levels of Myc. Anticancer Res. 30, 4193-4200 (2010).

48. Spiegel, J., Cromm, P. M., Zimmermann, G., Grossmann, T. N. & Waldmann, H. Small-molecule modulation of Ras signaling. Nat. Chem. Biol. 10, 613-622 (2014).

49. Johnson, C. D. et al. The let-7 microRNA represses cell proliferation pathways in human cells. Cancer Res. 67, 7713-7722 (2007).

50. Blackwood, E. M. & Eisenman, R. N. Max: A helix-loop-helix zipper protein that forms a sequence-specific DNA-binding complex with Myc. Science (80-.). 251, 1211-1217 (1991).

51. Amati, B. et al. Transcriptional activation by the human c-Myc oncoprotein in yeast requires interaction with Max. Nature 359, 423-426 (1992).

52. Hu, J., Banerjee, A. & Goss, D. J. Assembly of b/HLH/z proteins c-Myc, Max, and Mad1 with cognate DNA: Importance of protein-protein and protein-DNA interactions. Biochemistry 44, 11855-11863 (2005).

53. Montagne, M. et al. The max b-HLH-LZ can transduce into cells and inhibit c-Myc transcriptional activities. PLOS One 7, 2-10 (2012).

54. Demma, M. J. et al. Inhibition of Myc transcriptional activity by a mini-protein based upon Mxd1. FEBS Lett. 594, 1467-1476 (2020).

1. A covalent protein dimer, or a pharmaceutically acceptable salt thereof, comprising:
a first polypeptide comprising a C-terminus and an N-terminus, wherein the first polypeptide comprises a degree of identity of at least 85% with respect to SEQ ID NO: 1, 2, or 3;
a second polypeptide comprising a C-terminus and an N-terminus, wherein the second polypeptide comprises a degree of identity of at least 85% with respect to SEQ ID NO: 1, 2, or 3; and
a linker covalently linking the C-terminus of the first polypeptide to the C-terminus of the second polypeptide.

2. The covalent protein dimer of claim 1, wherein:
the first polypeptide is at least 85% identical to SEQ ID NO: 2, and the second polypeptide is at least 85% identical to SEQ ID NO: 2;
the first polypeptide is at least 85% identical to SEQ ID NO: 3, and the second polypeptide is at least 85% identical to SEQ ID NO: 3;
the first polypeptide is at least 85% identical to SEQ ID NO: 1, and the second polypeptide is at least 85% identical to SEQ ID NO: 2; or
the first polypeptide is at least 85% identical to SEQ ID NO: 3, and the second polypeptide is at least 85% identical to SEQ ID NO: 2.

3. A covalent protein dimer, or a pharmaceutically acceptable salt thereof, having a structure according to Formula (I): 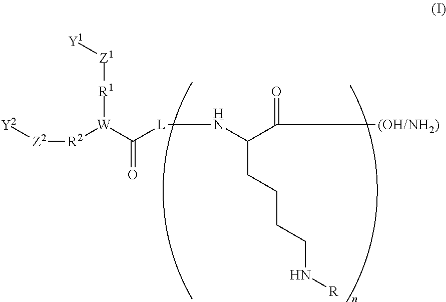
wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ is —O—, —NH—, or —S—;
Z² is —O—, —NH—, or —S—;
R¹ is absent, C₁₋₁₀ alkyl, or C₁₋₁₀ heteroalkyl;
R² is absent, C₁₋₁₀ alkyl, or C₁₋₁₀ heteroalkyl;
W is C₁₋₁₀ alkyl, C₁₋₁₀ heteroalkyl, C₆₋₁₀ aryl, or 5-to 10-membered heteroaryl;
L is absent or a linker;
R is H, a nitrogen protecting group, biotin, a fluorescent dye, a nuclear-targeting moiety, or a cell-penetrating moiety; and
n is 0 or 1.

4. The covalent protein dimer of claim 3 having a structure according to Formula (Ib): 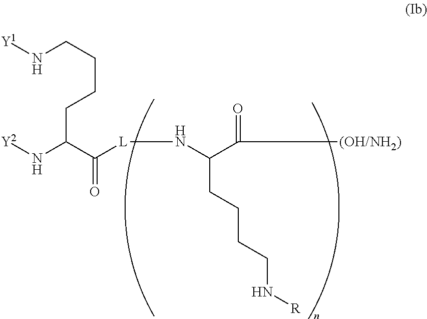
wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety; and
n is 0 or 1.

5. The covalent protein dimer of claim 4, wherein if one of Y¹ or Y² is at least 85% identical to SEQ ID NO: 1, then the other is at least 85% identical to SEQ ID NO: 2.

6. The covalent protein dimer of claim 4, wherein:
Y¹ is at least 85% identical to SEQ ID NO: 2, and Y² is at least 85% identical to SEQ ID NO: 2;
Y¹ is at least 85% identical to SEQ ID NO: 3, and Y² is at least 85% identical to SEQ ID NO: 3;
Y¹ is at least 85% identical to SEQ ID NO: 1, and Y² is at least 85% identical to SEQ ID NO: 2; or
Y¹ is at least 85% identical to SEQ ID NO: 3, and Y² is at least 85% identical to SEQ ID NO: 2.

7. The covalent protein dimer of claim 4, wherein L is a linker comprising one to fifty amino acids.

8. The covalent protein dimer of claim 7, wherein L is β-alanine.

9. The covalent protein dimer of claim 4, wherein R is a nitrogen protecting group that is not Fmoc.

10. The covalent protein dimer of claim 4, wherein R is a nitrogen protecting group comprising Alloc or Boc.

11. The covalent protein dimer of claim 4, wherein R is a fluorescent dye comprising 5-TAMRA.

12. The covalent protein dimer of claim 4, wherein R is a nuclear-targeting moiety comprising Mach3 comprising SEQ ID NO:
QKKRKSKANKKNWPKGKLSIHAKDYKQGPKAKXₐₐRKQRXₐₐRG (SEQ ID NO: 4), wherein Xₐₐ is 6-aminohexanoic acid.

13. A pharmaceutical composition comprising the covalent protein dimer of claim 4 and a pharmaceutically acceptable carrier.

14. A method of treating a disease or disorder characterized by MYC dysregulation in a subject in need thereof, the method comprising administering to the subject the covalent protein dimer of claim 4.

15. The method of claim 14, wherein the disease or disorder is cancer.

16. A method of making a covalent protein dimer, or a pharmaceutically acceptable salt thereof, having a structure according to Formula (Ib): 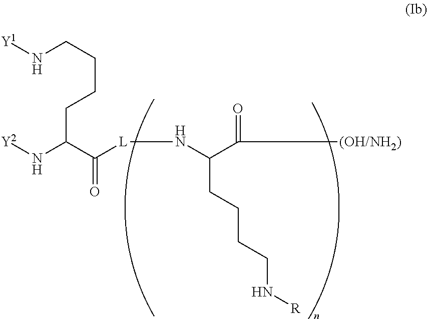
wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety;
n is 0 or 1;
the method comprising:
(a) reacting a first resin-bound, side-chain-protected peptide having a structure according to Formula (IIIb): 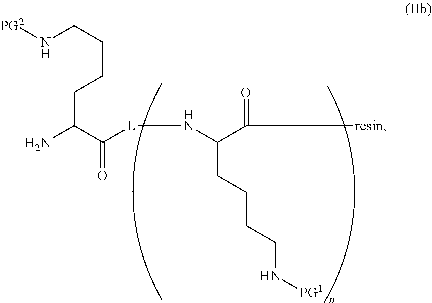
with one or more amino acids corresponding to the amino acids of the polypeptide represented by Y² to provide a second resin-bound, side-chain-protected peptide having a structure according to Formula (IVb): 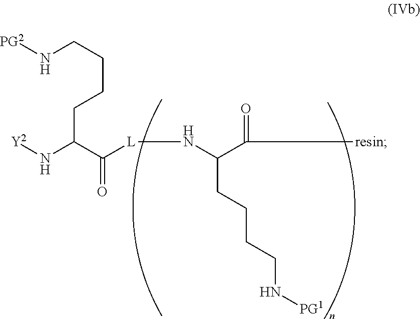
wherein PG¹ and PG² are non-identical nitrogen protecting groups, and wherein neither PG¹ nor PG² are Fmoc;
(b) removing PG² from the second resin-bound, side-chain-protected peptide to provide a third resin-bound, side-chain-protected peptide having a structure according to Formula (Vb): 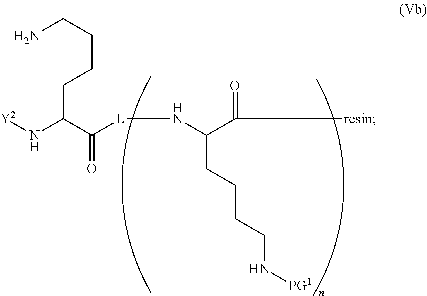
(c) reacting the third resin-bound, side-chain-protected peptide with one or more amino acids corresponding to the amino acids of the polypeptide represented by Y¹ to provide a fourth resin-bound, side-chain protected peptide having a structure according to Formula (VIb): 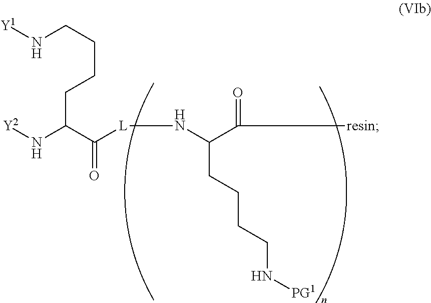
and
(d) cleaving the fourth resin-bound, side-chain-protected peptide from the resin to provide the covalent protein dimer.

17. The method of claim 16, wherein prior to step (d), the method comprises removing PG¹ to provide a deprotected nitrogen atom therein, and covalently attaching biotin, a fluorescent dye, a nuclear-targeting moiety, or a cell-penetrating moiety to the deprotected nitrogen atom.

18. The method of claim 16, wherein each of the one or more amino acids of steps (a) and (c) comprises an Fmoc-protected backbone amino group, and wherein the corresponding Fmoc group is deprotected after each amino acid is attached to the resin-bound, side-chain-protected peptide.

19. The method of claim 16, wherein each one of steps (a) and (c) is performed in the presence of a coupling agent.

20. The method of claim 19, wherein the coupling agent is selected from the group consisting of (7-azabenzotriazol-1-yloxy) tripyrrolidinophosphonium hexafluorophosphate (PyAOP), hexafluorophosphate azabenzotriazole tetramethyl uronium (HATU), hexafluorophosphate benzotriazole tetramethyl uronium (HBTU), 2-(6-chloro-1H-benzotriazole-1-yl)-1,1,3,3-tetramethylaminium hexafluorophosphate (HCTU), and hydroxybenzotriazole (HOBt).

21. The method of claim 16, wherein each one of steps (a) and (c) comprises the addition of N,N-Diisopropylethylamine (DIEA).

22. The method of claim 16, wherein PG¹ is Boc and PG² is Alloc.

23. A method of making a covalent protein dimer, or a pharmaceutically acceptable salt thereof, having a structure according to Formula (Ib): 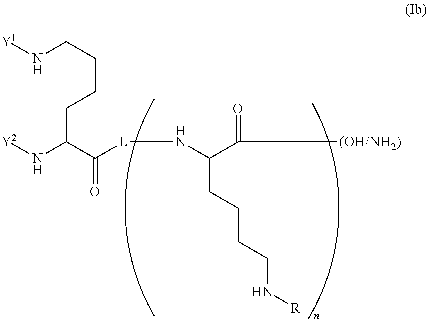
wherein:
Y¹ and Y² are identical and each represents a polypeptide comprising a degree of identity of at least 85% with respect to SEQ ID NO: 1, 2, or 3;
L is absent or a linker;
R is H, a nitrogen protecting group, biotin, a fluorescent dye, a nuclear-targeting moiety, or a cell-penetrating moiety;
n is 0 or 1;
the method comprising:
(a) reacting a first resin-bound, side-chain-protected peptide having a structure according to Formula (VIIb): 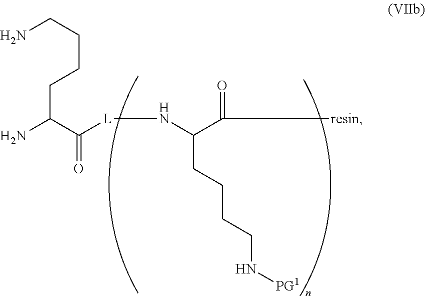
with one or more amino acids corresponding to the amino acids of the polypeptide represented by Y¹ to provide a second resin-bound, side-chain-protected peptide having a structure according to Formula (VIb): 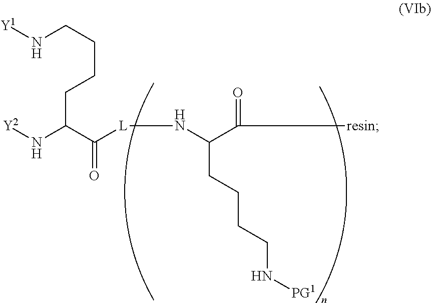
wherein PG¹ is a nitrogen protecting group that is not Fmoc; and
(b) cleaving the second resin-bound, side-chain-protected peptide from the resin to provide the covalent protein dimer.

24. The method of claim 23, wherein prior to step (b), the method comprises removing PG¹ to provide a deprotected nitrogen atom therein, and covalently attaching biotin, a fluorescent dye, a nuclear-targeting moiety, or a cell-penetrating moiety to the deprotected nitrogen atom.

25. The method of claim 23, wherein each of the one or more amino acids of step (a) comprise an Fmoc-protected backbone amino group, and wherein the corresponding Fmoc group is deprotected after each amino acid is attached to the resin-bound, side-chain-protected peptide.

26. The method of claim 23, wherein step (a) is performed in the presence of a coupling agent.

27. The method of claim 26, wherein the coupling agent is selected from the group consisting of (7-azabenzotriazol-1-yloxy) tripyrrolidinophosphonium hexafluorophosphate (PyAOP), hexafluorophosphate azabenzotriazole tetramethyl uronium (HATU), hexafluorophosphate benzotriazole tetramethyl uronium (HBTU), 2-(6-chloro-1H-benzotriazole-1-yl)-1,1,3,3-tetramethylaminium hexafluorophosphate (HCTU), and hydroxybenzotriazole (HOBt).

28. The method of claim 23, wherein step (a) comprises the addition of N,N-Diisopropylethylamine (DIEA).

29. The method of claim 23, wherein PG¹ is Alloc.

30. A covalent protein dimer, or a pharmaceutically acceptable salt thereof, having a structure according to Formula (II): 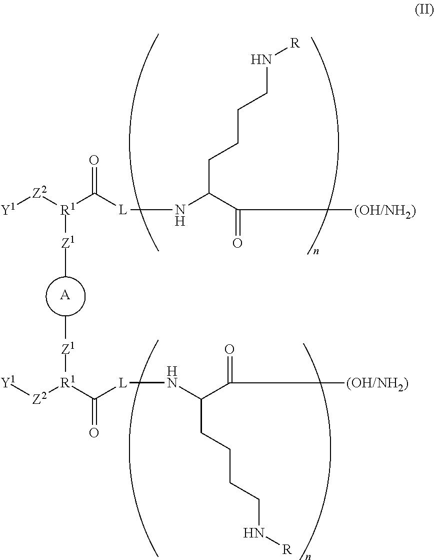
wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
Z¹ independently is —O—, —NH—, or —S—;
Z² independently is —O—, —NH—, or —S—;
R¹ independently is C₁₋₁₀ alkyl or C₁₋₁₀ heteroalkyl;
A is C₈₋₁₀ aryl or 5- to 10-membered heteroaryl;
L independently is absent or a linker;
R independently is H, a nitrogen protecting group, biotin, a fluorescent dye, a nuclear-targeting moiety, or a cell-penetrating moiety; and
n independently is 0 or 1.

31. The covalent protein dimer of claim 30 having a structure according to Formula (IIa): 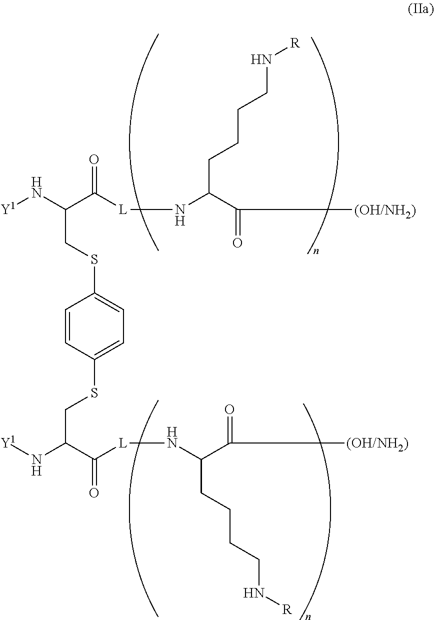
wherein:
Y¹ is a polypeptide comprising a degree of identity of at least 85% with respect to SEQ ID NO: 1, 2, or 3;
Y² is a polypeptide comprising a degree of identity of at least 85% with respect to SEQ ID NO: 1, 2, or 3;
L independently is absent or a linker;
R independently is H, a nitrogen protecting group, biotin, a fluorescent dye, a nuclear-targeting moiety, or a cell-penetrating moiety; and
n independently is 0 or 1.

32. The covalent protein dimer of claim 31 having a structure according to Formula (IIb): 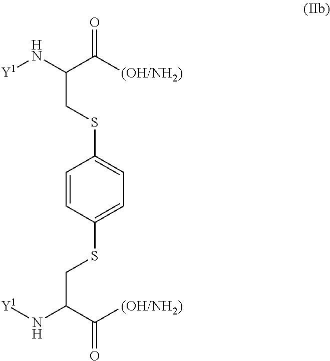
wherein:
Y¹ is a polypeptide comprising a degree of identity of at least 85% with respect to SEQ ID NO: 1, 2, or 3; and
Y² is a polypeptide comprising a degree of identity of at least 85% with respect to SEQ ID NO: 1, 2, or 3.

33. The covalent protein dimer of claim 32, wherein:
Y¹ is at least 85% identical to SEQ ID NO: 2, and Y² is at least 85% identical to SEQ ID NO: 2;
Y¹ is at least 85% identical to SEQ ID NO: 3, and Y² is at least 85% identical to SEQ ID NO: 3;
Y¹ is at least 85% identical to SEQ ID NO: 1, and Y² is at least 85% identical to SEQ ID NO: 2; or
Y¹ is at least 85% identical to SEQ ID NO: 3, and Y² is at least 85% identical to SEQ ID NO: 2.

34. A pharmaceutical composition comprising the covalent protein dimer of claim 32 and a pharmaceutically acceptable carrier.

35. A method of treating a disease or disorder characterized by MYC dysregulation in a subject in need thereof, the method comprising administering to the subject the covalent protein dimer of claim 32.

36. The method of claim 35, wherein the disease or disorder is cancer.

37. A method of making a covalent protein dimer, or a pharmaceutically acceptable salt thereof, having a structure according to Formula (VIIb): 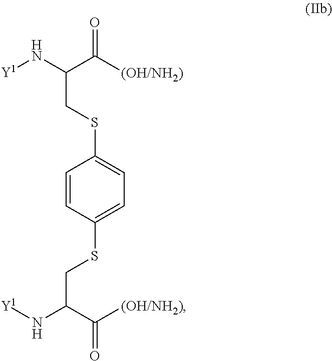
wherein:
Y¹ is a polypeptide comprising a degree of identity of at least 85% with respect to SEQ ID NO: 1, 2, or 3; and
Y² is a polypeptide comprising a degree of identity of at least 85% with respect to SEQ ID NO: 1, 2, or 3;
the method comprising:
(a) reacting a polypeptide having a structure according to Formula (VIIIb): 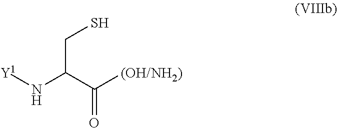
with a compound of Formula (IX): 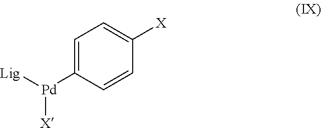
to provide a polypeptide having a structure according to Formula (Xb): 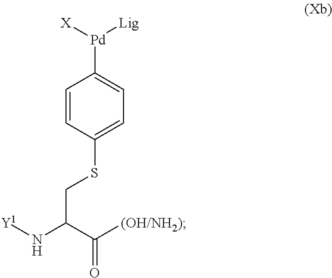
wherein:
X and X′ are each, independently, F, Cl, Br, I, or OTf; and
Lig is a phosphine ligand; and
(b) reacting the polypeptide of Formula (X) with a polypeptide having a structure according to Formula (XI): 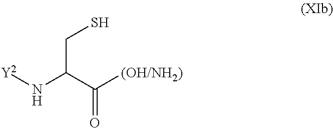
to provide the covalent protein dimer.

38. The method of claim 37, wherein the compound of Formula (IX) is provided in molar excess with respect to the polypeptide of Formula (VIII).

39. The method of claim 37, wherein X and X′ are I.

40. The method of claim 37, wherein Lig has a structure according to Formula (XII): 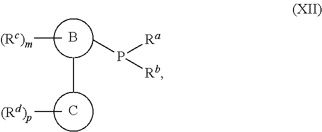
wherein:
B and C are each, independently, C₆₋₁₀ aryl or 6- to 10-membered heteroaryl;
R^{a} and R^{b} are each, independently, C₅₋₁₀ cycloalkyl, C₁₋₆ alkyl, or C₈₋₁₀ aryl, optionally wherein the aryl is substituted with one, two, or three C₁₋₃ haloalkyl groups;
R^{c}, independently is C₁₋₄ alkyl, C₁₋₄ alkoxy, or N (C₁₋₄ alkyl)₂
R^{d}, independently is C₁₋₄ alkyl, C₁₋₄ alkoxy, N(C₁₋₄ alkyl)₂, SO₃H, SO₃M, or C₃₋₁₀ cycloalkyl;
M is Li, Na, or K;
m is 0, 1, 2, 3, or 4; and
p is 1, 2, 3, or 4.

41. The method of claim 40, wherein Lig is:

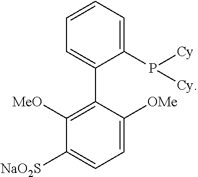

42. A method of making a covalent protein dimer, or a pharmaceutically acceptable salt thereof, having a structure according to Formula (IIb): 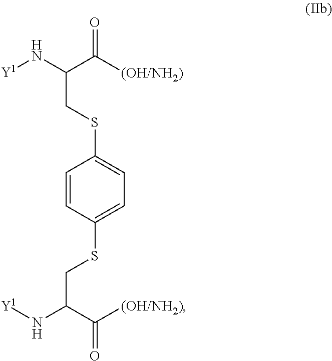
wherein:
Y¹ and Y² are identical and each represents a polypeptide comprising a degree of identity of at least 85% with respect to SEQ ID NO: 1, 2, or 3;
the method comprising reacting a polypeptide having a structure according to Formula (VIII): 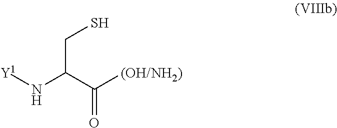
with a compound of Formula (IX): 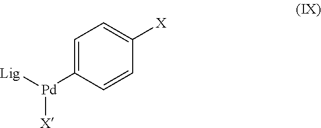
to provide the covalent protein dimer;
wherein:
X and X′ are each, independently, F, Cl, Br, I, or OTf; and
Lig is a phosphine ligand.

43. The method of claim 42, wherein X and X′ are I.

44. The method of claim 42, wherein Lig has a structure according to Formula (XII): 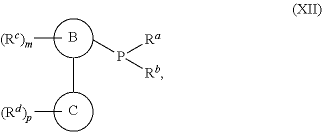
wherein:
B and C are each, independently, C₈₋₁₀ aryl or 6- to 10-membered heteroaryl;
R^{a} and R^{b} are each, independently, C₅₋₁₀ cycloalkyl, C₁₋₆ alkyl, or C₈₋₁₀ aryl, optionally wherein the aryl is substituted with one, two, or three C₁₋₃ haloalkyl groups;
R^{c}, independently is C₁₋₄ alkyl, C₁₋₄ alkoxy, or N(C₁₋₄ alkyl)₂
R^{d}, independently is C₁₋₄ alkyl, C₁₋₄ alkoxy, N(C₁₋₄ alkyl)₂, SO₃H, SO₃M, or C₃₋₁₀ cycloalkyl;
M is Li, Na, or K;
m is 0, 1, 2, 3, or 4; and
p is 1, 2, 3, or 4.

45. The method of claim 44, wherein Lig is: [chemical structure]
